SEQUENCE LISTING (186 sequences)

SEQ ID NO: 1 - protein, 7 aa, Artificial Sequence
  VHL-tag
  MISC_FEATURE (4)..(4): X is hydroxyproline
ALAXYIP

SEQ ID NO: 2 - protein, 12 aa, Artificial Sequence
  IkBa peptide
DRHDSPGLDSPM

SEQ ID NO: 3 - protein, 14 aa, Artificial Sequence
  Ligand
KFERQKILDQRFFE

SEQ ID NO: 4 - protein, 12 aa, Artificial Sequence
  Ligand
VKKDQGSKFERQ

SEQ ID NO: 5 - protein, 5 aa, Artificial Sequence
  Ligand
KFERQ

SEQ ID NO: 6 - protein, 8 aa, Artificial Sequence
  Ligand
NLLRLTGW

SEQ ID NO: 7 - protein, 6 aa, Artificial Sequence
  APR region tau protein
VQIVYK

SEQ ID NO: 8 - protein, 6 aa, Artificial Sequence
  APR region of tau protein
VQIINK

SEQ ID NO: 9 - protein, 6 aa, Artificial Sequence
  Capping peptide
WQIVYK

SEQ ID NO: 10 - protein, 11 aa, Artificial Sequence
  VHL-moiety
  MISC_FEATURE (8)..(8): X is hydroxyproline
GSGSALAXYIP

SEQ ID NO: 11 - protein, 12 aa, Artificial Sequence
  Javelin moiety
GSGSNLLRLTGW

SEQ ID NO: 12 - protein, 14 aa, Artificial Sequence
  Capping peptide
WQIVYKGSWQIVYK

SEQ ID NO: 13 - protein, 18 aa, Artificial Sequence
  Capping peptide
RWQIVYKRGSRWQIVYKR

SEQ ID NO: 14 - protein, 18 aa, Artificial Sequence
  Capping peptide
DWQIVYKDGSDWQIVYKD

SEQ ID NO: 15 - protein, 6 aa, Artificial Sequence
  Capping peptide
VQIVYP

SEQ ID NO: 16 - protein, 14 aa, Artificial Sequence
  Capping peptide
VQIVYPGSVQIVYP

SEQ ID NO: 17 - protein, 18 aa, Artificial Sequence
  Capping peptide
RVQIVYPRGSRVQIVYPR

SEQ ID NO: 18 - protein, 18 aa, Artificial Sequence
  Capping peptide
DVQIVYPDGSDVQIVYPD

SEQ ID NO: 19 - protein, 14 aa, Artificial Sequence
  Capping peptide
WQIVYKGSVWMINK

SEQ ID NO: 20 - protein, 18 aa, Artificial Sequence
  Capping peptide
RWQIVYKRGSRVWMINKR

SEQ ID NO: 21 - protein, 18 aa, Artificial Sequence
  Capping peptide
DWQIVYKDGSDVWMINKD

SEQ ID NO: 22 - protein, 26 aa, Artificial Sequence
  HET_JAV
WQIVYKGSVWMINKGSGSNLLRLTGW

SEQ ID NO: 23 - protein, 32 aa, Artificial Sequence
  HET_CMA1
WQIVYKGSVWMINKGSGSKFERQKILDQRFFE

SEQ ID NO: 24 - protein, 30 aa, Artificial Sequence
  HET_CMA2
WQIVYKGSVWMINKGSGSVKKDQGSKFERQ

SEQ ID NO: 25 - protein, 6 aa, Artificial Sequence
  Capping peptide
EQIINE

SEQ ID NO: 26 - protein, 6 aa, Artificial Sequence
  Capping peptide
VQWIIK

SEQ ID NO: 27 - protein, 6 aa, Artificial Sequence
  capping peptide
EDIINK

SEQ ID NO: 28 - protein, 6 aa, Artificial Sequence
  Capping peptide
VDIIDK

SEQ ID NO: 29 - protein, 6 aa, Artificial Sequence
  Capping peptide
EDIINK

SEQ ID NO: 30 - protein, 6 aa, Artificial Sequence
  Capping peptide
DQIFYK

SEQ ID NO: 31 - protein, 6 aa, Artificial Sequence
  Capping peptide
DQIMYK

SEQ ID NO: 32 - protein, 6 aa, Artificial Sequence
  Capping peptide
DQIWYK

SEQ ID NO: 33 - protein, 6 aa, Artificial Sequence
  Capping peptide
YQIYYK

SEQ ID NO: 34 - protein, 6 aa, Artificial Sequence
  Capping peptide
WQIWYK

SEQ ID NO: 35 - protein, 6 aa, Artificial Sequence
  Capping peptide
WQIYYK

SEQ ID NO: 36 - protein, 14 aa, Artificial Sequence
  Capping peptide
VQWINKGSWQIVYK

SEQ ID NO: 37 - protein, 14 aa, Artificial Sequence
  Capping peptide
VQWINKGSVQIVYP

SEQ ID NO: 38 - protein, 14 aa, Artificial Sequence
  Capping peptide
VQWINKGSVPIVYK

SEQ ID NO: 39 - protein, 14 aa, Artificial Sequence
  Capping peptide
VQWINKGSFQIVYK

SEQ ID NO: 40 - protein, 14 aa, Artificial Sequence
  Capping peptide
VQWINKGSVQMVYK

SEQ ID NO: 41 - protein, 14 aa, Artificial Sequence
  Capping peptide
VDIINKGSWQIVYK

SEQ ID NO: 42 - protein, 14 aa, Artificial Sequence
  Capping peptide
VDIINKGSVQIVYP

SEQ ID NO: 43 - protein, 14 aa, Artificial Sequence
  Capping peptide
VDIINKGSVPIVYK

SEQ ID NO: 44 - protein, 14 aa, Artificial Sequence
  Capping peptide
VDIINKGSFQIVYK

SEQ ID NO: 45 - protein, 14 aa, Artificial Sequence
  Capping peptide
VDIINKGSVQMVYK

SEQ ID NO: 46 - protein, 14 aa, Artificial Sequence
  Capping peptide
VQIIDKGSVQIVYP

SEQ ID NO: 47 - protein, 14 aa, Artificial Sequence
  Capping peptide
VQIIDKGSVPIVYK

SEQ ID NO: 48 - protein, 14 aa, Artificial Sequence
  Capping peptide
VQIIDKGSFQIVYK

SEQ ID NO: 49 - protein, 14 aa, Artificial Sequence
  Capping peptide
VQIIDKGSVQMVYK

SEQ ID NO: 50 - protein, 14 aa, Artificial Sequence
  Capping peptide
EQIINKGSWQIVYK

SEQ ID NO: 51 - protein, 14 aa, Artificial Sequence
  Capping peptide
EQIINKGSVQIVYP

SEQ ID NO: 52 - protein, 14 aa, Artificial Sequence
  Capping peptide
EQIINKGSVPIVYK

SEQ ID NO: 53 - protein, 14 aa, Artificial Sequence
  Capping peptide
EQIINKGSFQIVYK

SEQ ID NO: 54 - protein, 14 aa, Artificial Sequence
  Capping peptide
EQIINKGSVQMVYK

SEQ ID NO: 55 - protein, 14 aa, Artificial Sequence
  Capping peptide
VQIINGGSVQIVYP

SEQ ID NO: 56 - protein, 14 aa, Artificial Sequence
  Capping peptide
VQIINGGSVPIVYK

SEQ ID NO: 57 - protein, 14 aa, Artificial Sequence
  Capping peptide
VQIINGGSFQIVYK

SEQ ID NO: 58 - protein, 6 aa, Artificial Sequence
  APR
MVGGVV

SEQ ID NO: 59 - protein, 6 aa, Artificial Sequence
  APR
GGVVIA

SEQ ID NO: 60 - protein, 6 aa, Artificial Sequence
  APR
KLVFFA

SEQ ID NO: 61 - protein, 6 aa, Artificial Sequence
  APR
KLVFFA

SEQ ID NO: 62 - protein, 6 aa, Artificial Sequence
  APR
GAIIGL

SEQ ID NO: 63 - protein, 14 aa, Artificial Sequence
  Capping peptide
MVWGVVGSGWVVIA

SEQ ID NO: 64 - protein, 14 aa, Artificial Sequence
  Capping peptide
MVWGVVGSKLKFFA

SEQ ID NO: 65 - protein, 14 aa, Artificial Sequence
  Capping peptide
MVWGVVGSKLWFFA

SEQ ID NO: 66 - protein, 14 aa, Artificial Sequence
  Capping peptide
MVWGVVGSKWVFFA

SEQ ID NO: 67 - protein, 14 aa, Artificial Sequence
  Capping peptide
MVWGVVGSKWVFFP

SEQ ID NO: 68 - protein, 14 aa, Artificial Sequence
  Capping peptide
MVWGVVGSGAPIGL

SEQ ID NO: 69 - protein, 14 aa, Artificial Sequence
  Capping peptide
MVGGVKGSGWVVIA

SEQ ID NO: 70 - protein, 14 aa, Artificial Sequence
  Capping peptide
MVGGVKGSKLKFFA

SEQ ID NO: 71 - protein, 14 aa, Artificial Sequence
  Capping peptide
MVGGVKGSKLWFFA

SEQ ID NO: 72 - protein, 14 aa, Artificial Sequence
  Capping peptide
MVGGVKGSKWVFFA

SEQ ID NO: 73 - protein, 14 aa, Artificial Sequence
  Capping peptide
MVGGVKGSKWVFFP

SEQ ID NO: 74 - protein, 14 aa, Artificial Sequence
  Capping peptide
MVGGVKGSGAPIGL

SEQ ID NO: 75 - protein, 14 aa, Artificial Sequence
  Capping peptide
MVGGPVGSGWVVIA

SEQ ID NO: 76 - protein, 14 aa, Artificial Sequence
  Capping peptide
MVGGPVGSKLKFFA

SEQ ID NO: 77 - protein, 14 aa, Artificial Sequence
  Capping peptide
MVGGPVGSKLWFFA

SEQ ID NO: 78 - protein, 14 aa, Artificial Sequence
  Capping peptide
MVGGPVGSKWVFFA

SEQ ID NO: 79 - protein, 14 aa, Artificial Sequence
  Capping peptide
MVGGPVGSKWVFFP

SEQ ID NO: 80 - protein, 14 aa, Artificial Sequence
  Capping peptide
MVGGPVGSGAPIGL

SEQ ID NO: 81 - protein, 14 aa, Artificial Sequence
  Capping peptide
GWVVIAGSKLKFFA

SEQ ID NO: 82 - protein, 14 aa, Artificial Sequence
  Capping peptide
GWVVIAGSKLWFFA

SEQ ID NO: 83 - protein, 14 aa, Artificial Sequence
  Capping peptide
GWVVIAGSKLWFFA

SEQ ID NO: 84 - protein, 14 aa, Artificial Sequence
  Capping peptide
GWVVIAGSKWVFFP

SEQ ID NO: 85 - protein, 14 aa, Artificial Sequence
  Capping peptide
GWVVIAGSGAPIGL

SEQ ID NO: 86 - protein, 14 aa, Artificial Sequence
  Capping peptide
KLKFFAGSGAPIGL

SEQ ID NO: 87 - protein, 14 aa, Artificial Sequence
  Capping peptide
KLWFFAGSGAPIGL

SEQ ID NO: 88 - protein, 14 aa, Artificial Sequence
  Capping peptide
KWVFFAGSGAPIGL

SEQ ID NO: 89 - protein, 14 aa, Artificial Sequence
  Capping peptide
KWVFFPGSGAPIGL

SEQ ID NO: 90 - protein, 6 aa, Artificial Sequence
  Capping peptide
KLWFFA

SEQ ID NO: 91 - protein, 6 aa, Artificial Sequence
  Capping peptide
MVWGVV

SEQ ID NO: 92 - protein, 11 aa, Artificial Sequence
  APR
YTIAALLSPYS

SEQ ID NO: 93 - protein, 11 aa, Artificial Sequence
  Capping peptide
YTIYALLSPYS

SEQ ID NO: 94 - protein, 11 aa, Artificial Sequence
  Capping peptide
YTIAPLLSPYS

SEQ ID NO: 95 - protein, 11 aa, Artificial Sequence
  Capping peptide
YTIAALFSPYS

SEQ ID NO: 96 - protein, 6 aa, Artificial Sequence
  APR
LYQLEN

SEQ ID NO: 97 - protein, 7 aa, Artificial Sequence
  APR
LVEALYL

SEQ ID NO: 98 - protein, 6 aa, Artificial Sequence
  Capping peptide
LYYLEN

SEQ ID NO: 99 - protein, 6 aa, Artificial Sequence
  Capping peptide
LYWLEN

SEQ ID NO: 100 - protein, 6 aa, Artificial Sequence
  Capping peptide
SYQLEN

SEQ ID NO: 101 - protein, 7 aa, Artificial Sequence
  Capping peptide
LVEASYL

SEQ ID NO: 102 - protein, 7 aa, Artificial Sequence
  Capping peptide
LVYALYL

SEQ ID NO: 103 - protein, 7 aa, Artificial Sequence
  Capping peptide
LVEALYL

SEQ ID NO: 104 - protein, 6 aa, Artificial Sequence
  APR
AILSST

SEQ ID NO: 105 - protein, 7 aa, Artificial Sequence
  APR
NVGSNTY

SEQ ID NO: 106 - protein, 6 aa, Artificial Sequence
  APR
SSTNVG

SEQ ID NO: 107 - protein, 7 aa, Artificial Sequence
  APR
NFGAILS

SEQ ID NO: 108 - protein, 6 aa, Artificial Sequence
  APR
ANFLVH

SEQ ID NO: 109 - protein, 6 aa, Artificial Sequence
  APR
LVHSSN

SEQ ID NO: 110 - protein, 6 aa, Artificial Sequence
  Capping peptide
AIPSST

SEQ ID NO: 111 - protein, 6 aa, Artificial Sequence
  Capping peptide
AILSSF

SEQ ID NO: 112 - protein, 6 aa, Artificial Sequence
  Capping peptide
AILSPT

SEQ ID NO: 113 - protein, 7 aa, Artificial Sequence
  Capping peptide
NVGSLTY

SEQ ID NO: 114 - protein, 7 aa, Artificial Sequence
  Capping peptide
EVGSNTY

SEQ ID NO: 115 - protein, 7 aa, Artificial Sequence
  Capping peptide
NVGSNGY

SEQ ID NO: 116 - protein, 6 aa, Artificial Sequence
  Capping peptide
SKTNVG

SEQ ID NO: 117 - protein, 6 aa, Artificial Sequence
  Capping peptide
SSTNVE

SEQ ID NO: 118 - protein, 6 aa, Artificial Sequence
  Capping peptide
SSTNVW

SEQ ID NO: 119 - protein, 7 aa, Artificial Sequence
  Capping peptide
NFGFILS

SEQ ID NO: 120 - protein, 7 aa, Artificial Sequence
  Capping peptide
NFGRILS

SEQ ID NO: 121 - protein, 7 aa, Artificial Sequence
  Capping peptide
NFGEILS

SEQ ID NO: 122 - protein, 6 aa, Artificial Sequence
  Capping peptide
WNFLVH

SEQ ID NO: 123 - protein, 6 aa, Artificial Sequence
  Capping peptide
ANWLVH

SEQ ID NO: 124 - protein, 6 aa, Artificial Sequence
  Capping peptide
ANFLRH

SEQ ID NO: 125 - protein, 6 aa, Artificial Sequence
  Capping peptide
LVPSSN

SEQ ID NO: 126 - protein, 6 aa, Artificial Sequence
  Capping peptide
WVHSSN

SEQ ID NO: 127 - protein, 6 aa, Artificial Sequence
  Capping peptide
LVHGSN

SEQ ID NO: 128 - protein, 6 aa, Artificial Sequence
  APR
LSFSKD

SEQ ID NO: 129 - protein, 6 aa, Artificial Sequence
  Capping peptide
LSFPKD

SEQ ID NO: 130 - protein, 6 aa, Artificial Sequence
  Capping peptide
MSFSKD

SEQ ID NO: 131 - protein, 6 aa, Artificial Sequence
  Capping peptide
LSFSED

SEQ ID NO: 132 - protein, 6 aa, Artificial Sequence
  APR
GGVLVN

SEQ ID NO: 133 - protein, 6 aa, Artificial Sequence
  Capping peptide
GRVLVN

SEQ ID NO: 134 - protein, 6 aa, Artificial Sequence
  Capping peptide
GWVLVN

SEQ ID NO: 135 - protein, 6 aa, Artificial Sequence
  Capping peptide
GKVLVN

SEQ ID NO: 136 - protein, 7 aa, Artificial Sequence
  APR
DSVISLS

SEQ ID NO: 137 - protein, 7 aa, Artificial Sequence
  APR
GVIGIAQ

SEQ ID NO: 138 - protein, 7 aa, Artificial Sequence
  Capping peptide
DSRISLS

SEQ ID NO: 139 - protein, 7 aa, Artificial Sequence
  Capping peptide
DSVISLP

SEQ ID NO: 140 - protein, 7 aa, Artificial Sequence
  Capping peptide
QSVISLS

SEQ ID NO: 141 - protein, 7 aa, Artificial Sequence
  Capping peptide
GVIWIAQ

SEQ ID NO: 142 - protein, 7 aa, Artificial Sequence
  Capping peptide
GVIGIPQ

SEQ ID NO: 143 - protein, 7 aa, Artificial Sequence
  Capping peptide
GVIYIAQ

SEQ ID NO: 144 - protein, 6 aa, Artificial Sequence
  APR
IFQINS

SEQ ID NO: 145 - protein, 6 aa, Artificial Sequence
  Capping peptide
IFQIES

SEQ ID NO: 146 - protein, 6 aa, Artificial Sequence
  Capping peptide
IFQIDS

SEQ ID NO: 147 - protein, 6 aa, Artificial Sequence
  Capping peptide
IFQIFS

SEQ ID NO: 148 - protein, 11 aa, Artificial Sequence
  APR
GAVVTGVTAVA

SEQ ID NO: 149 - protein, 11 aa, Artificial Sequence
  Capping peptide
GAVVTGVTAVF

SEQ ID NO: 150 - protein, 11 aa, Artificial Sequence
  Capping peptide
GAVVTGVTAVA

SEQ ID NO: 151 - protein, 11 aa, Artificial Sequence
  Capping peptide
GAVVTGVTAVA

SEQ ID NO: 152 - protein, 7 aa, Artificial Sequence
  APR
LTIITLE

SEQ ID NO: 153 - protein, 7 aa, Artificial Sequence
  Capping peptide
LTIITYE

SEQ ID NO: 154 - protein, 7 aa, Artificial Sequence
  Capping peptide
LTIITLE

SEQ ID NO: 155 - protein, 7 aa, Artificial Sequence
  Capping peptide
LPIITLE

SEQ ID NO: 156 - protein, 7 aa, Artificial Sequence
  APR
GGYMLGS

SEQ ID NO: 157 - protein, 7 aa, Artificial Sequence
  APR
GGYVLGS

SEQ ID NO: 158 - protein, 7 aa, Artificial Sequence
  APR
GYLLGSA

SEQ ID NO: 159 - protein, 7 aa, Artificial Sequence
  Capping peptide
LGYMLGS

SEQ ID NO: 160 - protein, 7 aa, Artificial Sequence
  Capping peptide
GGYMLVS

SEQ ID NO: 161 - protein, 7 aa, Artificial Sequence
  Capping peptide
KGYMLGS

SEQ ID NO: 162 - protein, 7 aa, Artificial Sequence
  Capping peptide
GGYVYGS

SEQ ID NO: 163 - protein, 7 aa, Artificial Sequence
  Capping peptide
GGYRLGS

SEQ ID NO: 164 - protein, 7 aa, Artificial Sequence
  Capping peptide
GGYVFGS

SEQ ID NO: 165 - protein, 7 aa, Artificial Sequence
  Capping peptide
GYLLHSA

SEQ ID NO: 166 - protein, 7 aa, Artificial Sequence
  Capping peptide
GYLLYSA

SEQ ID NO: 167 - protein, 7 aa, Artificial Sequence
  Capping peptide
GYLLFSA

SEQ ID NO: 168 - protein, 10 aa, Artificial Sequence
  APR
GVVHGVTTVA

SEQ ID NO: 169 - protein, 10 aa, Artificial Sequence
  Capping peptide
GVVHGVTTRA

SEQ ID NO: 170 - protein, 10 aa, Artificial Sequence
  Capping peptide
GVVHGVETVA

SEQ ID NO: 171 - protein, 10 aa, Artificial Sequence
  Capping peptide
GVVHMVTTVA

SEQ ID NO: 172 - protein, 5 aa, Artificial Sequence
  APR
YTFGQ

SEQ ID NO: 173 - protein, 5 aa, Artificial Sequence
  Capping peptide
YPFGQ

SEQ ID NO: 174 - protein, 5 aa, Artificial Sequence
  Capping peptide
YLFGQ

SEQ ID NO: 175 - protein, 5 aa, Artificial Sequence
  Capping peptide
NTFGQ

SEQ ID NO: 176 - protein, 6 aa, Artificial Sequence
  target TDP-43
GNNSYS

SEQ ID NO: 177 - protein, 6 aa, Artificial Sequence
  Capping peptide
GNNSYY

SEQ ID NO: 178 - protein, 6 aa, Artificial Sequence
  Capping peptide
GNNHYS

SEQ ID NO: 179 - protein, 6 aa, Artificial Sequence
  Capping peptide
GNNSYF

SEQ ID NO: 180 - protein, 8 aa, Artificial Sequence
  APR
SYSSYGQS

SEQ ID NO: 181 - protein, 8 aa, Artificial Sequence
  Capping peptide
SYSSYWQS

SEQ ID NO: 182 - protein, 8 aa, Artificial Sequence
  Capping peptide
SYSSYYQS

SEQ ID NO: 183 - protein, 8 aa, Artificial Sequence
  Capping peptide
SYSSYIQS

SEQ ID NO: 184 - protein, 9 aa, Artificial Sequence
  Ligand
DDDWTHLSS

SEQ ID NO: 185 - protein, 9 aa, Artificial Sequence
  Ligand
SEDYIIILP

SEQ ID NO: 186 - protein, 9 aa, Artificial Sequence
  Ligand
NSDMLVVTT

(19) United States
(12) Patent Application Publication
(10) Pub. No.: US 2023/0181748 A1
(43) Pub. Date: Jun. 15, 2023

(54) MEANS AND METHODS FOR THE TREATMENT OF PATHOLOGICAL AGGREGATION

(71) Applicants: VIB VZW, Gent (BE); KATHOLIEKE UNIVERSITEIT LEUVEN, K.U.LEUVEN R&D, Leuven (BE)

(72) Inventors: Joost Schymkowitz, Meensel-Kiezegem (BE); Frederic Rousseau, Sint-Martens-Bodegem (BE); Emiel Michiels, Leuven (BE); Nikolaos Louros, Kessel-Lo (BE)

(21) Appl. No.: 17/925,160

(22) PCT Filed: May 17, 2021
(86) PCT No.: PCT/EP2021/063001

§ 371 Date: Nov. 14, 2022

(30) Foreign Application Priority Data
May 15, 2020 (EP) 20174994.2

(51) Int. Cl. A61K 47/64; A61K 47/54; G01N 33/68

(52) U.S. Cl. CPC A61K 47/64; A61K 47/54; G01N 33/6845

(57) ABSTRACT

The present invention provides non-natural molecules which comprise a peptide part able to stop the amyloid aggregation which is fused to a moiety which stimulates the proteasomal degradation pathway in the cell. Non-natural molecules of the invention are useful to treat human and veterinary pathological aggregation disorders.

### CROSS-REFERENCE TO RELATED APPLICATIONS

This application is a national phase entry under 35 U.S.C. § 371 of International Patent Application PCT/EP2021/063001, filed May 17, 2021, designating the United States of America and published in English as International Patent Publication WO 2021/229102 on Nov. 18, 2021, which claims the benefit under Article 8 of the Patent Cooperation Treaty to European Patent Application Serial No. 20174994.2, filed May 15, 2020, the entireties of which are hereby incorporated by reference.

### FIELD OF THE INVENTION

The present invention relates to the field of protein aggregation, more particularly to the field of pathological protein aggregation, particularly pathological amyloid and non-amyloid aggregation. Even more particularly the invention belongs to the field of pathological aggregation such as for example aggregates of amyloid-beta, tau, alpha-synuclein, TDP-43, p53, FUS and the like. Specifically, the present invention provides non-natural molecules which comprise a peptide part able to prevent or stop the pathological aggregation which is fused to a moiety which stimulates a proteolytic degradation pathway in the cell. Non-natural molecules of the invention are useful to treat or prevent human and veterinary pathological aggregation disorders.

### INTRODUCTION TO THE INVENTION

Pathological aggregates result from the association of multiple individual peptide units into large clusters. Such pathological aggregates are generally categorized into i) amyloid structures which can be further divided into amyloid fibrils and amyloid oligomers and ii) non-amyloid structures. Amyloid fibrils are mainly composed of beta-sheets and share common characteristics, including a cross-beta X-ray diffraction pattern and characteristic staining by the dye Congo Red. The formation of these fibrils resembles a crystallization in one dimension. Short peptides containing the key sequences of 5 to 10 residues necessary for the formation of several amyloids have been crystallized and subsequently used to seed amyloid formation from the relevant proteins (Sawaya MR et al (2007) Nature 447, 453-457). Amyloid oligomers are mainly associated intracellularly and are considered predecessors of amyloid fibrils and are considered as more toxic than mature amyloid fibrils. Amyloid and non-amyloid structures are associated with several diseases, either as a symptom but mostly as a cause for the disease. For example, amyloid diseases are associated with the transformation of normally soluble proteins into amyloid structures such as fibrils and oligomers. Interestingly to date, 37 different peptides or proteins have been found to form amyloid oligomers or to form amyloid intracellular or extracellular deposits in human pathologies (see Table 1 in Chiti F and Dobson C M (2017) Annu. Rev. Biochem. 86: 27-68). Many of these amyloids are secreted, and the deposits are available in the extracellular space, while other amyloids are cytosolic and form intracellular inclusions with amyloid-like characteristics. Amyloid oligomers are the progenitors of the amyloid fibrils and are most often found intracellularly. Indeed, two of the most studied extracellular amyloid fibrils amyloid beta and IAPP have also been found intracellularly, e.g. as oligomers. In general polypeptides able to form amyloid structures are small in size. Indeed, half of them have fewer than 100 amino acid residues, only 4 have more than 400 residues, and none has more than 700 residues while the average length of the more than 30,000 proteins encoded in the human genome is about 500 residues. Seven of the known proteins associated with amyloid disease form oligomers and deposits in the central nervous system, giving rise to neurodegenerative conditions, such as Alzheimer's and Parkinson's disease, whereas the remainder form oligomers and deposits in other tissues (including the heart, spleen, liver and kidney), and the resulting diseases are therefore non-neuropathic (see Table 1 in Chiti F and Dobson CM (2017) Annu. Rev. Biochem. 86: 27-68). In Alzheimer's disease, two different kinds of misfolding/aggregation occur: i) plaques of aggregated beta-sheet-like proteins (mainly consisting of amyloid beta peptide), and ii) the protein, tau, which normally promotes aggregation of tubulin to form the natural tubular material of neuron fibrils, instead forms other aggregates, which results in neurons' losing their neuron fibrils and, thus their function. Transthyretin is another example and the resulting amyloid disease is generally systemic or involves the peripheral nervous system or the heart. Also to date, 19 peptides or proteins are described which can form intracellular or extracellular non-amyloid deposits in human diseases (see Table 2 in Chiti F and Dobson CM (2017) Annu. Rev. Biochem. 86: 27-68).

Because of the association of pathological aggregates (e.g. collectively formed by amyloid or non-amyloid extracellular or intracellular deposits) with disease, there have been several attempts at delaying and preventing pathological aggregate formation. The ability of antibodies to bind not only to unique sequences but also to well-defined aggregation states has led to considerable efforts to develop immunotherapies for pathological aggregation diseases (see for example Briggs R et al (2016) Clin. Med. 16:247-53). Unfortunately, all attempts to develop pathological aggregation inhibitors have failed at an early or later stage of clinical trials. Examination of the failures indicate that the lack of methods to monitor the aggregation reaction in a reliable manner, the lack of knowledge of the mechanism of action of the compounds and the impossibility to intervene early in the disease are contributing factors. Others have focused on the screening of compounds in vitro and in vivo to identify potential inhibitors targeted towards neurodegenerative diseases. Nevertheless, compounds having a peptide structure (including non-natural amino acids and/or D-amino acids) that inhibit aggregation of a target peptide are well-known in the art and such peptides are designated as capping peptides. Examples are e.g. U.S. Pat. No. 8,754,034B2 for tau aggregation inhibitors, WO2018/005866 for transthyretin inhibitors, US2019/0241613 for alpha-synuclein inhibitors. These specific capping peptides efficiently prevent the further growth of initial aggregates of specific pathological proteins, but these capping peptides cannot eliminate the established pathological aggregates, or the established pathological oligomers present in the cell. Efforts to clean-up pathological tau aggregates have been disclosed in WO2018/102067 and Silva MC et al 2019) eLIFE, 8, e45457). However, in the latter method bi-functional compounds were used (one part binding on tau and another part binding on an ubiquitin ligase) and these compounds degrade the hyperphosphorylated (monomeric forms of tau) which are a risk factor for pathological aggregation formation.

### SUMMARY OF THE INVENTION

The present invention has generated a new class of non-natural molecules which comprise a capping peptide which can prevent or stop the pathological aggregation of a target protein wherein the capping peptide is fused to a moiety which interacts with one or more polypeptide components of the intracellular proteolysis system. It is shown in the invention that these non-natural molecules can efficiently enter the cells, can prevent the further aggregation of a pathological protein and importantly can also degrade the pathological aggregates formed by said protein. Surprisingly, our non-natural molecules do not interfere with (or do not degrade the) the monomeric forms of the pathological aggregates of the proteins and hence these molecules are specific for degrading only the pathological aggregates (such as amyloid and non-amyloid aggregates).

Accordingly, the present invention provides in one aspect a non-natural molecule comprising at least one capping peptide specifically binding to a pathological aggregate of a target protein capable of forming pathological aggregates wherein said at least one capping peptide is fused to at least one moiety targeting the intracellular proteolytic degradation system.

In another aspect, a non-natural molecule of the present invention provides an non-natural molecule comprising at least two capping peptides specifically binding to a pathological amyloid or non-amyloid aggregate of a target protein capable of forming pathological aggregates wherein the at least 2 capping peptides are directed to the same or different aggregation prone regions of the target protein and wherein the capping peptides are fused to at least one moiety targeting the intracellular proteolytic degradation system. The wording “directed to” is equivalent with “binds with”.

In yet another aspect the intracellular proteolytic degradation system is the ubiquitin proteasome degradation system (UPS).

In yet another aspect the intracellular proteolytic degradation system is the autophagy degradation system.

In yet another aspect the intracellular proteolytic degradation system is the chaperone-mediated autophagy (CMA) system.

In a specific aspect the moiety targeting the intracellular proteolytic degradation system is a small molecule.

In yet another specific aspect the moiety targeting the intracellular proteolytic degradation system is a peptide or has a peptide-like structure.

A peptide-like structure is a peptide wherein one or more amino acids of the peptide sequence are changed into D-amino acids or into artificial amino acids.

In a specific aspect the non-natural molecule targets an intracellular pathological aggregate such as an amyloid fibril or an amyloid oligomer or a non-amyloid aggregate such as p53, FUS or TDP-43.

In another specific aspect the non-natural molecule targets an extracellular pathological aggregate

In another aspect the non-natural molecule targets an intracellular amyloid forming protein such as for example tau, IAPP amyloid-beta, alfa-synuclein, huntingtin-1.

In another aspect the non-natural molecule targets an intracellular non-amyloid forming protein such as for example ataxin-1, FUS, TDP-43 and p53.

In yet another aspect the non-natural molecule further comprises a half-life extension entity.

In yet another aspect the non-natural molecule further comprises a cell penetrating entity.

In yet another aspect the non-natural molecule further comprises one or more artificial amino acids.

In yet another aspect the non-natural molecules of the invention are provided for use as a medicament.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1: schematic presentation of the requirements to be fulfilled by a candidate capping peptide (for details see example 1).

FIG. 2: interaction energies of candidate capping peptides for the APR derived from a tau (depicted in SEQ ID NO: 8). The X-axis represents the cross-interaction energy and the Y-axis represents the elongation energy. Suitable tau candidate capping peptides are situated in the left-upper corner (aggregation capping).

FIGS. 3A-3F: An illustration of the tau biosensor seeding assay. Cells were transfected with atto-633 labeled preformed tau aggregates that were pre-treated with buffer (FIGS. 3A and 3B), a non-fused capping peptide (FIGS. 3C and 3D) or a non-natural molecule (capping peptide coupled to a degradation moiety (FIGS. 3E and 3F)). Green spots represent induced endogenous tau aggregates (FIG. 3A) and red spots are the exogenous preformed tau aggregates (FIG. 3B). Cells solely exposed to preformed tau aggregates show induced tau aggregation (FIG. 3A) and are positive for exogenous preformed tau aggregates (FIG. 3B). Cells exposed to preformed tau aggregates that were pre-treated with a capping peptide show reduced tau aggregation (FIG. 3C) but are still positive for exogenous preformed tau aggregates (FIG. 3D). Cells exposed to preformed tau aggregates pre-treated with a capping-degrader peptide show reduced tau aggregation (FIG. 3E) and a clear reduction in exogenous preformed tau aggregates (FIG. 3F). The FIGS. 3A, 3C and 3E represent the green spots of the same cells with red spots in FIGS. 3B, 3D and 3F, respectively.

FIG. 4: Screening for reduced tau aggregation in the tau biosensor seeding assay. Cells were transfected with atto-633 labeled preformed tau aggregates pre-treated with buffer (CTRL) or a specific capping (-degrader) peptide. Two independent repeats are shown ±SD of the fraction of cells that show green spots (induced tau aggregation).

FIG. 5: Screening for tau aggregate degradation in the tau biosensor seeding assay. Cells were transfected with atto-633 labeled preformed tau aggregates pre-treated with buffer (CTRL) or a specific capping (-degrader) peptide. Two independent repeats are shown ±SD of the fraction of cells that show red spots (exogenous tau aggregates).

FIGS. 6A and 6B: The effect of a concentration range of peptide CAP1_TR and the non-natural molecules (degrader variants) on induced tau aggregation and tau aggregate degradation. (FIG. 6A) The fraction of cells that show green spots (induced tau aggregation). The mean value of at least three independent repeats is shown ±SD. (FIG. 6B) The fraction of cells that show red spots (exogenous tau aggregates). The mean value of at least three independent repeats is shown ±SD.

FIGS. 7A and 7B: The effect of a concentration range of peptide HET and the non-natural molecules derived thereof comprising the degradation moieties (degrader variants) on induced tau aggregation and tau aggregate degradation. FIG. 7A depicts the fraction of cells that have green spots (induced tau aggregation). The mean value of at least three independent repeats is shown ±SD. FIG. 7B depicts the fraction of cells that have red spots (exogenous tau aggregates). The mean value of at least three independent repeats is shown ±SD.

FIG. 8: The effect of different capping peptides and non-natural molecules derived thereof on tau biosensor cells seeded with preformed tau aggregates. Preformed tau aggregates were incubated with buffer (CTRL) or a capping peptide (CAP1_TR and HET) and non-natural molecules comprising the capping peptides (CAP1_TR-JAV and HET-JAV) for 6 hours prior to cell transfection. Final peptide concentration on the cells of the capping peptides and non-natural molecules was 312 nM).

FIGS. 9A and 9B: The effect of the capping peptide CAP1_TR and the non-natural molecules derived thereof on in vitro tau aggregation. (FIG. 9A) Monomeric tau (9 μM) was aggregated without (grey curves) or with the addition of capping peptides and non-natural molecules derived thereof (6 μM, black curves). Th-T was added to monitor aggregation. At least two independent repeats are shown. (FIG. 9B) Monomeric tau (9 μM) was aggregated with preformed tau aggregates (grey curves) or with preformed tau aggregates that were pretreated with capping peptides and non-natural molecules derived thereof (6 μM, black curves). Th-T was added to monitor aggregation. At least two independent repeats are shown.

FIGS. 10A and 10B: The effect of capping peptide HET and the non-natural molecules derived thereof on in vitro tau aggregation. (FIG. 10A) Monomeric tau (9 μM) was aggregated without (grey curves) or with the addition of capping peptides and non-natural molecules derived thereof (6 μM, black curves). Th-T was added to monitor aggregation. At least two independent repeats are shown. (FIG. 10B) Monomeric tau (9 μM) was aggregated with preformed tau aggregates (grey curves) or with preformed tau aggregates that were pretreated with capping peptides and non-natural molecules derived thereof (6 μM, black curves). Th-T was added to monitor aggregation. At least two independent repeats are shown.

FIGS. 11A and 11B: Microscale Thermophoresis (MST) experiments for CAP1_TR, HET, and the non-natural molecules comprising specific degradation moieties, with tau monomers and tau aggregates (seeds). Capping peptides and non-natural molecules derived thereof were incubated with monomeric tau (grey) or preformed tau aggregates (black) and ˜15 minutes after mixing, MST values were collected. Data represent normalized mean values of 9 replicates (3 independent) ±SD.

FIGS. 12A and 12B: non-natural molecule uptake efficiency in HEK293T cells. Cells were treated with a final concentration of 5 μM of each non-natural molecule or with a buffer control (CTRL) and incubated for 20 hours. (FIG. 12A) An illustration of the uptake of non-natural molecules CAP1_TR-pomalidomide and HET-pomalidomide. (FIG. 12B) The quantification of the fraction of cells positive for pomalidomide-labeled peptide. Three independent replicates are shown ±SD.

FIGS. 13A-13C: The effect of HET and CAP1_TR and the non-natural molecules (degrader variants) on monomeric tau levels in the tau biosensor cell line. (FIG. 13A) Cells were transfected with a final concentration of 2.5 μM of each peptide and incubated for 20 hours before cell fixation and imaging. (FIGS. 13B and 13C) Total tau levels, monitored by measuring tau-CFP fluorescence, normalized to buffer-treated (CTRL) cells, following transfection with 2.5 μM (FIG. 13B) or 313 nM (FIG. 13C) capping (degrader) peptide. The mean value of three independent repeats is shown ±SD.

FIGS. 14A and 14B: Cells exposed to preformed tau aggregates pre-treated with respectively HET, HET-JAV, HET-CMA1 and HET-CMA2 show a reduced tau aggregation (FIG. 14A) and a reduction in exogenous preformed tau aggregates (FIG. 14B). An outline of this experiment is described in example 2.

FIG. 15: Hetero-dimeric tandem capping peptide screen. Capping peptides were dissolved in DMSO, mixed with sonicated preformed Tau aggregates or Sup35-NM aggregates and transfected into a Tau biosensor cell line, expressing CFP- and YFP- labeled Tau repeat domain and a NM biosensor cell line, expressing GFP-labeled Sup35-NM. The final concentration of capping peptide on cells was 625 nM and the final concentration of aggregates (Tau or NM) was 50 nM. After 24 hours incubation, the fraction of cells with induced aggregates was determined and normalized. The condition is which cells were treated with preformed aggregates without capping peptide was set at 1, while the condition in which cells were not treated with preformed aggregates was set at 0. The results show the average of 3 independent repeats (each consisting of 3 technical replicates).

FIGS. 16A-16C: Amyloid beta capping peptide design. FoldX was used to calculate the cross interaction and elongation energy of every single amino acid mutant of the corresponding amyloid beta APR with the growing (wild type) amyloid chain. 2okz and Zona are the identification numbers present in the protein structure database (https:www.uniprot.org) for the APR sequence MVGGVV (SEQ ID NO: 58), 2onv is the entry for the structure of the APR sequence GGVVIA (SEQ ID NO: 59), 2y2a is the entry for the structure of the APR sequence KLVFFA (SEQ ID NO: 60), 2y29 is the entry for the structure of the APR sequence KLVFFA (SEQ ID NO: 61) and 3ppz is the entry for the structure of the APR sequence GAIIGL (SEQ ID NO: 62). The plots only show the single mutants with a negative cross interaction energy and a positive elongation energy, the latter are candidate capping peptides for inhibiting aggregation of amyloid beta pathological aggregates.

FIG. 17: Aβ biosensor screening assay. Aβ biosensor cells stably express soluble Aβ-mCherry (No Aβ seeds) and Aβ aggregation can be induced by transfecting preformed Aβ aggregates in these cells (100 nM Aβ seeds). Adding capping peptides to the Aβ seeds reduces seeding capacity of Aβ seeds (100 nM Aβ seeds +capping peptide).

FIG. 18: Aβ biosensor screening assay of the first batch of hetero-tandem capping peptides. Quantification of the relative number of cells positive for induced Aβ aggregation. Data are normalized to a condition in which no seeds are added (0) and a condition in which 100 nM Aβ aggregates are added without capping peptides (1). The mean of 2 independent repeats is shown ±SD.

FIGS. 19A and 19B: Aβ biosensor screening assay of the second batch of hetero-tandem capping peptides. Quantification of the relative number of cells positive for induced Aβ aggregation. Data are normalized to a condition in which no seeds are added (0) and a condition in which 100 nM Aβ aggregates are added without capping peptides (1). The mean of 3 independent repeats is shown ±SD.

FIG. 20: Aβ biosensor screening assay of the second batch of hetero-tandem capping peptides. Visualization of the concentration-dependent effect of AbCap 4 on the seeding capacity of Aβ seeds.

FIG. 21: Viability of cells in the Aβ biosensor screening assay of the second batch of hetero-tandem capping peptides. Relative cell viability (compared to non-treated cells) for all concentrations of capping peptides.

FIGS. 22A-22D: Analysis of hetero-capping activity to identify dominant single capping sequences. Each graph represents the data for one concentration of hetero-capping peptide. Data represent the quantification of the relative number of cells positive for induced Aβ aggregation. Data are normalized to a condition in which no seeds are added (0) and a condition in which 100 nM Aβ aggregates are added without capping peptides (1). Each data points represents the average of at least three independent repeats of one peptide. Hence, every dot represents one hetero-tandem peptide. As every hetero-tandem peptide consists of two single capping sequences, each hetero-tandem peptide is represented by two dots.

FIG. 23: Illustration of the hit validation assay with Aβ-647 seeds. Images represent the state of the cells after 20 hours of incubated with 500nM Aβ-647 seeds that were pre-treated with hetero-capping peptides (final concentration =625 nM). Red channel (upper panel of each AbCap example) shows Aβ-647 seeds, while the orange channel (middel panel of each AbCap example) shows the exogenous Aβ-mCherry. The lower panel of each AbCap example is a merged image of the red channel and the orange channel.

FIGS. 24A and 24B: Aβ biosensor screening assay with Aβ-647 seeds. Quantification of the number of cells positive for induced Aβ aggregation (orange dots) and the number of cells positive for Aβ-647 seeds (red dots). The mean of 3 independent repeats is shown ±SD. Grey and black dotted line represent the relative number of cells positive for induced Aβaggregation and Aβ-647 seeds, respectively, for non-treated cells (no seeds).

FIGS. 25A and 25B: Aβ biosensor screening assay with Aβ-647 seeds (normalized data). Quantification of the number of cells positive for induced Aβ aggregation (orange dots) and the number of cells positive for Aβ-647 seeds (red dots). The mean of 3 independent repeats is shown ±SD. Data are normalized to a condition in which no seeds are added (0) and a condition in which 100 nM Aβ aggregates are added without capping peptides (1), for both the relative number of cells positive for induced Aβ aggregation as well as the relative number of cells positive for Aβ-647 seeds.

FIGS. 26A and 26B: In vitro Aβ seeding aggregation assay. Preformed, sonicated Aβ fibrils were preincubated with capping peptides (grey dots) or buffer (black dots) and used to seed Aβ aggregation. Light grey dots represent Aβ aggregation without addition of preformed, sonicated Aβ fibrils.

FIG. 27: Initial MST experiment of labeled Aβ- and tau fibrils with a single concentration of capping peptide. Values show raw Fnorm data points for each condition.

FIG. 28: MST experiment of labeled Aβ- and tau fibrils with a concentration range of capping peptide. Values represent ΔFnorm data points. Kd values are shown on the graphs (if applicable).

FIG. 29: An amyloid core structure for the target APR sequence YTIAALLSPYS (SEQ ID NO: 92) present in TTR is available in the protein structure database (www.rcsb.org) as 2m5n. The method of generating specific capping peptides based on this amyloid core structure conducted as outlined in example 1 is shown in the figure.

FIG. 30: Capping peptides were designed targeting pathological aggregates of insulin as described in example 1. The amyloid core structure for the target APR sequence LYQLEN (SEQ ID NO: 96), present in insulin, is found in the protein structure database (www.rcsb.org) as 2omp.

FIG. 31: Capping peptides were designed targeting pathological aggregates of insulin as described in example 1. The amyloid core structure for the target APR sequences LVEALYL (SEQ ID NO: 97), present in insulin, is found in the protein structure database (www.rcsb.org) as 3hyd.

FIG. 32: Capping peptides were designed targeting pathological aggregates of IAPP as described in example 1. The amyloid core structure for the APR sequence AILSST (SEQ ID NO: 104) present in IAPP is available in the protein structure database (www.rcsb.org) as 3fod.

FIG. 33: Capping peptides were designed targeting pathological aggregates of IAPP as described in example 1. The amyloid core structure for the APR sequences NVGSNTY (SEQ ID NO: 105) present in IAPP is available in the protein structure database (www.rcsb.org) as 3ft1.

FIG. 34: Capping peptides were designed targeting pathological aggregates of IAPP as described in example 1. The amyloid core structure for the APR sequence SSTNVG (SEQ ID NO: 106) is available in the protein structure database (www.rcsb.org) as 3ftr.

FIG. 35: Capping peptides were designed targeting pathological aggregates of IAPP as described in example 1. The amyloid core structure for the APR sequence NFGAILS (SEQ ID NO: 107) is available in the protein structure database (www.rcsb.org) as 5e5v.

FIG. 36: Capping peptides were designed targeting pathological aggregates of IAPP as described in example 1. The amyloid core structure for the APR sequence ANFLVH (SEQ ID NO: 108) is available in the protein structure database (www.rcsb.org) as 5e5x.

FIG. 37: Capping peptides were designed targeting pathological aggregates of IAPP as described in example 1. The amyloid core structure for the APR sequence LVHSSN (SEQ ID NO: 109) is available in the protein structure database (www.rcsb.org) as 5e5z.

FIG. 38: Capping peptides were designed targeting pathological aggregates of beta-2-microglobulin as described in example 1. An amyloid core structure for the target APR sequence LSFSKD (SEQ ID NO: 128) present in beta2-microglobulin is available in the protein structure database (www.rcsb.org) as 31oz.

FIG. 39: Capping peptides were designed targeting pathological aggregates of prostatic acid phosphatase (PAP) as described in example 1. An amyloid core structure for the target APR sequence GGVLVN (SEQ ID NO: 132) present in PAP is available in the protein structure database (www.rcsb.org) as 3ppd.

FIG. 40: Capping peptides were designed targeting pathological aggregates of SOD1 as described in example 1. An amyloid core structure for the target APR sequences DSVISLS (SEQ ID NO: 136) present in SOD1 is available in the protein structure database (www.rcsb.org) as 4nin.

FIG. 41: Capping peptides were designed targeting pathological aggregates of SOD1 as described in example 1. An amyloid core structures for the target APR sequences GVIGIAQ (SEQ ID NO: 137) present in SOD1 is available in the protein structure database (www.rcsb.org) as 4nip.

FIG. 42: Capping peptides were designed targeting pathological aggregates of lysozyme as described in example 1. An amyloid core structure for the target APR sequence IFQINS (SEQ ID NO: 144) present in lysozyme is available in the protein structure database (www.rcsb.org) as 4r0p.

FIG. 43: Capping peptides were designed targeting pathological aggregates of alfa-synuclein as described in example 1. An amyloid core structure for the target APR sequence GAVVTGVTAVA (SEQ ID NO: 148) present in alpha-synuclein is available in the protein structure database (www.rcsb.org) as 4ri1.

FIG. 44: Capping peptides were designed targeting pathological aggregates of p53 as described in example 1. An amyloid core structure for the target APR sequence LTIITLE (SEQ ID NO: 152) present in p53 is available in the protein structure database (www.rcsb.org) as 4rp6.

FIG. 45: Capping peptides were designed targeting pathological aggregates of PrP as described in example 1. An amyloid core structure for the target APR sequence GGYMLGS (SEQ ID NO: 156 present in PrP is available in the protein structure database (www.rcsb.org) as 4w5m.

FIG. 46: Capping peptides were designed targeting pathological aggregates of PrP as described in example 1. An amyloid core structure for the target APR sequences GGYVLGS (SEQ ID NO: 157) present in PrP is available in the protein structure database (www.rcsb.org) as 4w5p.

FIG. 47: Capping peptides were designed targeting pathological aggregates of PrP as described in example 1. An amyloid core structures for the target APR sequence GYLLGSA (SEQ ID NO: 158) present in PrP is available in the protein structure database (www.rcsb.org) as 4w71.

FIG. 48: Capping peptides were designed targeting pathological aggregates of alpha-synuclein (A53T mutant) as described in example 1. An amyloid core structure for the target APR sequence GVVHGVTTVA (SEQ ID NO: 168) present in the mutant alpha-synuclein is available in the protein structure database (www.rcsb.org) as 4znn.

FIG. 49: Capping peptides were designed targeting pathological aggregates of Ig Light Chain Variable Domain as described in example 1. An amyloid core structure for a conserved target APR sequence YTFGQ (SEQ ID NO: 172) (see FIG. 11 in Brumshtein B et al (2018) J. Biol. Chem. 293(51) 19659) present in a light chain immunoglobulin variable domain sequence is available in the protein structure database (www.rcsb.org) as 6diy.

FIG. 50: Capping peptides were designed targeting pathological aggregates of TDP-43 as described in example 1. An amyloid core structure for the target TDP-43 sequence GNNSYS (SEQ ID NO: 176) present in TDP-43 is available in the protein structure database (www.rcsb.org) as 5wia.

FIG. 51: Capping peptides were designed targeting pathological aggregates of FUS as described in example 1. An amyloid core structure for the target APR sequence SYSSYGQS (SEQ ID NO: 180) present in FUS is available in the protein structure database (www.rcsb.org) as 6bxv.

### DETAILED DESCRIPTION OF THE INVENTION

The present invention will be described with respect to particular embodiments and with reference to certain figures but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying figures. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but they may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

The recitation of numerical ranges by endpoints includes all numbers and fractions subsumed within the respective ranges, as well as the recited endpoints. This applies to numerical ranges irrespective of whether they are introduced by the expression “from... to...” or the expression “between... and...” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any 3, 4, 5, or etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

The terms “polypeptide” and “peptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers. This term also includes post-translational modifications of the polypeptide, such as glycosylation, phosphorylation, amidation, oxidation and acetylation. By “recombinant polypeptide” is meant a polypeptide made using recombinant techniques, i.e., through the expression of a recombinant or synthetic polynucleotide. The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product. The term “recombinant host cell”, “engineered cell”, “expression host cell”, “expression host system”, “expression system” or simply “host cell”, as used herein, is intended to refer to a cell into which a recombinant vector and/or chimeric gene construct has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. The term “modulate,” “modulates,” or “modulation” refers to enhancement (e.g. an increase) or inhibition (e.g. a decrease) in the specified level or activity. The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “non-naturally occurring” generally refers to a material or an entity that is not formed by nature or does not exist in nature. Such non-naturally occurring material or entity may be made, synthesised, semi-synthesised, modified, intervened on or manipulated by man using methods described herein or known in the art. By means of an example, the term when used in relation to a peptide may in particular denote that a peptide of an identical amino acid sequence is not found in nature, or if a peptide of an identical amino acid sequence is present in nature, that the non-naturally occurring peptide comprises one or more additional structural elements such as chemical bonds, modifications or moieties which are not included in and thus distinguish the non-naturally occurring peptide from the naturally occurring counterpart. In certain embodiments, the term when used in relation to a peptide may denote that the amino acid sequence of the non-naturally occurring peptide is not identical to a stretch of contiguous amino acids encompassed by a naturally occurring peptide, polypeptide or protein. For avoidance of doubt, a non-naturally occurring peptide may perfectly contain an amino acid stretch shorter than the whole peptide, wherein the structure of the amino acid stretch including in particular its sequence is identical to a stretch of contiguous amino acids found in a naturally occurring peptide, polypeptide or protein.

The term “contact” or grammatical variations thereof as used with respect to a non-natural molecule of the invention and a pathological aggregate refers to bringing the non-natural molecule and the pathological aggregate in sufficiently close proximity to each other for one to exert a biological effect on the other. A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject. By the terms “treat,” “treating,” or “treatment of,” it is intended that the severity of the subject's condition is reduced or at least partially improved or modified and that some alleviation, mitigation or decrease in at least one clinical symptom is achieved. As used herein, a “functional” peptide is one that substantially retains at least one biological activity normally associated with that peptide (e.g. binding to and inhibiting the formation of amyloid fibrils or amyloid oligomers. In particular embodiments, the “functional” peptide substantially retains all of the activities possessed by the unmodified peptide. By “substantially retains” biological activity, it is meant that the peptide retains at least about 20%, 30%, 40%, 50%, 60%, 75%, 85%, 90%, 95%, 97%, 98%, 99%, or more, of the biological activity of the native polypeptide (and can even have a higher level of activity than the native peptide). Biological activities such as oligomer or fibril or non-amyloid degradation activity can be measured using assays described herein and other assays that are well known in the art.

As used herein the term “conservative amino acid substitution” refers to the substitution of an amino acid that is normally present in the peptide sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

An “artificial amino acid” (or “un-natural” amino acid which is equivalent wording) refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, artificial, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991, vol. 202, 301-36. The incorporation of non-natural amino acids into proteins, polypeptides or peptides may be advantageous in a number of different ways. For example, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the molecule and prolonged lifetimes in vivo.

It is well-known in the art that proteins can adopt a multitude of different conformational states within a living system, between its synthesis on the ribosome and its eventual degradation through proteolysis. A range of proteins, including a-synuclein, tau, amyloid-beta and the islet amyloid polypeptide (IAPP), which are of particular interest in the context of protein deposition disorders (or pathological aggregates), are largely unstructured in solution and are often described as natively unfolded or intrinsically disordered. Interestingly, the latter pathological aggregates have been the latter intrinsically disordered systems can also be generated following proteolysis from larger proteins that are otherwise folded, such as the amyloid-β peptide (Aβ) and the amyloidogenic fragment of gelsolin. The different conformational states adopted by proteins involve a highly complex series of equilibria whose thermodynamics and kinetics in a normally functioning living system are determined by their intrinsic amino acid sequences as well as through interactions with molecular chaperones, degradation processes, and other sophisticated quality control mechanisms. Although their amino acid sequences and the biological environments in which they function have co-evolved to maintain peptides and proteins in their soluble states, in some circumstances they can convert into non-functional and potentially damaging pathological protein aggregates. The pathological species that form initially during aggregation are clusters of a relatively small number of molecules and generally retain a structural memory of the monomeric states that have generated them, thus giving rise to highly disordered, partially structured, or native-like oligomers if they originate from unfolded, partially folded, or folded monomeric states, respectively. These early aggregates are typically rather unstable, as only relatively weak intermolecular interactions are involved, and may simply dissociate to regenerate soluble species. When aggregation proceeds, however, such aggregates can undergo internal reorganization to form more stable species having β-sheet structure, a process that is often accompanied by an increase in compactness and size. These β-structured oligomers are able to grow further by self-association or through the addition of monomers, often with further and sometimes dramatic structural reorganizations, to form well-defined fibrils with cross-β structure and a high level of structural order. Such large pathological aggregates, including amyloid, amorphous, or native-like assemblies, have links with human disease as they accumulate in well-defined pathological states.

The present invention provides non-natural molecules which can not only prevent the pathological aggregation in cells but also degrade the pathological aggregates in cells.

Accordingly, the present invention provides in a first embodiment a non-natural molecule comprising the following structure with formula (A), (B) or (C):

Z₀-X₁-CP1-X₂-Z₁-M1-Z₂ (A)

Z₀-X₁-CP1-X₂-Z₁-X₃-CP2-X₄-Z₂-M1-Z₃ (B)

Z₀-X₁-CP1-X₂-Z₁-X₃-CP2-X₄-Z₂-X₅-CP3-X₆-Z₃-M1-Z₄ (C)
wherein:
Z₀ is a linker or nothing
CP1, CP2 and CP3 are identical or different capping peptides,
M1 is a moiety targeting the intracellular proteolysis system,
X₁, X₂, X₃, X₄, X₅ and X₆ are gatekeeper amino acids independently selected from 0, 1 or 2 amino acids selected from R, K, E, D or P,
in molecule (A) Z₁ is a linker and Z₂ is selected from a linker or nothing, in molecule (B) Z₁ and Z₂ are each independently a linker and Z₃ is selected from a linker or nothing, in molecule (C) Z₁, Z₂ and Z₃ are each independently a linker and Z₄ is selected from a linker or nothing.

In yet another embodiment the present invention provides in a first embodiment a non-natural molecule consisting of the following structure with formula (A), (B) or (C):

Z₀-X₁-CP1-X₂-Z₁-M1-Z₂ (A)

Z₀-X₁-CP1-X₂-Z₁-X₃-CP2-X₄-Z₂-M1-Z₃ (B)

Z₀-X₁-CP1-X₂-Z₁-X₃-CP2-X₄-Z₂-X₅-CP3-X₆-Z₃-M1-Z₄ (C)
wherein:
Z₀ is a linker or nothing
CP1, CP2 and CP3 are identical or different capping peptides,
M1 is a moiety targeting the intracellular proteolysis system,
X₁, X₂, X₃, X4, X5 and X6 are gatekeeper amino acids independently selected from 0, 1 or 2 amino acids selected from R, K, E, D or P,
in molecule (A) Z₁ is a linker and Z₂ is selected from a linker or nothing, in molecule (B) Z₁ and Z₂ are each independently a linker and Z₃ is selected from a linker or nothing, in molecule (C) Z₁, Z₂ and Z₃ are each independently a linker and Z₄ is selected from a linker or nothing.

In a preferred embodiment X₁, X₂, X₃, X₄, X₅ and X₆ are gatekeeper amino acids independently selected from 1 or 2 amino acids selected from R, K, E, or P.

In another preferred embodiment X₁, X₂, X₃, X₄, X₅ and X₆ are gatekeeper amino acids independently selected from 1 or 2 amino acids selected from R or E.

In another preferred embodiment X₁, X₂, X₃, X₄, X₅ and X₆ are gatekeeper amino acids from 1 or 2 amino acids selected from R.

In a further embodiment the invention provides a non-natural molecule comprising the following structure with formula (A), (B) or (C):

Z₀-X₁-CP1-X₂-Z₁-M1-Z₂ (A)

Z₀-X₁-CP1-X₂-Z₁-X₃-CP2-X₄-Z₂-M1-Z₃ (B)

Z₀-X₁-CP1-X₂-Z₁-X₃-CP2-X₄-Z₂-X₅-CP3-X₆-Z₃-M1-Z₄ (C)
wherein:
Z₀ is a linker or nothing
CP1, CP2 and CP3 are identical or different capping peptides, wherein the capping peptides are produced according to the method comprising the following steps:
   obtaining the 3-dimensional structure of a pathological protein aggregate or the 3-D structure of an amyloid core amino acid sequence (this amyloid core amino acid sequence corresponds with an aggregation prone region (APR) present a target protein) from a target protein that can form pathological aggregates,
   generating an in silico list of variants of said amyloid core amino acid sequence (or APR sequence) wherein each variant has 1 or 2 or 3 amino acid differences as compared to the amyloid core amino acid sequence (or APR sequence),
   calculating with a Forcefield algorithm the thermodynamic stability for every variant sequence for the interactions between i) the variant sequence and the 3D-amyloid core structure or the variant sequence and the 3-D structure of the pathological protein aggregate, this value is designated as the delta Gibbs energy of cross-interaction and ii) the variant sequence and a variant sequence seeded axial end of the APR amyloid core, this value is designated as the delta Gibbs energy of elongation,
   producing at set of candidate capping peptides wherein candidates have a negative delta G free energy for cross-interaction and a positive delta G free energy for elongation,
   experimentally testing the set of candidate capping peptides and producing one or more capping peptides.
M1 is a moiety targeting the intracellular proteolysis system,
X₁, X₂, X₃, X₄, X₅ and X₆ are gatekeeper amino acids independently selected from 0, 1 or 2 amino acids selected from R, K, E, D or P,
in molecule (A) Z₁ is a linker and Z₂ is selected from a linker or nothing, in molecule (B) Z₁ and Z₂ are each independently a linker and Z₃ is selected from a linker or nothing, in molecule (C) Z₁, Z₂ and Z₃ are each independently a linker and Z₄ is selected from a linker or nothing.

In a particular embodiment the invention provides a non-natural molecule comprising the following structure with formula (A), (B) or (C):

Z₀-X₁-CP1-X₂-Z₁-M1-Z₂ (A)

Z₀-X₁-CP1-X₂-Z₁-X₃-CP2-X₄-Z₂-M1-Z₃ (B)

Z₀-X₁-CP1-X₂-Z₁-X₃-CP2-X₄-Z₂-X₅-CP3-X₆-Z₃-M1-Z₄ (C)
wherein:
Z₀ is a linker or nothing
CP1, CP2 and CP3 are identical or different capping peptides, wherein the capping peptides are produced according to the following method comprising the following steps:
   obtaining the 3-dimensional structure of a pathological protein aggregate or the 3-dimensional structure of an amyloid core amino acid sequence (this amyloid core amino acids sequence corresponds with an aggregation prone region (APR) present a target protein) from a target protein that can form pathological aggregates,
   generating an in silico list of variants of said amyloid core amino acid sequence (or APR sequence) wherein each variant has 1 or 2 or 3 amino acid differences as compared to the amyloid core amino acid sequence (or APR sequence),
   calculating with a Forcefield algorithm the thermodynamic stability for every variant sequence for the interactions between i) the variant sequence and the 3-dimensional (3-D) amyloid core structure, or the variant sequence and the 3-D structure of the pathological protein aggregate, this value is designated as the delta Gibbs energy of cross-interaction and ii) the variant sequence and the 3-dimensional-amyloid core structure with a variant sequence interacting at its axial end or the variant sequence and the 3-dimensional structure of the pathological aggregate with a variant sequence interacting at its axial end, this value is designated as the delta Gibbs energy of elongation,
   producing at set of candidate capping peptides wherein candidates have a negative delta G free energy for cross-interaction and a positive delta G free energy for elongation,
   experimentally testing the set of candidate capping peptides and producing one or more capping peptides.
M1 is a moiety targeting the intracellular proteolysis system,
in molecule (A) Z₁ is a linker and Z₂ is selected from a linker or nothing, in molecule (B) Z₁ and Z₂ are each independently a linker and Z₃ is selected from a linker or nothing, in molecule (C) Z₁, Z₂ and Z₃ are each independently a linker and Z₄ is selected from a linker or nothing.

“A variant sequence seeded axial end of the APR amyloid core” means “the variant sequence on the 3D-structure of the APR amyloid core, which is already bound by a variant sequence”.

A capping peptide is a peptide comprising a variant of the APR sequence which APR sequence has a length of 5 to 10 amino acids and is naturally present in the amino acid sequence of a target protein which can form pathological aggregates wherein the variant of the APR sequence has one, two or three amino acid sequence differences as compared to the APR sequence, optionally said variant of the APR sequence contains at least one D-amino acid and/or at least one artificial amino acid and said capping peptide has a negative delta G free energy for cross interaction with the three-dimensional structure of the fibrils generated (or formed) from said APR sequence and a positive delta G free energy for elongation with the three-dimensional structure of the fibrils generated (or formed) from said APR sequence.

In a particular embodiment a capping peptide is a peptide comprising a variant of the APR sequence which APR sequence has a length of 5 to 10 amino acids and is naturally present in the amino acid sequence of a target protein which can form pathological aggregates wherein the variant of the APR sequence has one, two or three amino acid sequence differences as compared to the APR sequence, optionally said variant of the APR sequence contains at least one D-amino acid and/or at least one artificial amino acid and:
i) said capping peptide has a negative delta G free energy for cross interaction with the three-dimensional structure of the fibrils generated (or formed) from said APR sequence and a positive delta G free energy for elongation with the three-dimensional structure of the fibrils generated (or formed) from said APR sequence, and/or
ii) said capping peptide has a negative delta G free energy for cross interaction with the three-dimensional structure of the fibrils formed by said pathological aggregate and a positive delta G free energy for elongation with the three-dimensional structure of the fibrils formed by said pathological aggregate.

In a particular embodiment a capping peptide is a peptide consisting of a variant of the APR sequence which APR sequence has a length of 5 to 10 amino acids and is naturally present in the amino acid sequence of a target protein which can form pathological aggregates wherein the variant of the APR sequence has one, two or three amino acid sequence differences as compared to the APR sequence, optionally said variant of the APR sequence contains at least one D-amino acid and/or at least one artificial amino acid and:

i) said capping peptide has a negative delta G free energy for cross interaction with the three-dimensional structure of the fibrils generated (or formed) from said APR sequence and a positive delta G free energy for elongation with the three-dimensional structure of the fibrils generated (or formed) from said APR sequence, and/or
ii) said capping peptide has a negative delta G free energy for cross interaction with the three-dimensional structure of the fibrils formed by said pathological aggregate and a positive delta G free energy for elongation with the three-dimensional structure of the fibrils formed by said pathological aggregate.

The delta G free energy for elongation is calculated/determined by calculating/determining the interaction between a variant of the APR sequence and the three-dimensional structure of the fibrils of the APR sequence which are already bound by a variant of the APR sequence. In the alternative, the delta G free energy for elongation is calculated/determined of by calculating/determining the interaction between a variant of the APR sequence and the three-dimensional structure of the fibrils of the pathological aggregate which are already bound by a variant of the APR sequence.

The wording “fibrils formed by the APR sequence” is equivalent to the wording “amyloid core” and the amyloid core sequence is the APR sequence.

In a specific embodiment a capping peptide contains 1, or 2 or 3 D-amino acids.

In a further specific embodiment a capping peptide contains 1, 2 or 3 artificial amino acids.

In another specific embodiment a capping peptide contains 1, 2 or 3 D-amino acids and 1, 2 or 3 artificial amino acids.

The term “capping peptide” is well known in the art. A capping peptide is a polypeptide (optionally comprising non-natural amino acids or D-amino acids) which can inhibit the pathological aggregation of a target protein. Typically, capping peptides have an amino acid length of between 5 and 10 amino acids and differ by one, two or three different amino acid substitutions of a contiguous aggregation prone region (APR) naturally occurring in a target protein capable of forming pathological aggregates. Other examples of capping peptides, than the capping peptides disclosed in the examples, which can be used in the context of the present invention are described in U.S. Pat. No. 8,754,034B2 (tau aggregation inhibitors), WO2018/005866 (transthyretin inhibitors), US2019/0241613 (alpha-synuclein inhibitors), EP2719394B1 and Plumley J. A. et al (2014) J. Phys. Chem. B. 118, 3326. Other methods to generate capping peptides are known in the art. A further non-limiting example of generating capping peptides for proteins forming pathological aggregates is described in U.S. Pat. No. 8,754,034B2, on page 8, lines 31-54. Most capping peptides disclosed in the art have non-natural amino acid or D-amino acids incorporated in the structure. Remarkably the capping peptides which have been defined in the present invention already efficiently act without the need of incorporating D-amino acids or non-natural amino acids. Even more remarkably the capping peptides of the present invention have an improved action when they are designed as a combination of double (tandem capping peptides (preferably hetero-tandem capping peptides) as designated herein in the examples section, see example 11) or triple capping peptides. Tandem capping peptides can be constructed as having twice the same capping peptide sequence, as two different capping sequences. Two different capping peptide sequences (fused together in one molecule) can be directed to two different APR sequences present in the same target protein capable of forming pathological aggregates. Alternatively, two different capping peptide sequences (fused together in one molecule) can be directed to two different proteins capable of forming pathological aggregates.

In a specific embodiment the invention provides a method to produce a set of candidate capping peptides of a target protein that forms pathological aggregates comprising the following steps:
obtaining the 3-dimensional structure of the fibrils of a pathological aggregate from a target protein or obtaining the 3-D structure of an amyloid core amino acid sequence (this amyloid core amino acids sequence corresponds with an aggregation prone region (APR) naturally present a target protein) isolated from a target protein that can form pathological aggregates,
generating an in silico list of variants of said amyloid core amino acid sequence (or said APR sequence) wherein each variant has 1, or 2, or 3 amino acid differences as compared to the amyloid core amino acid sequence (or said APR sequence),
calculating with a Forcefield algorithm the thermodynamic stability for every variant sequence for the interactions between:
   i) the variant sequence and the 3-D structure of the amyloid core, or the variant sequence and the 3-D structure of the fibrils of a pathological aggregate from said protein, this value is designated as the delta Gibbs energy of cross-interaction and
   ii) the variant sequence and the 3-D structure of the amyloid core with a variant sequence interacting at its axial end or between the variant sequence and the 3-D structure of the fibrils of a pathological aggregate from said protein with a variant sequence interacting at its axial end, this value is designated as the delta Gibbs energy of elongation,
producing at set of candidate capping peptides wherein candidates have a negative delta G free energy for cross-interaction and a positive delta G free energy for elongation.

The wording “obtaining the 3-D structure of an amyloid core amino acid sequence” is equivalent with “obtaining the 3-D structure of the fibrils of the APR sequence”.

In a further specific embodiment the method comprises the experimental testing of the set of candidate capping peptides and producing one or more capping peptides.

Identification of Amyloid Forming Sequences (APR regions) in Proteins Capable of Forming Pathological Aggregates

It is well-known that one or more aggregation prone regions (APR) (also designated as aggregation causing stretches) are the cause of amyloid or non-amyloid formation of proteins which can form pathological aggregates. In other terms pathological protein aggregates comprise one or more aggregation prone regions. An aggregation prone region is herein considered as an amyloid core sequence (typically of between 5 to 10 amino acids) from which a 3-dimensional amyloid core sequence exists in the art or can be predicted by suitable algorithms such as CORDAX (see below) or can even be experimentally generated.

In the identification step beta-aggregation-prediction or aggregation prediction algorithms are used. Such algorithms are well known in the art. Typically such algorithms take into account biophysical parameters. Tango, Waltz and Zyggregator are common examples of such algorithms, but many more have been described in the art, including, but not limited to those described by Bryan et al., PLoS Comput Biol. 5(3):e1000333, 2009; Caflish, Curr Opin Chem Biol. 10(5):437-44, 2006; Conchillo-Sole et al., BMC Bioinformatics 8:65, 2007; Galzitskaya et al., PLoS Comput Biol. 29;2(12):e177, 2006; Goldschmidt et al., PNAS 107(8):3487-92, 2010; Maurer-Stroh et al., Nat Methods 7(3):237-42, 2010; Rojas Quijano et al., Biochemistry 45(14):4638-52, 2006; Saiki et al., Biochem Biophys Res Commun 343(4):1262-71, 2006; Sanchez de Groot et al., BMC Struct Biol 5:18, 2005; Tartaglia et al., Protein Sci. 14(10):2723-34, 2005; Tartaglia et al., J Mol Biol. 380(2):425-36, 2008; Thompson et al., PNAS 103(11):4074-8, 2006; Trovato et al., Protein Eng Des Sel. 20(10):521-3, 2007; Yoon and Welsh, Protein Sci. 13(8):2149-60, 2004; Zibaee et al., Protein Sci. 16(5):906-18, 2007. A particularly interesting machine learning algorithm was recently described (designated as CORDAX in Louros, N. et al (2020) Nature Communications 11:3314), which can identify APR sequences also for example in surface-exposed patches of globular proteins. Interestingly CORDAX can also predict the 3-D structure of the identified amyloid core sequences. Note that most of the algorithms are involved with the identification of amyloid aggregating sequences. Amorphous beta-aggregation also occurs and the sequence space of both forms of aggregation can overlap (Rousseau et al., Current Opinion in Structural Biology 16:118-126, 2006), and both forms of aggregation are envisaged in the application, as long as the kinetics and conditions of the reaction favour aggregation of the pathological proteins able to form pathological aggregates.

The term “pathological aggregates” refers to amyloid and non-amyloid aggregates. Amyloid aggregates can be further distinguished between amyloid fibrils and amyloid oligomers. In a preferred embodiment the pathological aggregates are intracellular aggregates. It is known that many diseases associated with pathological aggregates that are catalogued as forming extracellular pathological aggregates also form intracellular aggregates. Indeed, IAPP and amyloid-beta have intracellular and extracellular aggregates.

More examples of proteins forming pathological aggregates (particularly amyloid oligomers and amyloid fibrils) and associated pathologies, are outlined in Table 1on pages 32, 33 and 34 of Chiti F and Dobson C M (2017) Annu. Rev. Biochem. 86: 27-68). Examples of non-amyloid aggregates (or deposits which is an alternative name for aggregates) are depicted in Table 2 on pages 35 and 36 of Chiti F and Dobson C M (2017) Annu. Rev. Biochem. 86: 27-68), some non-limiting examples are TDP-43, FUS, ataxin-1 and p53.

Intracellular Protein Degradation

Intracellular protein degradation can be achieved by two main processes: proteasomal, via the Ubiquitin-Proteasome System (UPS), and (endo-)lysosomal degradation. The latter is generally divided into micro-autophagy, macro-autophagy and Chaperone-Mediated Autophagy (CMA) for intracellular substrates, and endocytosis followed by lysosome fusion for extracellular substrates. Degradation through the UPS entails recognition of misfolded species by molecular chaperones, recruitment of E3 ubiquitin ligases which label these species with ubiquitin, and in doing so, target them for degradation through the proteasome. CMA is steered by molecular chaperones, particularly Hsc70, which recognizes substrates through specific KFERQ-like motifs and translocates them to the lysosomes, where they are unfolded and transported across the lysosomal membrane through the action of LAMP2A complexes (see Glick D et al (2010) J. Pathol. 221(1): 3-12) and US20100016221). In macro-autophagy, substrates (ubiquitinated protein species, but also other cellular components) are recognized by so-called adaptor proteins such as p62, NBR1, TAX1BP1, Optn and Tollip which target them to phagophores by binding to the LC3 protein, leading to the formation of autophagosomes that ultimately fuse with lysosomes, in which autophagosome contents are degraded.

“A moiety targeting the intracellular proteolysis system” refers to a small molecule or a peptide (or peptide-like) structure which interacts with (or ‘binds to’ or ‘binds with’ which are equivalent wordings) components of the intracellular proteolysis system. In still other words “a moiety targeting the intracellular proteolysis system” is “a small molecule or a peptide which binds to (or interacts with) a protein involved in the intracellular proteolysis system”. The wording “in the intracellular proteolysis system” is equivalent with “in intracellular proteolysis”, “in intracellular degradation”, “in cellular degradation”, “in intracellular protein degradation”, “in cellular protein degradation”. Examples of proteins involved in the intracellular proteolysis system are several ubiquitin E3 ligases known in the art, or the autophagosomal protein LC3, or the autophagy adaptor protein p62, or the autophagosomal proteins TAX1BP1, NBR1, Optn and Tollip or the molecular chaperone Hsc70. This “intracellular proteolysis system” refers to the ubiquitin E3 ligase system or to the autophagy system. Over the last few decades, technologies have emerged that hijack these cellular degradation pathways for the selective degradation of proteins of interest. Proteolysis-targeting chimeric molecules (PROTACs) represent an emerging technique that currently receives much attention for therapeutic intervention.

The mechanism is based on the inhibition of protein function by hijacking a ubiquitin E3 ligase for protein degradation. PROTAC molecules consist of an E3 ligase ligand (thus binding to a ubiquitin E3 ligase protein), fused via a flexible linker to a targeting moiety (a peptide or a small molecule) for a specific protein of interest. Targeting a protein involved in the intracellular proteolysis system can be conveniently done by using a moiety (or a ligand which is equivalent wording) such as a peptide ligand or a small molecule ligand. A non-limiting list of examples is provided in Table 1. PROTACs have the potential to eliminate “undruggable” protein targets, such as transcription factors and non-enzymatic proteins, which are not limited to normal physiological substrates of the ubiquitin-proteasome system. Thus, in a specific embodiment where the non-natural molecules of the invention comprise a ubiquitin E3 ligase-targeting moiety these non-natural molecules can be considered as hetero-bifunctional PROTAC molecules. Another example of intracellular proteasomal degradation is the clean-up of intracellular proteins via the autophagy system, more particularly micro-autophagy, macro-autophagy or chaperone-mediated autophagy (CMA).

Of particular interest in the present invention are moieties targeting the autophagosomal protein LC3 via the so-called LC3 interacting regions (LIRs), or moieties targeting the autophagy adaptor protein p62 (the classical receptor for LC3-dependent autophagy), or moieties targeting the autophagy adaptor protein NBR1 (NBR1 is recruited to ubiquitin-positive protein aggregates and guides them to LC3-dependent autophagy), or moieties targeting the autophagy adaptor protein TAX1BP1 or moieties targeting the molecular chaperone Hsc70. An overview of LC3 interacting regions (LIRs), and particularly the LIR motif, is made by Birgisdottir AB et al (2013) J. of Cell Science 126, 3237-3247). In addition, there is a database available which is designated as the LIR database: http://repeat.biol.ucy.ac.cy/iLIR/, this database depicts all known LC3 interacting region sequences. These latter LC3 interacting sequences can be conveniently used in the design of the non-natural molecules of the invention wherein these sequences are moieties interacting with specific proteins involved in autophagy. Several examples of small molecules and peptides capable of interacting with proteins implicated in the autophagy cellular system are depicted in table 1 (depicted as AUTAC) and several examples of small molecules and peptides capable of interacting with E3 ubiquitin ligase proteins are depicted in Table 1 (depicted as PROTAC).

The wording “interacting with” is equivalent to “binding to”.

TABLE 1
examples of PROTAC moieties and AUTAC moieties

| Ligand ID (moiety targeting an | | |
|---|---|---|
| ubitquitin E3 ligase protein | | |
| (PROTAC) or moiety targeting a | | |
| protein involved in autophagy | Moiety (ligand) | |
| (AUTAC) | type | Reference |
| 1. PROTACs | | |
| Bestatin | Small molecule | Scheepstra, M. et al (2019) Computational |
| | | and Structural Biotechnology Journal, 17, |
| | | 160-176 |
| Compound 7 | Small molecule | Scheepstra, M. et al (2019) Computational |
| | | and Structural Biotechnology Journal, 17, |
| | | 160-176 |
| Bestatin (SNIPER) | Small molecule | Ohoka N et al (2017) J Biol Chem 292: |
| | | 4556-70 |
| MV-1 (SNIPER) | Small molecule | Ohoka N et al (2017) J Biol Chem 292: |
| | | 4556-70 |
| MZ21 | Small molecule | Zengerle M et al (2015) ACS Chem Biol |
| | | 10: 1770-7 |
| Pomalidomide | Small molecule | Scheepstra, M. et al (2019) Computational |
| | | and Structural Biotechnology Journal, 17, |
| | | 160-176 |
| Thalidomide | Small molecule | Scheepstra, M. et al (2019) Computational |
| | | and Structural Biotechnology Journal, 17, |
| | | 160-176 |
| Lenalidomide | Small molecule | Scheepstra, M. et al (2019) Computational |
| | | and Structural Biotechnology Journal, 17, |
| | | 160-176 |
| CC-122 | Small molecule | Scheepstra, M. et al (2019) Computational |
| | | and Structural Biotechnology Journal, 17, |
| | | 160-176 |
| Pomalidomide C5 | Small molecule | Scheepstra, M. et al (2019) Computational |
| | | and Structural Biotechnology Journal, 17, |
| | | 160-176 |
| Nutlin | Small molecule | Scheepstra, M. et al (2019) Computational |
| | | and Structural Biotechnology Journal, 17, |
| | | 160-176 |
| KB02 (specific for | Small molecule | Zhang X et al (2019) Nat. Chem Biol. |
| nuclear targets) | | 15(7): 737-746 |
| CCW16 | Small molecule | Carl C. Ward et al (2019) ACS Chemical |
| | | Biology 14 (11), 2430-2440 |
| Nimbolide | Small molecule | Spradlin J N et al (2019) Nat Chem Biol. |
| | | 15(7): 747-755. |
| Hydroxyprolines derivatives: | Peptide | Chu, Ting-Ting et al (2016) Cell chemical |
| ALAOYIP (SEQ ID NO: 1) | | biology. 23. 453-461 |
| Iκβα peptide: DRHDSpGLDSpM | Peptide | Sakamoto K M et al (2001) Proc Natl Acad |
| (SEQ ID NO: 2) | | Sci USA. 98(15): 8554-8559 |
| 2. AUTACs | | |
| KFERQKILDQRFFE (SEQ ID NO: 3) | Peptide | Fan, X. Et al (2014) Nat Neurosci 17, 471- |
| | | 480 |
| VKKDQGSKFERQ (SEQ ID NO: 4) | Peptide | Bauer, P. Et al (2010) Nat Biotechnol 28, |
| | | 256-263 |
| other KFERQ-like motifs (SEQ ID | Peptide | Kaushik S et al (2018) Nat Rev Mol Cell Biol. |
| NO: 5) | | 19(6): 365-381. |
| cGMP or p-fluorobenzylguanine | Small molecule | Daiki Takahashi et al (2019) Mol Cell. |
| (FBnG) | | 76(5): 797-810 |
| Javelin: NLLRLTGW (SEQ ID NO: | Peptide | Jessica B. Flechtner et al (2006) J Immunol. |
| 6) | | 177(2): 1017-1027 |
| D61 | Small molecule | Nagy, Toni A et al (2019) Antimicrobial |
| | | Agents and Chemotherapy 63. 12 - |
| | | 7 Oct. 2019. Web. |
| linking a protein of interest to be | Small molecule | Li, Zhaoyang et al (2020) Autophagy 16.1: |
| degraded to the autophagosome | | 185-87. Web |
| protein LC3 | | |
| LIR region in p62 (DDDWTHLSS, | peptide | http://repeat.biol.ucy.ac.cy/iLIR |
| SEQ ID NO: 184) | | |
| LIR region in NBR1 (SEDYIIILP, | peptide | http://repeat.biol.ucy.ac.cy/iLIR |
| SEQ ID NO: 185) | | |
| LIR region in TAX1BP1 | peptide | http://repeat.biol.ucy.ac.cy/iLIR |
| (NSDMLVVT, SEQ ID NO: 186) | | |

According to specific embodiments, the total length of the non-natural molecules described herein does not exceed 60, 55 or 50 amino acids. More particularly, the length does not exceed 40 amino acids, 30 amino acids, 25 amino acids or even 20 amino acids.

In yet another embodiment the non-natural molecules of the invention further comprise a second moiety (M2) targeting the intracellular proteolysis system and said second moiety (M2) can be fused adjacent to the M1 moiety in the structures (A), (B) or (C) shown in the different embodiments before. The fusion of the M2 moiety is preferable interrupted by a linker sequence between M1 and M2.

In yet another embodiment the non-natural molecules of the invention further comprise a second moiety (M2) targeting the intracellular proteolysis system and said second moiety (M2) is fused adjacent to the Z₀ linker in the structures (A), (B) or (C) shown in the different embodiments before.

In yet another embodiment in the non-natural molecule as described herein the capping peptides in the molecules (B) and (C) are directed to the same or different aggregation prone regions of the pathological aggregation forming target protein.

In yet another embodiment in the non-natural molecule as described herein the capping peptides in the molecules (B) and (C) are directed to aggregation prone regions of different pathological aggregating forming target proteins.

In another embodiment the present invention also includes isotopically labelled non-natural molecules, which are identical to those defined herein, but for the fact that one or more atoms are replaced by an atom having an atomic mass or mass number different from the atomic mass or mass number usually found in nature. Examples of isotopes that may be incorporated into the non-natural molecules of the present invention (e.g. in the peptide part) include isotopes of hydrogen, carbon, nitrogen, oxygen, phosphorous, sulphur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Non-natural molecules of the present invention and pharmaceutically acceptable salts of said peptides or which contain the aforementioned isotopes and/or other isotopes of other atoms are within the scope of this invention. Certain isotopically labeled non-natural molecules of the present invention, for example those into which radioactive isotopes such as ³H and ¹⁴C are incorporated, are useful in drug and/or substrate tissue distribution assays. Tritiated, i.e. ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances, isotopically labelled non-natural molecules of this invention may generally be prepared by carrying out the procedures disclosed in the Examples below, by substituting a readily available isotopically labelled reagent for a non-isotopically labelled reagent.

In yet another embodiment the non-natural molecules of the invention further comprise at least one D-alanine at the amino-terminus and/or the carboxy-terminus of said peptides.

In yet another embodiment the invention provides the non-natural molecules of the invention for use as a medicament.

In yet another embodiment the invention provides the non-natural molecules of the invention for use to treat diseases where proteins form pathological aggregates. Example 13 depicts a list of diseases caused by different pathological aggregates.

In some embodiments, the non-natural molecules of the invention may comprise one or more additional residues at the amino- and/or carboxyl-terminal ends. In some embodiments, the one or more additional residues are D-alanines. For example, a non-natural molecule may comprise one or two D-alanines at the amino- and/or carboxyl-terminal ends.

In a specific embodiment the non-natural molecules of the invention can be modified for in vivo use by the addition, at the amino- and/or carboxyl-terminal ends, of a blocking agent to facilitate survival of the relevant non-natural molecule in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by any suitable methods. For example, one or more non-naturally occurring amino acids, such as for example D-alanine, can be added to the termini. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Additionally, the peptide terminus can be modified, e.g. by acetylation of the N-terminus and/or amidation of the C-terminus. Likewise, the peptides can be covalently or noncovalently coupled to pharmaceutically acceptable “carrier” proteins prior to administration.

In another particular embodiment the non-natural molecules of the invention can contain artificial amino acids in the capping peptide part and/or in the moiety targeting the intracellular proteolysis system (when the moiety is a peptide).

Linkers

As outlined in the formulas (A), (B) and (C) above, the non-natural molecules described herein can optionally also contain linker moieties, Z₀, Z₁, Z₂, Z₃ and Z₄. According to particular embodiments, the non-natural molecules, particularly non-natural molecules consisting of peptide structures, only contain internal linkers and no N- or C-terminal linkers. Thus, in formula (A): no Z₀ and no Z₂ are present in formula (B): no Z₀ and no Z₃ are present and in formula (C): no Z₀ and no Z₄ are present. In other particular embodiments, the non-natural molecules described herein have no linker moieties Z₀, Z₁, Z₂, Z₃ and Z₄. In yet another particular embodiment, the non-natural molecules have no internal linker moieties.

The nature of the linker moieties is not vital to the invention, although long flexible linkers are typically not used. According to particular embodiments, each linker (Z) is independently selected from stretch of between 0 and 20 identical or non-identical units, wherein a unit is an amino acid, a monosaccharide, a nucleotide or a monomer. Non-identical units can be non-identical units of the same nature (e.g. different amino acids, or some copolymers). They can also be non-identical units of a different nature, e.g. a linker with amino acid and nucleotide units, or a heteropolymer (copolymer) comprising two or more different monomeric species. According to particular embodiments, the length of at least one, and particularly each Z is at least 1 unit. According to other particular embodiments, Z is 0 units. According to particular embodiments, all Z linkers are identical.

Amino acids, monosaccharides and nucleotides and monomers have the same meaning as in the art. Note that particular examples of monomers include mimetics of natural monomers, e.g. non-proteinogenic or non-naturally occurring amino acids (e.g. carnitine, GABA, and L-DOPA, hydroxyproline and selenomethionine), peptide nucleic acid monomers, and the like. Examples of other suitable monomers include, but are not limited to, ethylene oxide, vinyl chloride, isoprene, lactic acid, olefins such as ethylene, propylene, amides occurring in polymers (e.g. acrylamide), acrylonitrile-butadiene-styrene monomers, ethylene vinyl acetate, and other organic molecules that are capable of polymer formation.

According to alternative embodiments, the linker units are chemical linkers, such as those generated by carbodiimide coupling. Examples of suitable carbodiimides include, but are not limited to, 1-Ethyl-3-(3-dimethylaminopropyl)carbodiimide (EDC), N,N′-Diisopropylcarbodiimide (DIC), and Dicyclohexylcarbodiimide (DCC). Another particularly envisaged chemical linker is 4, 7, 10-trioxatridecan-succinic acid (sometimes also designated as 4, 7, 10-trioxatridecan-succinamic acid) or Ttds.

According to specific embodiments, at least one, and particularly all, Z linkers are of between 0 and 10 units of the same nature, particularly between 0 and 5 units of the same nature. If the linkers are flexible, it is particularly envisaged to use short linkers. The use of short linkers prevents that two CP- stretches of the same non-natural molecule will fold back on itself, as this would make them less accessible to target the amyloid proteins of interest. Also, by making sure the different CP-stretches of one molecule cannot interact with each other, solubility of the molecule is increased. According to particular embodiments, the linkers are so short that they do not allow the folding of the CP-stretches in antiparallel fashion. For instance, for amino acids, at least three or four amino acids are required to make a complete turn, so linkers of no more than four or of no more than three amino acids are particularly envisaged. This also depends on the nature of the amino acids, so the use of amino acids that do not have a particular structural propensity, or a propensity for a kinked structure, such as G, S and P is particularly envisaged. According to particular embodiments, at least one Z moiety, is a peptide or polypeptide linker. Particularly envisaged sequences of such linkers include, but are not limited to, PPP, PP or GS. For Z moieties that are made up of amino acids, one can take into account the primary structure (e.g. in the sequence of the linker include many amino acids without a penchant for a particular structure), but also the secondary or tertiary structure. For instance, one can choose amino acids that form no particular secondary structure, or form a (linear) alpha helix. Or, amino acids can be chosen so that they do not form a stable tertiary structure, as this might result in the CP moieties becoming inaccessible. The amino acid linkers may form a random coil. Another particularly envisaged linker is polyethylene glycol (PEG), i.e. an oligomer or polymer of monomeric ethylene oxide groups. PEG oligomers are often abbreviated whilst indicating the number of monomeric units, e.g. PEG₂, PEG₃ or (PEG)4. According to particular embodiments, at least one Z moiety is a PEG oligomer (PEG in short). According to further particular embodiments, all Z moieties are PEG moieties.

One particular example where longer linkers are favoured over other linkers are those instances where at least two different amyloid proteins, in the same organism, are targeted (i.e. the CP1 and CP2 and/or CP3 moieties correspond to aggregation-inducing regions of more than one amyloid forming protein). To ensure that the molecule can (e.g. simultaneously) interact with more than one amyloid forming protein, it may be beneficial to increase the distance between the different targeting CP moieties, so that the interaction is not prevented due to steric hindrance. In these instances, the Z linker may be a stretch of between 0 and 100 identical or non-identical units, wherein a unit is an amino acid, a monosaccharide, a nucleotide or a monomer; or of between 0 and 90, 0 and 80, 0 and 70, 0 and 60, 0 and 50, 0 and 40, 0 and 30 or 0 and 20. Particularly, the minimal length of the Z linker is at least 1 unit, at least 2 units, at least 3 units, at least 4 units, or at least 5 units.

When longer linkers are used, they are preferably not identical to sequences of the proteins from which the at least one CP region is derived. According to very particular embodiments, a linker of more than 20 units is not a peptidic linker, i.e. the units are not amino acids. According to alternative embodiments, longer linkers can be peptidic linkers, but peptidic linkers containing repeat motifs (e.g. GS, GGS, PP linkers or other linkers containing mono-, di- or tri- amino acid repeats). Particularly, the linker is essentially free of secondary polypeptide structure, for example of stretches of alpha-helix or beta-sheet. Any predisposition of the polypeptide linker toward a motif of polypeptide secondary structure will necessarily limit the degree of spatial freedom enjoyed by the linker's ends.

Other Moieties

The non-natural molecule can further comprise (or can be further fused to or coupled to) still other moieties. For all moieties, the nature of the fusion or linker is not vital to the invention, as long as the moiety and the non-natural molecule can exert their specific function. According to particular embodiments, the moieties which are fused to the non-natural molecules can be cleaved off (e.g. by using a linker moiety that has a protease recognition site). This way, the function of the moiety and the molecule can be separated, which may be particularly interesting for larger moieties, or for embodiments where the moiety is no longer necessary after a specific point in time (e.g. a tag that is cleaved off after a separation step using the tag).

It is particularly envisaged that the molecule further comprises a detectable label. The detectable label can be N- or C-terminally or even internally fused to the molecule (e.g. through the linker, or the linker can be used as the detectable label). Alternatively, the detectable label can refer to the use of one or more labeled amino acids in one or more of the CP-M-X-Z moieties of the molecule (e.g. fluorescently or radioactively labeled amino acids).

Note that, for embodiments where Z₀ is present in the non-natural molecules (or in formula (A) wherein Z₂ is present or in formula (B) wherein Z₃ is present or in formula (C) wherein Z₄ is present, the detectable label can be fused to the Z₀ (or Z₂ or Z₃ or Z₄) linker moiety. The linkers used to add the tag to the molecules may be both long and flexible. However, the actual way in which the detectable label is attached to the molecules is not vital to the invention and will typically depend on the nature of the label used and/or the purpose of labeling (which may determine the required proximity). Note that in principle any known label for molecules of proteinaceous nature can be used, as long as the label can be detected. Particularly envisaged labels include, but are not limited to, tags, fluorescent labels, enzyme substrates, enzymes, quantum dots, nanoparticles which may be (para)magnetic, radiolabels, optical labels and the like.

As with other moieties, since the molecules have two ends, it is envisaged that the molecules will be fused to another moiety (e.g. a label) at both its N- and C-terminus. These two labels can be identical (yielding a stronger signal) or different (for different detection purposes). Moieties such as labels can be fused through Z₀ and/or Z₂ or Z₃ or Z₄ linkers, or through longer linkers.

According to particular embodiments, the detectable label is not GFP or biotin. According to other particular embodiments, biotin or GFP can be the detectable label.

According to other particular embodiments, the non-natural molecules may be fused to other moieties, e.g. to extend their half-life in vivo. Apart from increasing stability, such moieties may also increase solubility of the molecule they are fused to. Although the presence of gatekeepers (the numbered X₁-X₂-X₃-X₄ moieties in the respective formulas (A), (B) and (C) is in principle helpful to prevent keep the non-natural molecules in solution, in many cases we have found that the X-moieties can be omitted. In particular embodiments the further addition of a moiety that increases solubility may provide easier handling of the molecules, and particularly improve stability and shelf-life. A well-known example of such moiety is PEG (polyethylene glycol). This moiety is particularly envisaged, as it can be used as linker as well as solubilizing moiety. Other examples include peptides and proteins or protein domains, or even whole proteins (e.g. GFP). In this regard, it should be noted that, like PEG, one moiety can have different functions or effects. For instance, a flag tag (sequence DYKDDDDK) is a peptide moiety that can be used as a label, but due to its charge density, it will also enhance solubilisation. PEGylation has already often been demonstrated to increase solubility of biopharmaceuticals (e.g. Veronese and Mero, BioDrugs. 2008; 22(5):315-29). Adding a peptide, polypeptide, protein or protein domain tag to a molecule of interest has been extensively described in the art. Examples include, but are not limited to, peptides derived from synuclein (e.g. Park et al., Protein Eng. Des. Sel. 2004; 17:251-260), SET (solubility enhancing tag, Zhang et al., Protein Expr Purif 2004; 36:207-216), thioredoxin (TRX), Glutathione-S-transferase (GST), Maltose-binding protein (MBP), N-Utilization substance (NusA), small ubiquitin-like modifier (SUMO), ubiquitin (Ub), disulfide bond C (DsbC), Seventeen kilodalton protein (Skp), Phage T7 protein kinase fragment (T7PK), Protein G B1 domain, Protein A IgG ZZ repeat domain, and bacterial immunoglobulin binding domains (Hutt et al., J Biol Chem.; 287(7):4462-9, 2012). The nature of the tag will depend on the application, as can be determined by the skilled person.

Apart from extending half-life, non-natural molecules may be fused to moieties that alter other or additional pharmacokinetic and pharmacodynamic properties. For instance, it is known that fusion with albumin (e.g. human serum albumin), albumin-binding domain or a synthetic albumin-binding peptide improves pharmacokinetics and pharmacodynamics of different therapeutic proteins (Langenheim and Chen, Endocrinol.; 203(3):375-87, 2009). Another moiety that is often used is a fragment crystallizable region (Fc) of an antibody. The nature of these moieties is not vital to the invention and can be determined by the person skilled in the art depending on the application.

Other moieties which are also envisaged in combination with the non-natural molecules described herein are targeting moieties. For instance, the molecules may be fused to e.g. an antibody, a peptide or a small molecule with a specificity for a given target, and the non-natural molecule inhibits amyloid formation in the cell comprising the target amyloid protein because the targeting moiety present in the non-natural molecule guides the non-natural molecule towards the cell comprising the amyloid protein.

Note however that targeting moieties are not necessary, as the molecules themselves are able to find their target through specific sequence recognition. Thus, according to alternative embodiments, the molecules can effectively be used as targeting moiety and be further fused to other moieties such as drugs or small molecules. By targeting the molecules to specific proteins (e.g. proteins only occurring in a particular cell type or cell compartment), these compounds can be targeted to the specific cell type/compartment. Thus, for instance, drugs can selectively be delivered to cells comprising the amyloid target protein.

According to yet other embodiments, the molecules can further comprise a sequence which mediates cell penetration (or cell translocation), i.e. the molecules are further modified through the recombinant or synthetic attachment of a cell penetration sequence. Note that however the non-natural molecules of the invention efficiently enter the cells without a cell penetration sequence. The non-natural molecule (e.g. as a polypeptide) may be further fused or chemically coupled to a sequence facilitating transduction of the fusion or chemical coupled proteins into eukaryotic cells. Cell-penetrating peptides (CPP) or protein transduction domain (PTD) sequences are well known in the art and include, but are not limited to the HIV TAT protein, a polyarginine sequence, penetratin and pep-1. Still other commonly used cell-permeable peptides (both natural and artificial peptides) are disclosed e.g. in Sawant and Torchilin, Mol Biosyst. 6(4):628-40, 2010; Noguchi et al., Cell Transplant. 19(6):649-54, 2010 and Lindgren and Langel, Methods Mol Biol. 683:3-19, 2011.

Typical for CPP is their charge, so it is possible that some charged molecules described herein do not need a CPP to enter a cell. Indeed, as will be shown in the examples, it is possible to target signal peptides or intracellular regions, which require that the molecules are taken up by the cell, and this happens without fusion to a CPP.

In those instances where other moieties are fused to the molecules, it is envisaged in particular embodiments that these moieties can be removed from the molecule. Typically, this will be done through incorporating a specific protease cleavage site or an equivalent approach. This is particularly the case where the moiety is a large protein: in such cases, the moiety may be cleaved off prior to using the molecule in any of the methods described herein (e.g. during purification of the molecules). The cleavage site may be incorporated separately or may be an integral part of the external Z linker (for example is the moiety is N-terminal or C-terminal from the non-natural molecule). According to very specific embodiments, the moiety may be part of an internal Z linker, or may even be the whole Z linker. By way of example, a molecule with CP=2 could have the following structure: X₁-CP1-X₂-Z₁-X₃-CP2-X₄-M, wherein Z₁ (in part or in whole) is a hexahistidine sequence: this is then both the linker and detection sequence. Although it is possible, in those instances normally no cleavage site will be built in, as this would lead to cleaving of the molecule itself. Note that according to the embodiments where the additional moiety is fused internally, only non-proteinaceous (e.g. PEG) or peptide sequences with limited length (less than 30, 20 or 10 amino acids, cf. above) are envisaged as solubilization moieties.

Otherwise, the protein domain might interfere with the prevention of amyloid aggregation.

According to specific embodiments, the total length of the non-natural molecules described herein does not exceed 50 amino acids. More particularly, the length does not exceed 40 amino acids, 30 amino acids, 25 amino acids or even 20 amino acids. According to further specific embodiments where the molecules are fused to further moieties, the length limitation only applies to the X₁-CP1-X₂-Z₁-X₃-CP2-X₄-M1 or the X₁-CP1-X₂-Z₁-X₃-CP2-X₄-Z₂-CP3-X₅-Z₃-M1 part of the total molecule (and thus not e.g. to the label). Thus, if a cleavage site has been built in the molecule, the length restriction typically applies to the length after cleavage.

Gatekeeper Amino Acids

In the non-natural molecules described herein, the capping peptide sequences are optionally flanked on both sides by 0, 1 or 2 specific amino acids (the X₁ and X₂ moieties in formula (A), the X₁, X₂, X₃ and X₄ moieties in formula (B) and the X₁, X₂, X₃, X₄, X₅ and X₆ moieties in formula (C)) that have low beta-aggregation potential. These are sometimes referred to as gatekeeper residues (Pedersen et al., J Mol Biol 341: 575-588, 2004), and are optional in keeping the capping peptides in the non-natural molecules from self-aggregation. Gatekeeper residues can be used in the non-natural molecules of the invention to increase the solubility. In these instances preferred gatekeeper residues are positively charged gatekeeper residues such as R and E. However, it should be said that in another preferred embodiment no gatekeeper amino acids are flanking the capping peptide sequences in the non-natural molecules.

In the native state of proteins, aggregation is often contained or opposed by naturally occurring charged residues but also e.g. prolines and glycines at the flanks of aggregating sequence segments. These effectively act as gatekeeper residues, i.e. residues that do not necessarily stabilize the native state, but which block the formation of unwanted misfolded or aggregated states by, for example, steric or electrostatic clashes.

These gatekeeper residues are particularly selected from R, K, E, D, P, N, S, H, G and Q residues. Even more particularly gatekeeper residues are selected from R, K, P, D or E. Even more particularly gatekeeper residues are selected from R and E.

Recombinant Production of Non-Natural Molecules

In a particular embodiment where the non-natural molecule entirely consists of natural amino acids the recombinant production of the specific non-natural molecule may be envisaged. However, care has to be taken that the moiety which interacts with the intracellular proteolysis system is not recognized by the host cell used for recombinant expression. Therefore, in preferred aspects the non-natural molecule to be expressed by the host cell is exogenous to the host cell and does not interact with the intracellular proteolysis system of the recombinant host cell. For example, heterologous expression of the non-natural molecules in prokaryotic hosts, such as E. coli, will generally not cause any problem to express. Recombinant vectors are ideally suited as a vehicle to carry the nucleic acid sequence of interest inside a cell where the protein to be downregulated is expressed, and drive expression of the nucleic acid in said cell. The recombinant vector may persist as a separate entity in the cell (e.g. as a plasmid or a viral or non-viral carrier), or may be integrated into the genome of the cell.

Accordingly, suitable recombinant cells are provided herein comprising a nucleic acid molecule encoding (or with a sequence encoding) a molecule as herein described or comprising a recombinant vector that contains a nucleic acid molecule encoding such non-natural molecule molecule. The cell may be a prokaryotic or eukaryotic cell. In the latter case, it may be preferably a yeast, algae or plant or even an animal cell (e.g. insect, mammal or human cell). According to particular embodiments, the cell is provided as a cell line.

A recombinant non-natural molecule may be manufactured using suitable expression systems comprising bacterial cells, yeast cells, animal cells, insect cells, plant cells or transgenic animals or plants.

The recombinant non-natural molecule may be purified by any conventional protein or peptide purification procedure close to homogeneity and/or be mixed with additives. In yet another embodiment said non-natural molecule is expressed, purified and further modified into a chemically modified polypeptide. Chemical synthesis enables the conjugation of other small molecules or incorporation of non-natural amino acids by design. Incorporation of non-natural amino acids into the peptide opens up the possibility for greater chemical diversity, analogous to small-molecule medicinal chemistry approaches for developing high-affinity, high-specificity molecular recognition. Non-natural amino acids can also prevent rapid degradation of the peptide non-natural molecule by rendering the peptide unrecognizable to proteases (e.g. serum or stomach). In yet another embodiment the non-natural molecules of the invention comprise modified amino acids such as a D-amino acid or a chemically modified amino acid. In yet another embodiment said non-natural molecule consists of a mixture of natural amino acids and unnatural amino acids. In yet another embodiment the half-life of a non-natural molecule can be extended by modifications such as glycosylation (Haubner R. et al (2001) J. Nucl. Med. 42, 326-336), conjugation with polyethylene glycol (PEGylation, see Kim TH et al (2002) Biomaterials 23, 2311-2317), or engineering the peptide to associate with serum albumin (see Koehler M F et al (2002) Bioorg. Med. Chem. Lett. 12, 2883-2886).

Viral Transduction/Gene Therapy

In a particular embodiment when the non-natural molecule consists entirely of (natural) amino acids these non-natural molecules can be administered as transgenes (i.e. as nucleic acids encoding the molecules), it goes without saying that the non-natural molecules according to these embodiments are entirely of polypeptide nature, since they need to be able to be encoded. I.e., all numbered Z, CP, (optionally X), and M moieties present in the non-natural molecules are of polypeptide nature. Medical applications in which transgenic delivery is envisaged include, but are not limited to, gene therapy methods (e.g. using lentiviruses) or stem cell applications.

In a particular embodiment the administration of the non-natural molecules of the invention, when peptidic in nature, can be carried out by gene therapeutic methods. Thus, non-natural molecules of the invention (when these non-natural molecules consist purely of amino acids) can be encoded by nucleic acids and such a nucleic acid is provided in a vector. It is particularly envisaged that such a non-natural molecule can be administered through gene therapy. ‘Gene therapy’ as used herein refers to therapy performed by the administration to a subject of an expressed or expressible nucleic acid. For such applications, the nucleic acid molecule or vector as described herein allow for production of the non-natural molecule within a cell. A large number of methods for gene therapy are available in the art and include, for instance (adeno-associated) virus mediated gene silencing, or virus mediated gene therapy (e.g. US 20040023390; Mendell et al 2017, N Eng J Med 377:1713-1722). A plethora of delivery methods are well known to those of skill in the art and include but are not limited to viral delivery systems, microinjection of DNA plasmids, biolistics of naked nucleic acids, use of a liposome. In vivo delivery by administration to an individual patient occurs typically by systemic administration (e.g., intravenous, intraperitoneal infusion or brain injection; e.g. Mendell et al 2017, N Eng J Med 377:1713-1722). Where said non-natural molecules are provided as a nucleic acid or a vector, it is more particularly also envisaged that such non-natural molecules are administered through delivery methods and vehicles that comprise nanoparticles or lipid-based delivery systems such as artificial exosomes, which may also be cell-specific, and suitable for delivery of non-natural molecules.

Synthesis of Non-Natural Molecules

In specific embodiments the non-natural molecules (or at least the peptidic part thereof) of the invention can be produced according to several peptide synthesis methods known in the art. The peptide synthesis method may be any of, for example, a solid phase synthesis process and a liquid phase synthesis process. That is, the object non-natural molecules can be produced by repeating condensation of a partial peptide or amino acid capable of constituting compound (I) and the remaining portion (which may be constituted by two or more amino acids) according to a desired sequence. When a product having the desirable sequence has a protecting group, the object peptide can be produced by eliminating a protecting group. Examples of the condensing method and eliminating method of a protecting group to be known include methods described in the following (1)-(5). (1) M. Bodanszky and M. A. Ondetti: Peptide Synthesis, Interscience Publishers, New York (1966), (2) Schroeder and Luebke: The Peptide, Academic Press, New

York (1965), (3) Nobuo lzumiya, et al.: Peptide Gosei-no-Kiso to Jikken (Basics and experiments of peptide synthesis), published by Maruzen Co. (1975), (4) Haruaki Yajima and Shunpei Sakakibara: Seikagaku Jikken Koza (Biochemical Ex-periment) 1, Tanpakushitsu no Kagaku (Chemistry of Proteins) IV, 205 (1977) and (5) Haruaki Yajima, ed.: Zoku lyakuhin no Kaihatsu (A sequel to Development of Pharmaceuticals), Vol. 14, Peptide Synthesis, published by Hirokawa Shoten. After the reaction, the peptides can be purified and isolated using conventional methods of purification, such as solvent extraction, distillation, column chromatography, liquid chromatography, recrystallization, etc., in combination thereof. When the peptide obtained by the above-mentioned method is in a free form, it can be converted to a suitable salt by a known method; conversely, when the peptide is obtained in the form of a salt, the salt can be converted to a free form or other salt by a known method. The starting compound may also be a salt. Examples of such salt include those exemplified as salts of the peptides mentioned bellow. For condensation of protected amino acid or peptide, various activation reagents usable for peptide synthesis can be used, which are particularly preferably trisphosphonium salts, tetramethyluronium salts, carbodiimides and the like. Examples of the trisphosphonium salt include benzotriazol yloxytris(pyrrolizino)phosphoniumhexafluorophosphate (PyBOP), bromotris(pyrrolizino)phosphoniumhexafluorophosphate (PyBroP), 7-azabenzotriazol yloxytris(pyrrolizino)phosphoniumhexafluorophosphate (PyAOP), examples of the tetramethyluronium salt include 2-(1H-benzotriazol-1-yl)-1,1,3,3-hexafluorophosphate (HBTU), 2-(7-azabenzotriazol-1-yl)-1,1,3,3-hexafluorophosphate (HATU), 2-(1H-benzotriazol-1-yl)-1,1,3,3-tetramethyluroniumtetrafluoroborate (TBTU), 2-(5-norbornane-2,3-dicarboxyimide)-1,1,3,3-tetramethyluroniumtet-rafluoroborate (TNTU), O-(N-succimidyl)-1,1,3,3-tetramethyluroniumtetrafluoroborate (TSTU), and examples of the carbodiimide include DCC, N,N′-diisopropylcarbodiimide (DIPCDI), N-ethyl-N′-(3-dimethylaminopropyl)carbodiimide hydrochloride (EDCl.HCl) and the like. For condensation using these, addition of a racemization inhibitor (e.g., HONB, HOBt, HOAt, HOOBt etc.) can be used. A solvent to be used for the condensation can be appropriately selected from those known to be usable for peptide condensation reaction. For example, acid amides such as anhydrous or water-containing N,N-dimethylformamide, N,N-dimethylacetamide, N-methylpyrrolidone and the like, halogenated hydrocarbons such as methylene chloride, chloroform and the like, alcohols such as trifluoroethanol, phenol and the like, sulfoxides such as dimethyl-sulfoxide and the like, tertiary amines such as pyridine and the like, ethers such as dioxane, tetrahydrofuran and the like, nitriles such as acetonitrile, propionitrile and the like, esters such as methyl acetate, ethyl acetate and the like, an appropriate mixture of these and the like can be used. Reaction temperature is appropriately selected from the range known to be usable for peptide binding reactions, and is normally selected from the range of about −20 C (“C” represents “degrees Celsius”) to 50 degrees C. An activated amino acid derivative is normally used from 1.5 to 6 times in excess. In phase synthesis, when a test using the ninhydrin reaction reveals that the condensation is insufficient, sufficient condensation can be conducted by repeating the condensation reaction without elimination of protecting groups. If the condensation is yet insufficient even after repeating the reaction, unreacted amino acids can be acylated with acetic anhydride, acetylimidazole or the like so that an influence on the subsequent reactions can be avoided. Examples of the protecting groups for the amino groups of the starting amino acid include Z, Boc, tert-pentyloxycarbonyl, isobornyloxycarbonyl, 4-methoxybenzyloxycarbonyl, Cl—Z, Br—Z, adamantyloxycarbonyl, trifluoroacetyl, phthaloyl, formyl, 2-nitrophenylsulphenyl, diphenylphosphinothioyl, Fmoc, trityl and the like. Examples of the carboxyl-protecting group for the starting amino acid include allyl, 2-adamantyl, 4-nitrobenzyl, 4-methoxybenzyl, 4-chlorobenzyl, phenacyl and benzy-loxycarbonylhydrazide, tert-butoxycarbonylhydrazide, tritylhydrazide and the like, in addition to the above-mentioned C₁₋₆ alkyl group, C₃₋₁₀ cycloalkyl group, C₇₋₁₄ aralkyl group. The hydroxyl group of serine or threonine can be protected, for example, by esterification or etherification. Examples of the group suitable for the esterification include lower (C₂₋₄) alkanoyl groups such as an acetyl group and the like, aroyl groups such as a benzoyl group and the like, and the like, and a group derived from an organic acid and the like. In addition, examples of the group suitable for etherification include benzyl, tetrahydropyranyl, tert-butyl(Bu.sup.t), trityl (Trt) and the like. Examples of the protecting group for the phenolic hydroxyl group of tyrosine include Bzl, 2,6-dichlorobenzyl, 2-nitrobenzyl, Br--Z, tert-butyl and the like. Examples of the protecting group for the imidazole of histidine include Tos, 4-methoxy-2,3,6-trimethylbenzenesulfonyl (Mtr), DNP, Bom, Bum, Boc, Trt, Fmoc and the like.

Examples of the protecting group for the guanidino group of arginine include Tos, Z, 4-methoxy-2,3,6-trimethylbenzenesulfonyl (Mtr), p-methoxybenzenesulfonyl (MBS), 2,2,5,7,8-pentamethylchromane-6-sulfonyl (Pmc), mesitylene-2-sulfonyl (Mts), 2,2,4,6,7-pentamethyldihydrobenzofuran-5-sulfonyl (Pbf), Boc, Z, NO₂ and the like. Examples of the protecting group for a side chain amino group of lysine include Z, Cl—Z, trifluoroacetyl, Boc, Fmoc, Trt, Mtr, 4,4-dimethyl-2,6-dioxocyclohexylideneyl (Dde) and the like.

Examples of the protecting group for indolyl of tryptophan include formyl (For), Z, Boc, Mts, Mtr and the like. Examples of the protecting group for asparagine and glutamine include Trt, xanthyl (Xan), 4,4′-dimethoxybenzhydryl (Mbh), 2,4,6-trimethoxybenzyl (Tmob) and the like. Examples of activated carboxyl groups in the starting material include corresponding acid anhydride, azide, active esters [ester with alcohol (e.g., pentachlorophenol, 2,4,5-trichlorophenol, 2,4-dinitrophenol, cyanomethylalcohol, paranitrophenol, HONB, N-hydroxysuccimide, 1-hydroxybenzotriazole (HOBt), 1-hydroxy-7-azabenzotriazole(HOAt))] and the like. Examples of the activated amino group in the starting material include corresponding phosphorous amide. Examples of the method for removing (eliminating) a protecting group include a catalytic reduction in a hydrogen stream in the presence of a catalyst such as Pd-black or Pd-carbon; an acid treatment using anhydrous hydrogen fluoride, methanesulfonic acid, trifluoromethanesulfonic acid, trifluoroacetate, trimethylsilyl bromide (TMSBr), trimethylsilyl trifluoromethanesulfonate, tetrafluoroboric acid, tris(trifluoro)boric acid, boron tribromide, or a mixture solution thereof; a base treatment using diisopropy-lethylamine, triethylamine, piperidine, piperazine or the like; and reduction with sodium in liquid ammonia, and the like. The elimination reaction by the above-described acid treatment is generally carried out at a temperature of -20 C to 40 C; the acid treatment is efficiently conducted by adding a cation scavenger such as anisole, phenol, thioanisole, metacresol and paracresol; dimethylsulfide, 1,4-butanedithiol, 1,2-ethanedithiol and the like. Also, a 2,4-dinitrophenyl group used as a protecting group of the imidazole of histidine is removed by thiophenol treatment; a formyl group used as a protecting group of the indole of tryptophan is removed by deprotection by acid treatment in the presence of 1,2-ethanedithiol, 1,4-butanedithiol, or the like, as well as by alkali treatment with dilute sodium hydroxide, dilute ammonia, or the like.

In addition, the non-natural molecules of the invention may be a solvate (e.g., hydrate) or a non-solvate (e.g. non-hydrate). The non-natural molecules may be labeled with an isotope (e.g. ³, ¹⁴C, ³⁵S, ¹²⁵I) or the like. Furthermore, the non-natural molecules may be a deuterium conversion form wherein ¹H is converted to ²H(D). Peptides labeled or substituted with an isotope can be used as, for example, a tracer (PET tracer) for use in Positron Emission Tomography (PET) and is useful in the fields of medical diagnosis and the like.

For the non-natural molecules (when purely consist of peptides) mentioned herein, the left end is the N-terminal (amino terminal) and the right end is the C-terminal (carboxyl terminal) in accordance with the conventional peptide marking. The C-terminal of peptide may be any of an amide (—CONH₂), a carboxyl group (—COOH), a carboxylate (—COO⁻), an alkylamide (—CONHR), and an ester (—COOR). Particularly, amide (—CONH₂) is preferable. The non-natural molecules may be in a salt form. Examples of such salt include metal salts, ammonium salts, salts with organic base, salts with inorganic acid, salts with organic acid, salts with basic or acidic amino acid, and the like.

In certain embodiments, the non-natural molecules may also be in a prodrug form. A prodrug means a compound which is converted to a functional peptide of the invention with a reaction due to an enzyme, gastric acid, etc. under the physiological condition in the living body, that is, a compound which is converted to a peptide of the invention with oxidation, reduction, hydrolysis, etc. according to an enzyme; a compound which is converted to a non-natural molecule of the invention by hydrolysis etc. due to gastric acid, etc. Examples of a prodrug of a peptide of the invention include a compound wherein an amino of the peptide is acylated, alkylated or phosphorylated (e.g., compound wherein amino of the peptide is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like); a compound wherein a hydroxy of the peptide is acylated, alkylated, phosphorylated or borated (e.g., a compound wherein a hydroxy of the peptide is acetylated, palmytoylated, propanoylated, pivaloylated, succinylated, fumarylated, alanylated or dimethylaminomethylcarbonylated); a compound wherein a carboxy of the peptide is esterified or amidated (e.g., a compound wherein a carboxy of the peptide is C₁₋₆ alkyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycar-bonylethyl esterified or methylamidated) and the like. Among others, a compound wherein carboxy of compound (I) is esterified with C₁₋₆ alkyl such as methyl, ethyl, tert-butyl or the like is preferably used. These compounds can be produced from a peptide by a method known per se. A prodrug of a peptide of the invention may also be one which is converted into a peptide of the invention under a physiological condition, such as those described in IYAKUHIN no KAIHATSU (Development of Pharmaceuticals), Vol. 7, Design of Molecules, p. 163-198, Published by HIROKAWA SHOTEN (1990). In the present specification, the prodrug may form a salt. Examples of such a salt include those exemplified as the salt of a peptide of the invention. A peptide of the invention may form a crystal.

Crystals having a singular crystal form or a mixture of plural crystal forms are also included in a peptide of the invention. Crystals can be produced by crystallizing a peptide of the invention according to a crystallization method known per se. In addition, a peptide of the invention may be a pharmaceutically acceptable co-crystal or co-crystal salt. Here, the co-crystal or co-crystal salt means a crystalline substance consisting of two or more particular substances which are solids at room temperature, each having different physical properties (e.g. structure, melting point, heat of melting, hygroscopicity, solubility, stability etc.). The cocrystal and cocrystal salt can be produced by co-crystallization known per se. The crystal of a peptide of the invention is superior in physicochemical properties (e.g., melting point, solubility, stability) and biological properties (e.g. pharmacokinetics (absorption, distribution, metabolism, excretion), efficacy expression), and thus it is extremely useful as a medicament.

Administration of the Non-Natural Molecules of the Invention—Pharmaceutical Compositions Comprising the Non-Natural Molecule of the Invention

In one embodiment, the non-natural molecules of the invention are administered directly to a subject. Generally, the compounds of the invention will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, intracerebrally or intrapulmonarily. In another embodiment, the intracerebral or intratracheal delivery can be accomplished using a dosage pump. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Wide variations in the needed dosage are to be expected in view of the variety of non-natural molecules and variants possible and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-or more fold).

In certain embodiments, the non-natural molecules of the invention comprise at least one modified terminus, e.g. to protect the non-natural molecule against degradation. In some embodiments, the N-terminus is acetylated and/or the C-terminus is amidated. In certain embodiments, the non-natural molecules of the invention comprise at least one non-natural amino acid (e.g., 1, 2, 3, or more) or at least one terminal modification (e.g. 1 or 2). In some embodiments, the non-natural molecule comprises at least one non-natural amino acid and at least one terminal modification.

The non-natural molecules of the present invention can optionally be delivered in conjunction with other therapeutic agents. The additional therapeutic agents can be delivered concurrently with the non-natural molecules of the invention. As used herein, the word “concurrently” means sufficiently close in time to produce a combined effect (that is, concurrently can be simultaneously, or it can be two or more events occurring within a short time period before or after each other). The kit may further comprise additional reagents for carrying out the methods (e.g., buffers, containers, additional therapeutic agents) as well as instructions. As a further aspect, the invention provides pharmaceutical formulations and methods of administering the same to achieve any of the therapeutic effects discussed above. The pharmaceutical formulation may comprise any of the reagents discussed above in a pharmaceutically acceptable carrier, e.g. a non-naturally occurring peptide or variant thereof. By “pharmaceutically acceptable” it is meant a material that is not biologically or otherwise undesirable, i.e., the material can be administered to a subject without causing any undesirable biological effects such as toxicity. The formulations of the invention can optionally comprise medicinal agents, pharmaceutical agents, carriers, adjuvants, dispersing agents, diluents, and the like. The non-natural molecules of the invention can be formulated for administration in a pharmaceutical carrier in accordance with known techniques. See, e.g. Remington, The Science And Practice of Pharmacy (Ed. 2014). In the manufacture of a pharmaceutical formulation according to the invention, the non-natural molecule (including the physiologically acceptable salts thereof) is typically admixed with, inter alia, an acceptable carrier. The carrier can be a solid or a liquid, or both, and is preferably formulated with the peptide as a unit-dose formulation, for example, a tablet, which can contain from 0.01 or 0.5% to 95% or 99% by weight of the peptide. One or more non-natural molecules can be incorporated in the formulations of the invention, which can be prepared by any of the well-known techniques of pharmacy. A further aspect of the invention is a method of treating subjects in vivo, comprising administering to a subject a pharmaceutical composition comprising a non-natural molecule of the invention in a pharmaceutically acceptable carrier, wherein the pharmaceutical composition is administered in a therapeutically effective amount. Administration of the non-natural molecules of the present invention to a human subject or an animal in need thereof can be by any means known in the art for administering compounds. The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, intracerebral and inhalation administration, administration to the liver by intraportal delivery, as well as direct organ injection (e.g., into the liver, into the brain for delivery to the central nervous system, into the pancreas, or into a tumor or the tissue surrounding a tumor). The most suitable route in any given case will depend on the nature and severity of the condition being treated and on the nature of the particular non-natural molecule which is being used.

For injection, the carrier will typically be a liquid, such as sterile pyrogen-free water, sterile normal saline, hypertonic saline, pyrogen-free phosphate-buffered saline solution. For other methods of administration, the carrier can be either solid or liquid. For oral administration, the non-natural molecule can be administered in solid dosage forms, such as capsules, tablets, and powders, or in liquid dosage forms, such as elixirs, syrups, and suspensions. Non-natural molecules can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance. Formulations suitable for buccal (sub-lingual) administration include lozenges comprising the non-natural molecule in a flavored base, usually sucrose and acacia or tragacanth; and pastilles comprising the non-natural molecule in an inert base such as gelatin and glycerin or sucrose and acacia. Formulations of the present invention suitable for parenteral administration comprise sterile aqueous and non-aqueous injection solutions of the non-natural molecule, which preparations are preferably isotonic with the blood of the intended recipient. These preparations can contain antioxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a non-natural molecule of the invention, in a unit dosage form in a sealed container. The non-natural molecule or salt is provided in the form of a lyophilizate which is capable of being reconstituted with a suitable pharmaceutically acceptable carrier to form a liquid composition suitable for injection thereof into a subject. The unit dosage form typically comprises from about 1 mg to about 10 grams of the non-natural molecule or salt. When the non-natural molecule or salt is substantially water-insoluble, a sufficient amount of emulsifying agent which is pharmaceutically acceptable can be employed in sufficient quantity to emulsify the non-natural molecule or salt in an aqueous carrier. One such useful emulsifying agent is phosphatidyl choline. Formulations suitable for rectal administration are preferably presented as unit dose suppositories. These can be prepared by admixing the non-natural molecule with one or more conventional solid carriers, for example, cocoa butter, and then shaping the resulting mixture. Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof. Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986) and typically take the form of an optionally buffered aqueous solution of the peptides. Suitable formulations comprise citrate or bis/tris buffer (pH 6) or ethanol/water and contain from 0.1 to 0.2M of the compound. The non-natural molecule can alternatively be formulated for nasal administration or otherwise administered to the lungs of a subject by any suitable means, e.g., administered by an aerosol suspension of respirable particles comprising the non-natural molecule, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or another carrier gas, which can be delivered by insufflation from an inhaler device, for example. Aerosols of liquid particles comprising the non-natural molecule can be produced by any suitable means, such as with a pressure-driven aerosol nebulizer or an ultrasonic nebulizer, as is known to those of skill in the art. Aerosols of solid particles comprising the non-natural molecule can likewise be produced with any solid particulate medicament aerosol generator, by techniques known in the pharmaceutical art. Alternatively, one can administer the non-natural molecules in a local rather than systemic manner, for example, in a depot or sustained-release formulation.

Further, the present invention provides liposomal formulations of the non-natural molecules disclosed herein and salts thereof. The technology for forming liposomal suspensions is well known in the art. When the non-natural molecule or salt thereof is an aqueous-soluble salt, using conventional liposome technology, the same can be incorporated into lipid vesicles. In such an instance, due to the water solubility of the non-natural molecule or salt, the non-natural molecule or salt will be substantially entrained within the hydrophilic center or core of the liposomes. The lipid layer employed can be of any conventional composition and can either contain cholesterol or can be cholesterol-free. When the non-natural molecule or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques. The liposomal formulations containing the peptides disclosed herein or salts thereof, can be lyophilized to produce a lyophilizate which can be reconstituted with a pharmaceutically acceptable carrier, such as water, to regenerate a liposomal suspension. In the case of water-insoluble non-natural molecules, a pharmaceutical composition can be prepared containing the water-insoluble peptide, such as for example, in an aqueous base emulsion. In such an instance, the composition will contain a sufficient amount of pharmaceutically acceptable emulsifying agent to emulsify the desired amount of the non-natural molecule. Particularly useful emulsifying agents include phosphatidyl cholines and lecithin. In particular embodiments, the non-natural molecules is administered to the subject in a therapeutically effective amount, as that term is defined above. Dosages of pharmaceutically active non-natural molecules can be determined by methods known in the art, see, e.g., Remington's Pharmaceutical Sciences. The therapeutically effective dosage of any specific non-natural molecule will vary somewhat from non-natural molecules to non-natural molecule, and patient to patient, and will depend upon the condition of the patient and the route of delivery. As a general proposition, the total amount of the active ingredient to be administered will generally range from about 0.001 mg/kg to about 200 mg/kg body weight per day, and preferably from about 0.01 mg/kg to about 50 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight. Preferably, compositions for inhalation are presented for administration to the respiratory tract as a snuff or an aerosol or solution for a nebulizer, or as a microfine powder for insufflation, alone or in combination with an inert carrier such as lactose. In such a case the particles of active non-natural molecules suitably have diameters of less than 50 microns, preferably less than 10 microns, for example between 1 and 5 microns, such as between 2 and 5 microns. Alternatively, coated nanoparticles can be used, with a particle size between 30 and 500 nm. A favoured inhaled dose will be in the range of 0.05 to 2 mg, for example 0.05 to 0.5 mg, 0.1 to 1 mg or 0.5 to 2 mg.

It is evident for the skilled artisan that the specific initial and continuing dosage regimen for each patient will vary according to the nature and severity of the condition as determined by the attending diagnostician, the activity of the specific non-natural molecule employed, the age and general condition of the patient, time of administration, route of administration, rate of excretion of the drug, drug combinations, and the like. The desired mode of treatment and number of doses of a non-natural molecule of the present invention or a pharmaceutically acceptable salt or ester or composition thereof can be ascertained by those skilled in the art using conventional treatment tests.

As is common practice, the compositions will usually be accompanied by written or printed directions for use in the medical treatment concerned.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults.

Combination Therapies

In a particular embodiment the non-natural molecules of this invention can be administered as the sole pharmaceutical agent or in combination with one or more other pharmaceutical agents where the combination causes no unacceptable adverse effects. The present invention relates also to such combinations. For example, the non-natural molecules of this invention can be combined with known agents to reduce pathological aggregation as well as with admixtures and combinations thereof, or with known agents which induce the intracellular proteolytic system, in particular the autophagy system. Well-known examples of autophagy inducers are described in Russo M and Russo G L (2018), Biochem. Pharmacol. 153, 51-56, see Table 1; some examples are BH-3 mimetics ((−) gossypol, ABT-737, obatoclax mesylate and approved drugs such as metformin, rapamycin and rapalogs and natural compounds such as trehalos, resveratrol, curcumin and quercetin.

The term “treating” or “treatment” as stated throughout this document is used conventionally, e.g. the management or care of a subject for the purpose of combating, alleviating, reducing, relieving, improving the condition of, etc., of a disease or disorder, such as an amyloid disorder.

It is to be understood that although particular embodiments, specific configurations as well as materials and/or molecules, have been discussed herein for engineered peptides and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

1. Design of Capping Peptides

In the present invention we have developed a methodology for the structure-based design of capping peptide sequences, the latter can stop (or reduce) the aggregation of proteins which can form pathological aggregates in mammals. The method hinges on the availability of the 3D-structure of a pathological aggregate (a pathological aggregate is either the 3-D structure of the full-length protein capable of forming pathological aggregates or the 3-D structure of the amyloid core) of a protein which is capable of forming pathological aggregates. Although proteins forming pathological aggregates are categorized in literature (see Chiti F and Dobson C M (2017) Annu. Rev. Biochem. 86: 27-68, Table 1 and Tablet) as proteins capable of forming amyloid or non-amyloid fibrils, it should be said that also proteins which are listed as forming non-amyloid fibrils (such as for example FUS, p53 and TDP-43) have short aggregation protein regions which when isolated form amyloid fibrils and from these amyloid fibrils the amyloid core 3-D structure is available (further in short: the amyloid core structure). In our method starting from the 3-D structure of a pathological aggregate or the amyloid core structure of a protein capable of forming pathological aggregates, a forcefield algorithm is used to calculate the interaction energies between a list of candidate capping peptides (see further) and the amyloid core structure (or the 3-D structure of the pathological aggregate). In the present example we have used the FoldX force field to calculate the thermodynamic stability of the putative interactions. The template 3D-structures for a particular amyloid core structure can for example be retrieved from the on-line Protein Data Bank (PDB-database, (www.rcsb.org)).

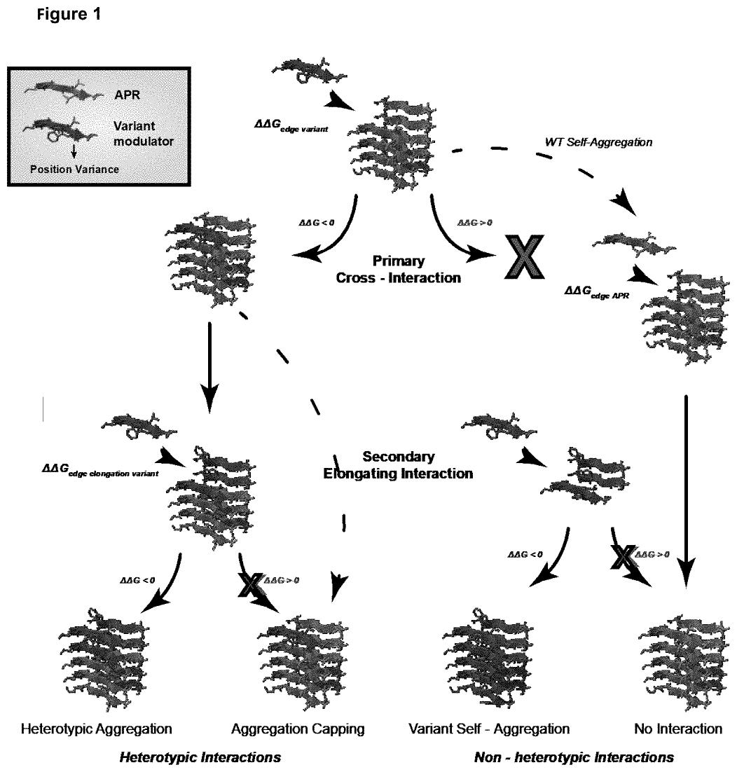

The first step in the methodology starts by generating an in silico list of variants of the amino acid sequence of the amyloid core. Amino acid sequences of the amyloid core are known as aggregation prone regions (APR regions) which are peptides of between 5 to 10 amino acids present in a protein capable of forming a pathological aggregate. APR sequences are the primary amino acid sequences of the amyloid core (for which a 3-D structure is available) or such APR sequences can be identified in a protein capable of forming pathological aggregates using a suitable APR-identification algorithm as described herein before (and a 3-D structure can be generated or can be predicted). Thus, starting from the APR sequence an in silico list of variants is created wherein one, two or three amino acids in this APR sequence are substituted into all possible 19 different amino acids (in FIG. 1 such variant is depicted as “edge variant”). In a subsequent step the candidate peptides (consisting of the in silico list of APR variants) are further used according to the methodology below.

In building our method we reasoned that for a candidate peptide to qualify as a capping peptide, it should strongly bind to the axial end of a growing amyloid core but at the same time the peptide should introduce sufficient structural disruption which prohibits further elongation along the fibril axis. The latter is in contrast to a wild type (or normal) elongating/nucleating sequence. The method below is illustrated with variants having one amino acid difference as compared to the sequence of the wild type APR region.

This requirement can be broken down to the combinatory outcome between two interaction energies (see FIG. 1):

ΔΔG_{cross-interaction}=ΔG_{edge variant}−ΔG_{edge APR} (1)

ΔΔG_{elongation}=ΔG_{edge elongation variant}−ΔG_{edge APR} (2)

where ΔG_{edge variant} is the interaction energy between a single variant chain docked against an APR amyloid core (FIG. 1), the ΔG_{edge elongation variant} is the free energy of interaction between a single variant chain docked against a variant-seeded axial end of the APR amyloid core (FIG. 1) and ΔG_{edge APR} corresponds to the interaction energy between a single APR chain against the same amyloid core (FIG. 1, APR self-aggregation pathway).

A favourable interaction is required from (1) in order for a sequence to be considered compatible to engage in cross-talk with an APR aggregation core. If the elongating energy of the same variant, given by the second function (2), corresponds to a simultaneous favourable interaction, then the variant can promote the heterotypic growth of the amyloid thus leading to a co-aggregating pathway (see FIG. 1, Heterotypic Aggregation). On the other hand, sequences which introduce steric disruptions at the edge of the fibril will result in blocking further growth on the tip and therefore act as aggregation inhibitors (see FIG. 1, Aggregation Capping).

The interaction potentials are computed in conjunction to the aggregating potential of additive APR events at the same end of the fibril. The reasoning behind this comparison is three-fold: (i) it provides direct comparison of the calculated energy potentials to thermodynamically stable interacting segments derived from experimentally determined crystal structures, (ii) it allows to compare the cross-talk potentials between alternate APR amyloid cores, as it is indifferent to the starting stability of the amyloid structure and (iii) the energies are calculated using the FoldX algorithm, which was initially built and excels in predicting the stabilising effects of mutations on protein or peptide stability.

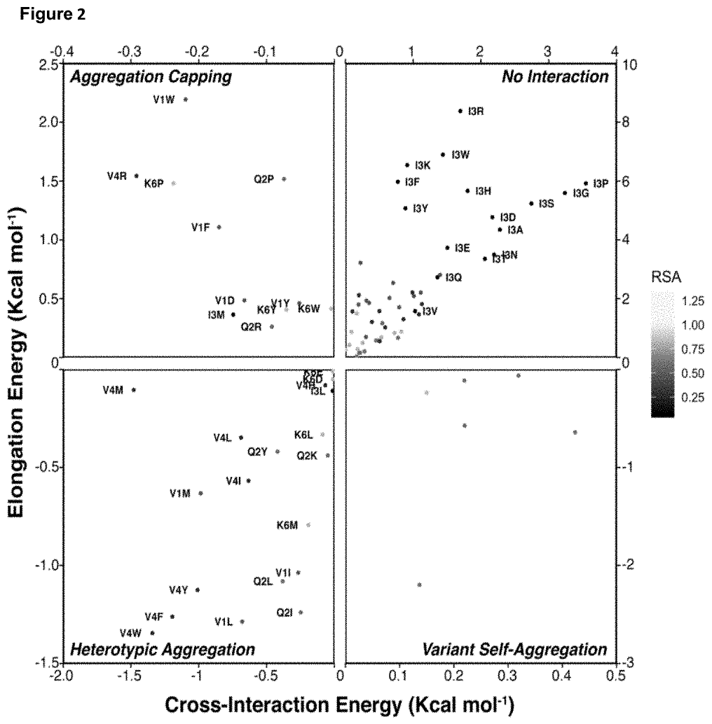

By plotting the interaction potential calculated through (1) on the x-axis and the potential from (2) on y-axis we end up with a quadratic profile of every of the variant sequences (see FIG. 2). FIG. 2 depicts amino acid sequence variants of SEQ ID NO: 8 which is an APR identified in the tau protein. The top left quadrant corresponds to sequence variants that are predicted to act as potential capping peptides against the identified APR template structure. A favorable variant sequence (in the top left quadrant) has a negative delta G free energy for cross interaction with the three-dimensional structure of the APR core and a positive delta G free energy for elongation with the three-dimensional structure of the APR core with a variant sequence bound to the axial end.

2.Design of Non-Natural Molecules which can Specifically Degrade Tau Amyloids

Table 2 depicts the elements to produce non-natural molecules to specifically degrade tau amyloids. The first column depicts the ID name of the capping peptide molecules. The second column (titled capping peptides) depicts single capping peptides and tandem capping peptides. In tandem capping peptides two identical or two different capping peptides can be used. Capping peptides are designed to stop tau aggregate formation. All capping peptides shown in Table 2 are based on two APRs identified in the tau protein: ₆₂₃VQIVYK₆₂₈ (SEQ ID NO: 7) and ₅₉₂VQIINK₅₉₇ (SEQ ID NO: 8). Since the capping peptides (depicted in column 2) do not degrade pathological tau amyloids, the capping peptides are fused to a moiety targeting the intracellular proteolytic system. Fusion to a VHL-tag (column 3, ALAOYIP peptide sequence (SEQ ID NO: 1), “O” is the amino acid hydroxyproline) or fusion to a Javelin-tag (column 3, NLLRLTGW peptide sequence (SEQ ID NO: 6)) or fusion to a small molecule (pomalidomide) which targets Cereblon (CRBN). The combination between the capping peptides depicted in Table 2 (11 different peptide sequences in column 1) and the respective 3 different targeting moieties (moieties depicted in respective columns 3, 4 and 5) leads to a combination of 33 different non-natural molecules. In the tandem capping peptides (between the same or different capping peptides) and between the (tandem) capping peptides and the VHL- or Javelin-tag a flexible GS-linker is inserted.

TABLE 2

| | | | | Small molecule |
|---|---|---|---|---|
| | | peptide | peptide | moiety |
| | | sequence | sequence | targeting |
| | | of the | of the | Cereblon |
| ID | Capping peptides | VHL-moiety | Javelin-moiety | (CRBN) |
| (column 1) | (column 2) | (column 3) | (column 4) | (column 5) |
| CAP1 | WQIVYK | -GSGSALAQYIP | -GSGSNLLRLTGW | -pomalidomide |
| | (SEQ ID NO: 9) | (SEQ ID NO: 10) | (SEQ ID NO: 11) | |
| CAP1_T | WQIVYKGSWQIVYK | -GSGSALAQYIP | -GSGSNLLRLTGW | -pomalidomide |
| | (SEQ ID NO: 12) | (SEQ ID NO: 10) | (SEQ ID NO: 11) | |
| CAP1_TR | RWQIVYKEGSRWQIVYKR | -GSGSALAQYIP | -GSGSNLLRLTGW | -pomalidomide |
| | (SEQ ID NO: 13) | (SEQ ID NO: 10) | (SEQ ID NO: 11) | |
| CAP1_TD | DWQIVYKDGSDWQIVYKD | -GSGSALAQYIP | -GSGSNLLRLTGW | -pomalidomide |
| | (SEQ ID NO: 14) | (SEQ ID NO: 10) | (SEQ ID NO: 11) | |
| CAP2 | VQIVYP | -GSGSALAQYIP | -GSGSNLLRLTGW | -pomalidomide |
| | (SEO ID NO: 15) | (SEQ ID NO: 10) | (SEQ ID NO: 11) | |
| CAP2_T | VQIVYPGSVQIVYP | -GSGSALAQYIP | -GSGSNLLRLTGW | -pomalidomide |
| | (SEO ID NO: 16) | (SEQ ID NO: 10) | (SEQ ID NO: 11) | |
| CAP2_TR | RVQIVYPRGSRVQIVYPS | -GSGSALAQYIP | -GSGSNLLRLTGW | -pomalidomide |
| | (SEQ ID NO: 17) | (SEQ ID NO: 10) | (SEQ ID NO: 11) | |
| CAP2_TD | QVQIVYPDGSDVQIVYPD | -GSGSALAQYIP | -GSGSNLLRLTGW | -pomalidomide |
| | (SEQ ID NO: 18) | (SEQ ID NO: 10) | (SEQ ID NO: 11) | |
| HET | WQIVYKGSVWMINK | -GSGSALAQYIP | -GSGSNLLRLTGW | -pomalidomide |
| | (SEQ ID NO: 19) | (SEQ ID NO: 10) | (SEQ ID NO: 11) | |
| HET_R | WQIVYKRGSRVWMINKR | -GSGSALAQYIP | -GSGSNLLRLTGW | -pomalidomide |
| | (SEQ ID NO: 20) | (SEQ ID NO: 10) | (SEQ ID NO: 11) | |
| HET_D | QWQIVYKDGSDVWMINKD | -GSGSALAQYIP | -GSGSNLLRLTGW | -pomalidomide |
| | (SEQ ID NO: 21) | (SEQ ID NO: 10) | (SEQ ID NO: 11) | |

schematic overview of the different capping peptides (capping peptide sequences are underlined in column 2) and combination with 3 different moieties (columns 3, 4 and 5) which leads to 33 different non-natural molecules of the invention which are used in the experiments.
Residues R and D are gatekeeper amino acids, residue 0 is the artificial amino acid hydroxyproline.
GS and GSGS are linker sequences.

3.Cellular Screening Assay of the Non-Natural Molecules

The 33 different non-natural molecules (with a final concentration of 10 μM) were mixed with preformed (sonicated) tau aggregates (with a final concentration of 4 μM). The preformed tau aggregates are labeled with a far-red atto-633 dye to monitor the efficiency of cellular uptake and degradation. After an incubation of 6 hours the resulting non-natural molecule—tau aggregate mixture was transfected in tau biosensor cells (described in Holmes et al (2014) Proc Natl Acad Sci USA. 111(41): E4376-E4385). The tau biosensor cell line is a monoclonal FRET biosensor HEK293T cell line stably expressing both the tau repeat domain (RD) fused to the fluorescent protein CFP and the tau repeat protein fused to the fluorescent protein YFP. Without the addition of preformed tau seeds, the biosensor cells stably express diffuse tau (soluble). However, exposure to exogenous preformed tau aggregates induces tau aggregation, generating a FRET signal with the CFP-YFP pair, intensifying the fluorescent signal in the tau inclusions. The FRET signal allows to distinguish the tau aggregate formation from the monomeric background. The use of phospholipids (e.g. lipofectamine—Source: Thermofisher) for the transfection of exogenous tau aggregates maximizes the aggregate inducing capacity.

The final peptide concentration of the non-natural molecules applied on the cells is 500 nM and the final concentration of preformed tau aggregates is 200 nM. 20 hours after transfection cells were fixed and two readouts were used to quantify the activities of the non-natural molecules:
determination of the fraction of cells with green spots: these are the cells that have induced tau aggregation.
determination of the fraction of cells with red spots: these are the cells that are positive for preformed tau aggregates.

It's important to note that this screen is performed at only one non-natural molecule concentration (500 nM) which is not ideal for comparing the activity of the non-natural molecules. Indeed, a too high concentration of a degradation inducing molecule can have a negative effect on its activity (biphasic behavior). This assay therefore serves as a first filter to identify active (or inactive) non-natural molecules as defined in the instant invention.

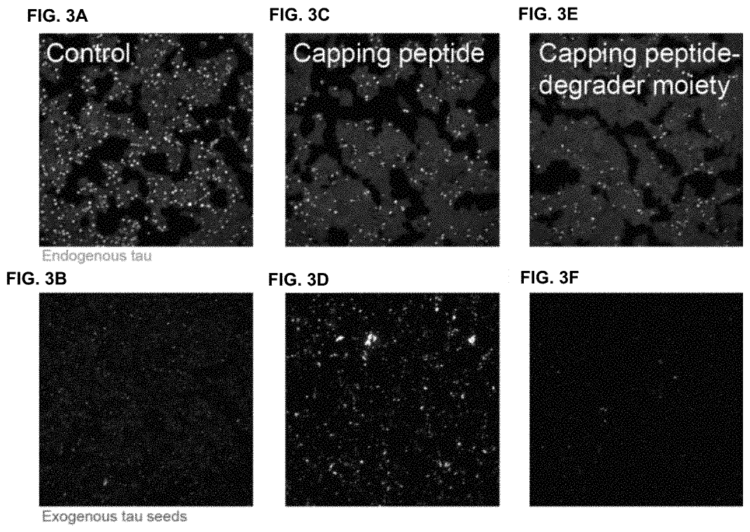

FIG. 3 depicts the general principle of the tau biosensor cells treated with a vehicle control, a capping peptide (see column 2) and a non-natural molecule (capping peptide fused to a degradation moiety).

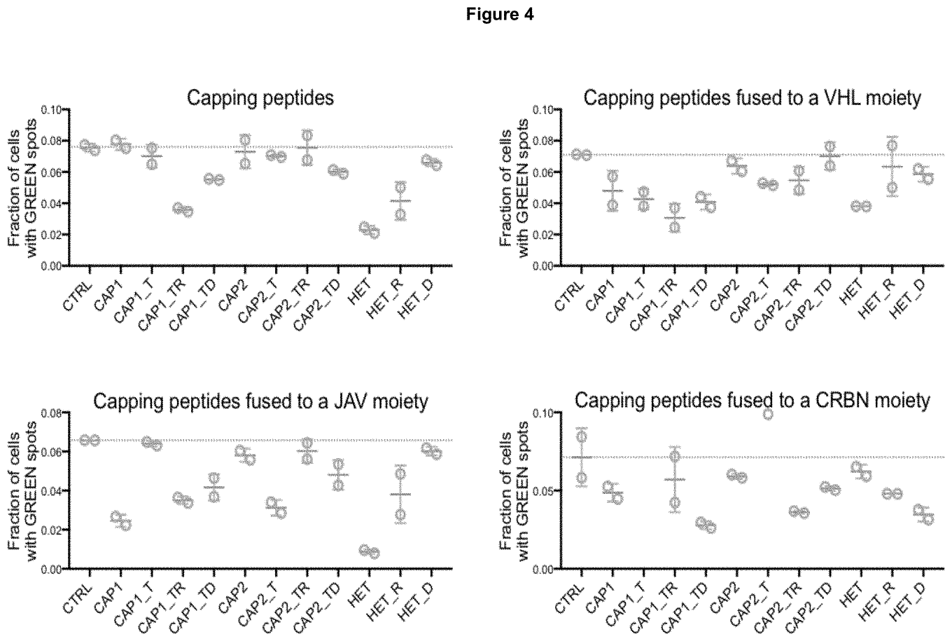

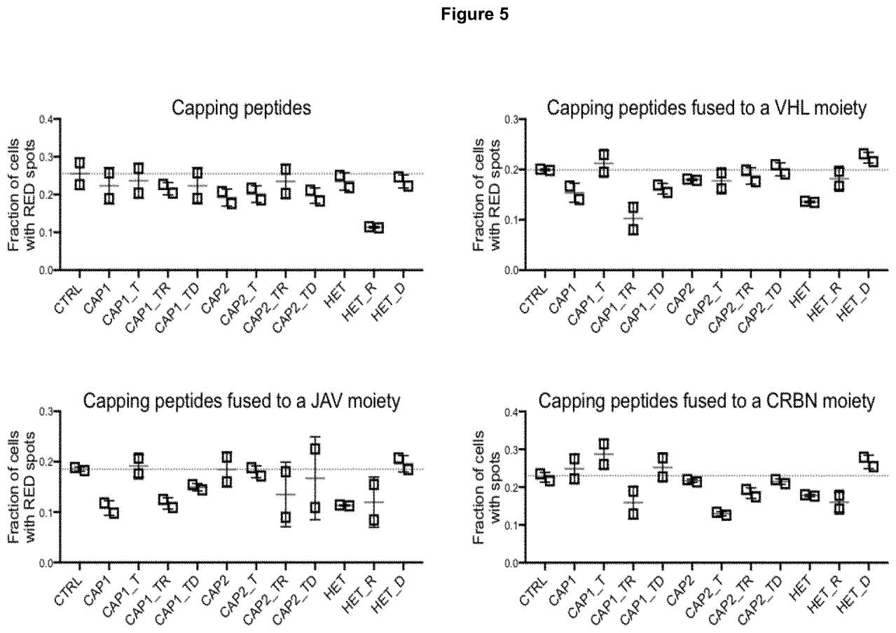

In a next step all the non-natural molecules depicted in Table 2 (combinations of the peptides of column 2 with the respective moieties depicted in columns 3, 4 and 5) were screened with the tau biosensor seeding assay (see FIGS. 4 and 5). Data analysis is based on the quantification of the fraction of cells that contain green (endogenous tau aggregation) — see FIG. 4 or red (exogenous tau aggregates) spots—see FIG. 5.

Two capping peptides were selected for further producing non-natural molecules in follow up experiments: CAP1_TR and HET (see ID numbers in column 1 of Table 2). Although the latter capping peptides are tandem peptides, non-natural molecules based on the sequence of the ‘single’ peptide CAP1 also show capping activity (capping peptides fused to a JAV moiety, see FIG. 2) and degrading activity (capping peptides fused to a JAV moiety, see FIG. 5).

4. Cellular Assay—Optimization of Concentration Ranges

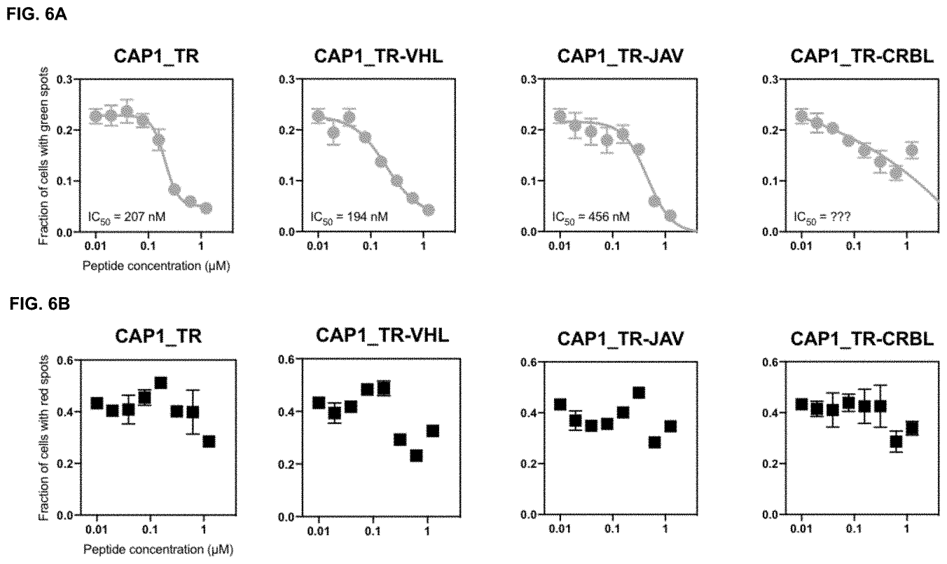

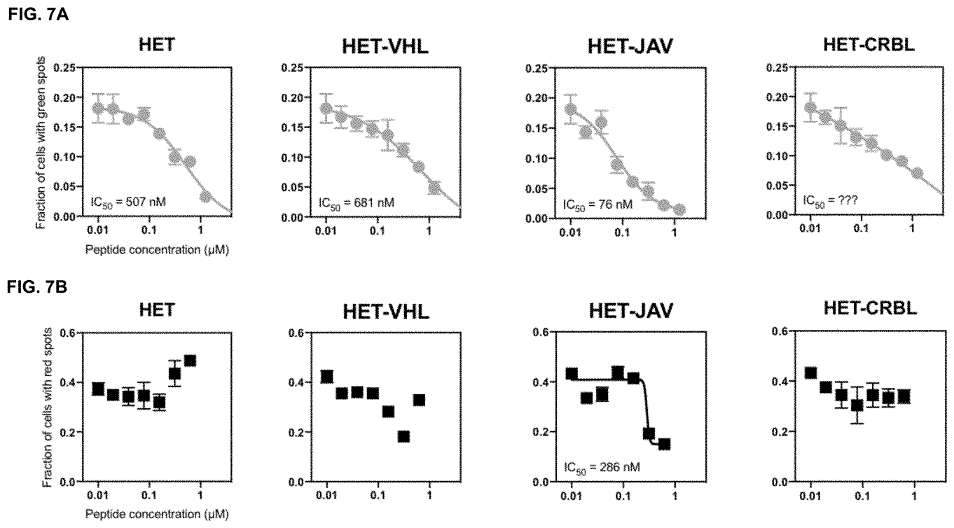

In this example non-natural molecules of the invention were generated based on the sequences of capping peptides CAP1_TR and HET (sequences depicted in column 2 in Table 2). A tau biosensor seeding assay was performed with a concentration range of peptides. Thus, the assay is the same as described in example 2 but now a range of concentrations of capping peptides and non-natural molecules comprising the capping peptides were used to incubate with preformed tau aggregates. FIGS. 6 and 7 depict the final concentration of the peptides and derived non-natural molecules applied on the cells (see FIGS. 6 and 7).

FIG. 6 shows that each of the non-natural molecules comprising the CAP1_TR peptide reduce induced tau aggregation in a concentration dependent manner (see curves in FIG. 4A). Moreover, the non-natural molecule comprising the VHL-moiety induces tau aggregate degradation at the highest concentrations (see curves in FIG. 4B). It is observed that the naked capping peptide CAP1_TR (id est not fused to a degradation moiety) also shows a slight reduction in cells positive for red spots in the highest concentration. This is not due to degradation, but due to reduced uptake (the red tau aggregates are stuck on the outside of the cell). This is a technical artifact but even with this artifact it is clear that the non-natural molecule comprising the VHL-moiety reduces the fraction of cells positive for tau aggregates even more.

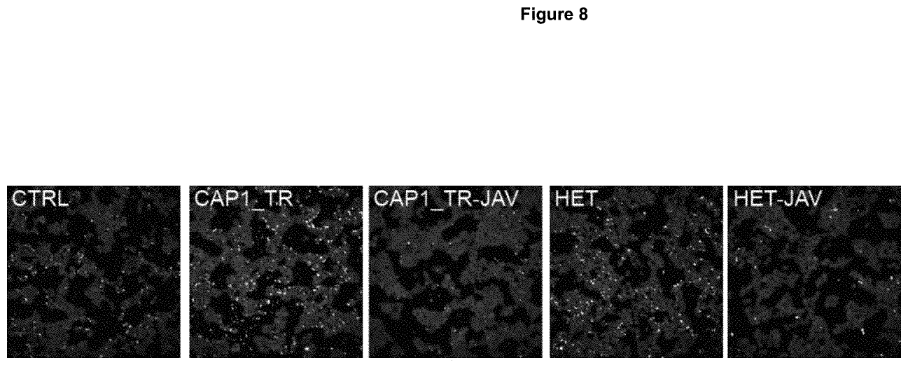

FIG. 7 shows that each of the non-natural molecules comprising the HET peptide and a degradation moiety reduce induced tau aggregation in a concentration dependent manner (green curves in FIG. 7A). Moreover, the non-natural molecules comprising the VHL- and JAV-moieties induce tau aggregate degradation at the highest concentrations (red curves in FIG. 7B). The non-natural molecule comprising the VHL-moiety shows a biphasic curve, indicating that at a higher concentration of this non-natural molecule, the cellular degradation system gets overloaded. The non-natural molecule comprising a JAV-moiety shows a clear concentration dependent degradation of tau aggregates. As a further example, FIG. 8 represents the effect of peptides and non-natural molecules derived thereof CAP1_TR, CAP1_TR-JAV, HET and HET-JAV (at 312 nM) on tau biosensor cells seeded with preformed tau aggregates.

We would like to note that at first sight the non-natural molecule CAP1_TR-JAV does not show a clear effect on tau aggregate degradation (at the applied concentration) according to the quantifications shown above (see FIG. 6B, third panel) because we observe that compared to the control, the same number of cells still contain atto-633 tau aggregates. Nevertheless, FIG. 8 (third panel) clearly shows an effect on atto-633 tau aggregate size inside the cells (cells only contain very small red inclusions). One possible explanation is that tau degradation is indeed ongoing but did not reach its the final stage yet.

5. In Vitro Tau Aggregation Assay

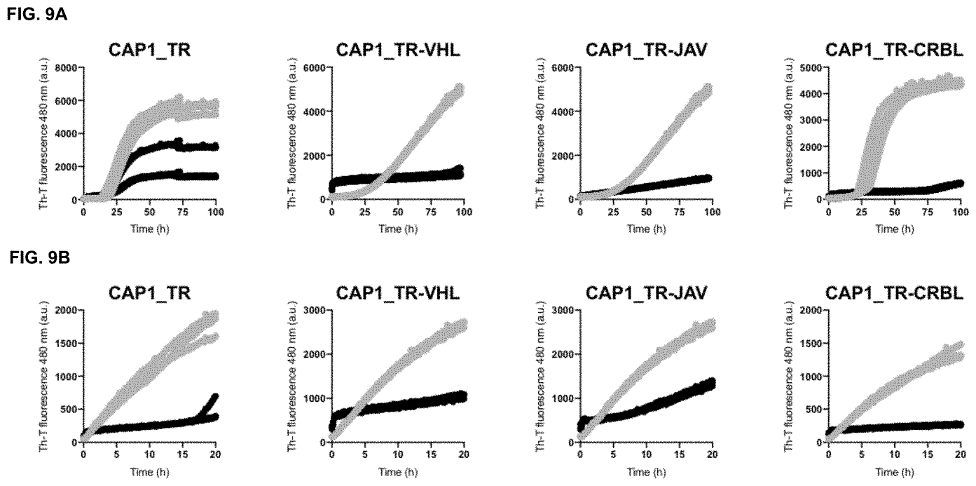

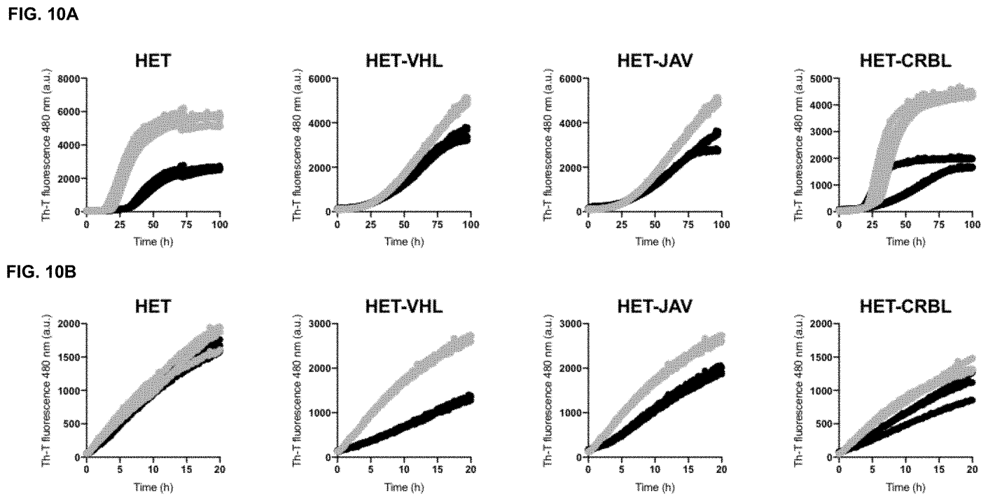

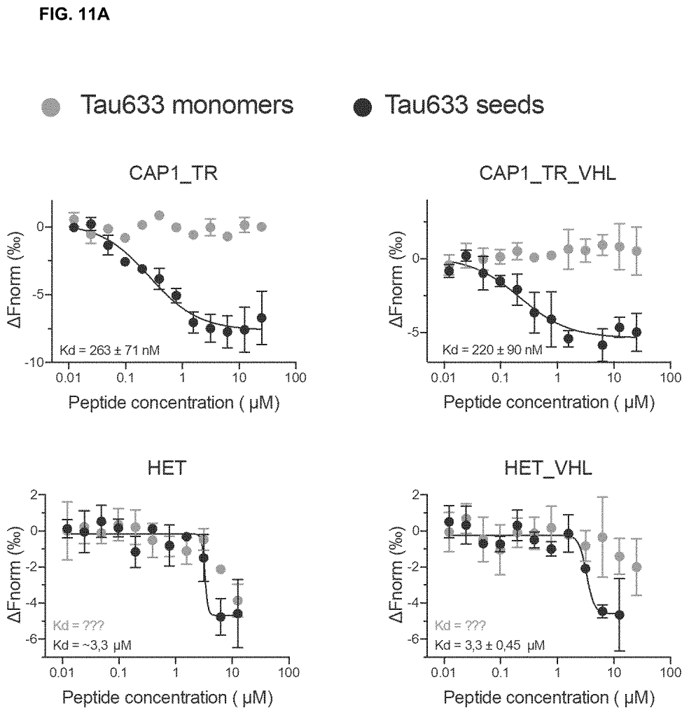

To validate whether the capping peptides and non-natural molecules derived thereof also reduce tau aggregation in vitro, we performed a Th-T aggregation assay (Xue et al (2017) R Soc Open Sci. 4(1):160696). Capping peptides (with a final concentration of 6 μM) were mixed with full-length monomeric tau protein (at a final concentration of 9 μM) and Th-T fluorescence was monitored over time. The was performed without the addition of preformed tau aggregates (‘non-seeded’) and with the addition of preformed tau aggregation (‘seeded’). In the latter case, preformed tau-aggregates were pre-incubated with the capping peptides and non-natural molecules derived thereof for 2 hours prior to assay initiation. The data are shown in FIGS. 9 and 10.

6. Affinity Calculations of the Non-Natural Molecules of the Invention

To measure the affinity of the capping peptides, and non-natural molecules derived thereof, with full length tau, we performed Microscale Thermophoresis (MST, Monolith NT.Automated) experiments. Briefly, atto-633-labeled tau monomers or atto-633-labeled preformed tau aggregates were mixed with a concentration range of capping peptides, and non-natural molecules derived thereof, and MST measurement were performed.

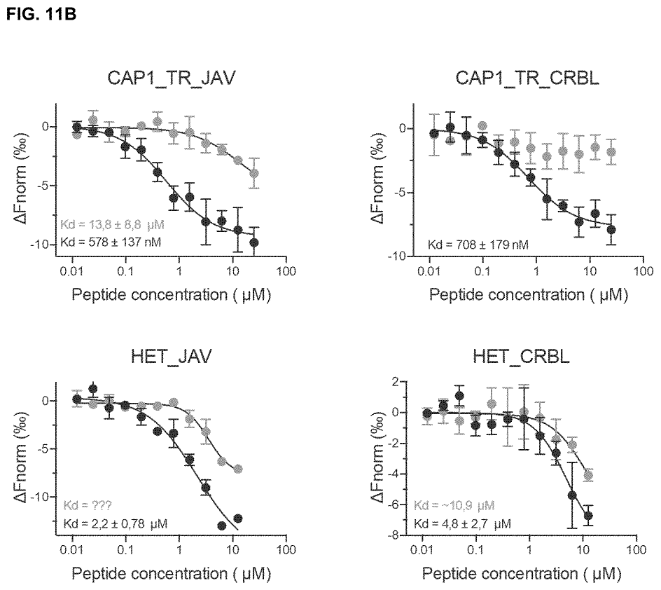

The data for all non-natural molecules comprising the CAP1_TR capping peptide is very clear (see FIG. 11A). Indeed, nanomolar affinities are obtained for tau aggregates while no binding is observed to tau monomers. The HET capping peptide and non-natural molecules derived thereof also show a higher affinity for tau aggregates compared to tau-monomers (see FIG. 11B), however the measured affinities are significantly higher compared to CAP1_TR variants. This could be due to the nature of MST measurements, which depends on the actual effect of peptide binding on the movement of tau aggregates. Affinity data can also be measured by using Biolayer Interferometry (BLI). The latter technology allows to calculate accurate Kₒₙ and K_{off} rates.

7. Efficiency of Uptake of the Non-Natural Molecules of the Invention

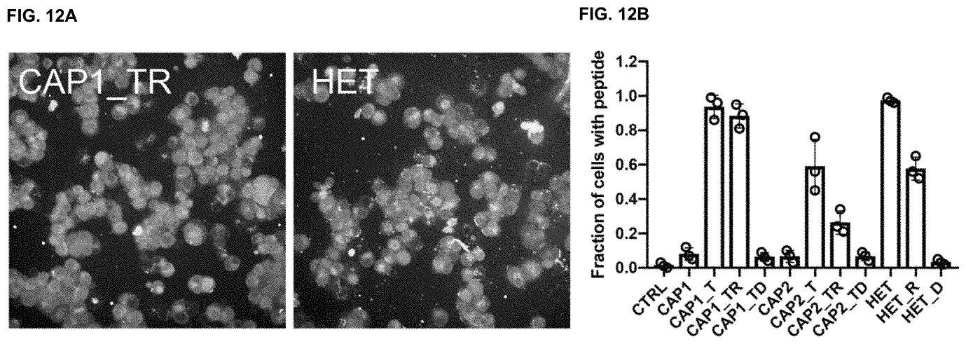

To monitor cellular uptake of the non-natural molecules of the invention (without using lipofectamine) fluorescently labeled variants were used. As pomalidomide shows intrinsic green fluorescence (excitation=440 nm; emission=510 nm), non-natural molecules comprising pomalidomide were used. Regular HEK293T cells were treated with a final concentration of 5 μM of each non-natural molecule and after 20 hours of incubation the non-natural molecule uptake was monitored by tracking pomalidomide fluorescence inside the cells (see FIG. 12).

We conclude that most of the non-natural molecules are efficiently taken up by cells albeit large differences are observed. We observed that non-natural molecules comprising tandem capping peptides have an improved cellular uptake, non-natural molecules without gatekeepers have a similar uptake efficiency as non-natural molecules comprising gatekeepers. Remarkably non-natural molecules comprising gatekeepers consisting of the amino acid D are less efficiently taken up by cells.

8. Specificity of the Non-Natural Molecules for Amyloid Forms

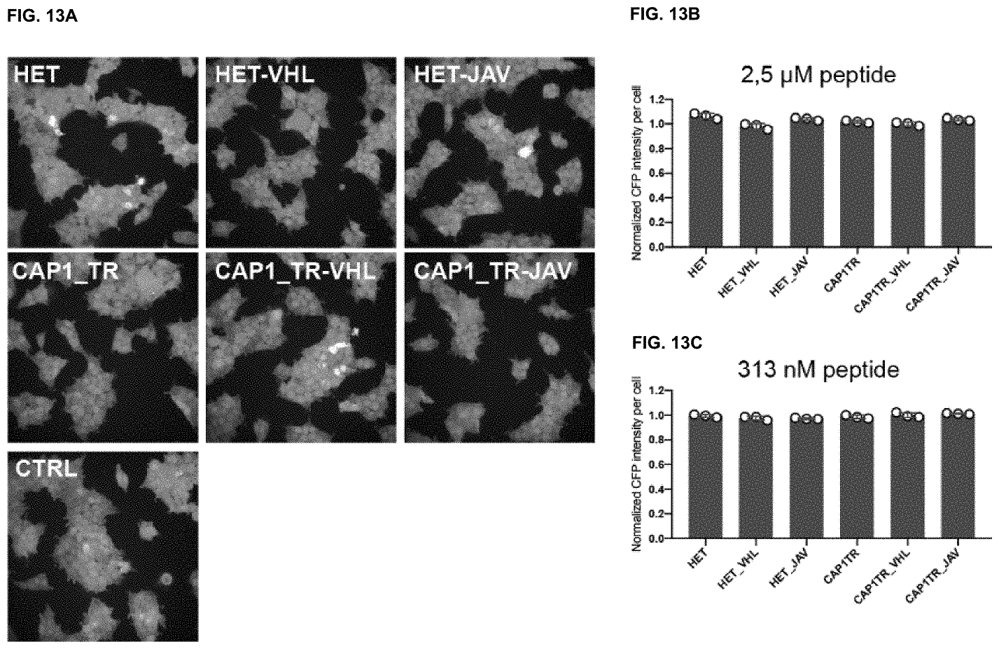

In this experiment we prove that the non-natural molecules of the invention do not degrade monomeric forms of tau. Thereto the biosensor cell line as outlined in example 2 is used. Capping peptides and non-natural molecules comprising capping peptides and degrader-moieties are transfected into the tau biosensor cell line without the addition of preformed tau seeds. After 20h of incubation, cells are fixed and checked for intrinsic tau-CFP intensity levels. FIG. 13 shows that none of the capping (degrader) peptides shows a change in fluorescence upon peptide treatment because monomeric tau is not degraded.

9. Non-Natural Mmolecules Comprising CMA-Inducing Moieties

In a next step we tested different non-natural molecules based on the capping peptide depicted in SEQ ID NO: 19 (see Table 2, HET-peptide). One non-natural molecule (HET_JAV, SEQ ID NO: 22) was based on fusion with the javelin moiety, a second and a third non-natural molecule was based on respective moieties CMA1 (SEQ ID NO: 23) and CMA2 (SEQ ID NO: 24).

| | |
|---|---|
| | (SEQ ID NO: 19) |
| HET | WQIVYKGSVWMINK |
| | (SEQ ID NO: 22) |
| HET_JAV | WQIVYKGSVWMINKGSGSNLLRLTGW |
| | (SEQ ID NO: 23) |
| HET_CMA1 | WQIVYKGSVWMINKGSGSKFERQKILDQRFFE |
| | (SEQ ID NO: 24) |
| HET_CMA2 | WQIVYKGSVWMINKGSGSVKKDQGSKFERQ |

Cellular screening of these non-natural molecules was conducted in accordance with example 3. FIG. 14 depicts the fraction of cells with green spots (FIG. 14A) and the fraction of cells with red spots (FIG. 14B).

10. Design of Tau Capping Peptides having 2 Amino Acid Variations in the Tau-APR Sequence

In the present example we designed capping peptides with a specificity for pathological Tau aggregates wherein the capping peptides have two amino acid mutations as compared to the selected APR sequences. Thereto lists of all possible 2 amino acid substitutions in SEQ ID NO: 7 and SEQ ID NO: 8 were generated. The selection of capping peptides from this double mutant lists was based on the method as described in example 1. The sequences of the obtained capping peptides for this approach are depicted in Table 3.

TABLE 3

| Target APR in Tau | Obtained capping peptide |
|---|---|
| VQIINK (SEQ ID NO: 8) | EQIINE (SEQ ID NO: 25) |
| VQIINK (SEQ ID NO: 8) | VQWIIK (SEQ ID NO: 26) |
| VQIINK (SEQ ID NO: 8) | EDIINK (SEQ ID NO: 27) |
| VQIINK (SEQ ID NO: 8) | VDIIDK (SEQ ID NO: 28) |
| VQIINK (SEQ ID NO: 8) | EDIINK (SEQ ID NO: 29) |
| VQIVYK (SEQ ID NO: 7) | DQIFYK (SEQ ID NO: 30) |
| VQIVYK (SEQ ID NO: 7) | DQIMYK (SEQ ID NO: 31) |
| VQIVYK (SEO ID NO: 7) | DQIWYK (SEQ ID NO: 32) |
| VQIVYK (SEQ ID NO: 7) | YQIYYK (SEQ ID NO: 33) |
| VQIVYK (SEQ ID NO: 7) | WQIWYK (SEQ ID NO: 34) |
| VQIVYK (SEQ ID NO: 7) | WQIYYK (SEQ ID NO: 35) |

capping peptide sequences with a specificity for tau pathological aggregates having 2 amino acid mutations as compared to respectively SEQ ID NO: 7 and SEQ ID NO: 8.

11. Tandem Capping Peptides Directed to Tau Pathological Aggregates

In the present example a screening was conducted with an aim to identify the most optimal hetero-dimeric tandem capping peptides for tau specificity. Hetero-dimeric tandem capping peptides consist of two single capping peptides, each targeting a different Tau APR sequence.

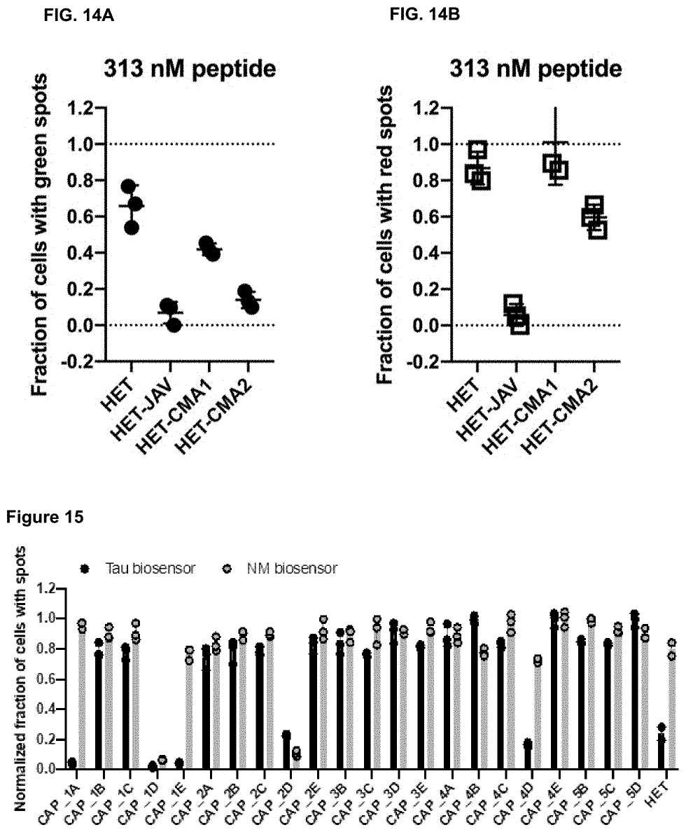

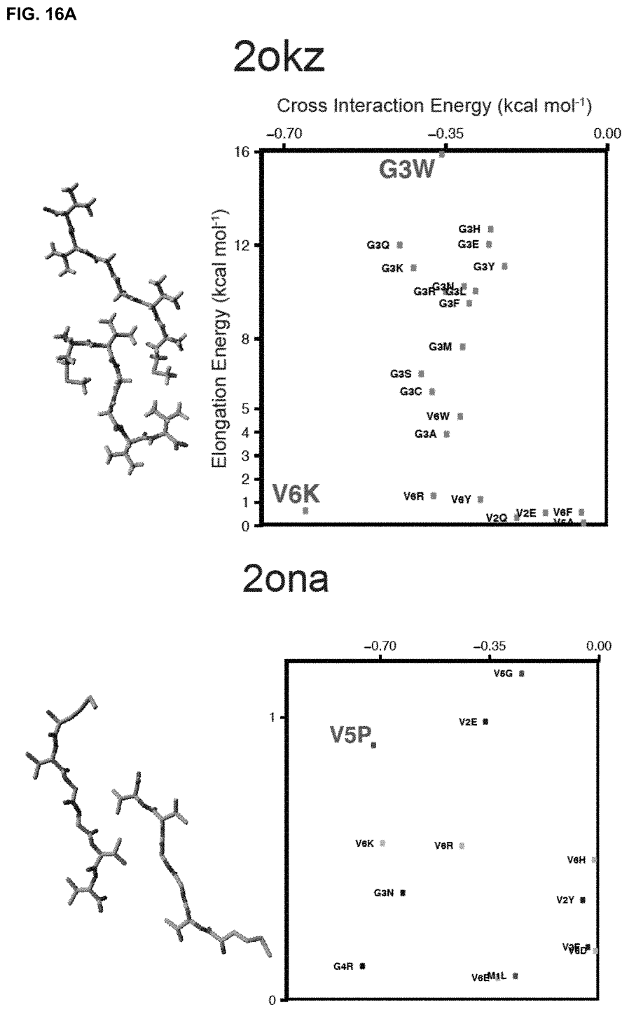

23 different hetero-dimeric tandem capping peptides targeting Tau were synthesized and the sequences are depicted in Table 4. These capping peptides were dissolved in DMSO, mixed with sonicated preformed Tau aggregates or Sup35-NM aggregates and transfected into a Tau biosensor cell line, expressing CFP- and YFP- labeled Tau repeat domain and a NM biosensor cell line, expressing GFP-labeled Sup35-NM, respectively. The Sup35-NM was biosensor line was used as cell line to detect a-specific effects of the hetero-tandem capping peptides. Indeed, the prion-determining region (NM) of the Saccharomyces cerevisiae translation termination factor Sup35 assembles into amyloid-like fibrils (see Hess S. et al (2007) EMBO reports 8, 12, 1196). The final concentration of capping peptide on cells was 625 nM and the final concentration of seed aggregates (Tau- or NM-seeds) was 50 nM. After 24 hours of incubation, the fraction of cells with induced aggregates was determined and normalized. The condition is which cells were treated with preformed aggregates without capping peptide was set at 1, while the condition in which cells were not treated with preformed aggregates was set at 0. The results in FIG. 15 show the average of 3 independent repeats (each consisting of 3 technical replicates). The data show that CAP_1A, CAP_1E and CAP_4D are the most performant peptides and are also specific for inhibiting tau aggregation.

TABLE 4

| ID of hetero- | Amino acid sequence | |
|---|---|---|
| dimeric Tau | of hetero-dimeric | Sequence |
| capping peptide | capping peptide | identifier |
| CAP_1A | VQWINKGSWQIVYK | SEQ ID NO: 36 |
| CAP_1B | VQWINKGSVOIVYP | SEQ ID NO: 37 |
| CAP_1C | VQWINKGSVPIVYK | SEQ ID NO: 38 |
| CAP_1D | VQWINKGSFQIVYK | SEQ ID NO: 39 |
| CAP_1E | VQWINKGSVQMVYK | SEQ ID NO: 40 |
| CAP_2A | VDIINKGSWQIVYK | SEQ ID NO: 41 |
| CAP_2B | VDIINKGSVQIVYP | SEQ ID NO: 42 |
| CAP_2C | VDIINKGSVPIVYK | SEQ ID NO: 43 |
| CAP_2D | VDIINKGSFQIVYK | SEQ ID NO: 44 |
| CAP_2E | VDIINKGSVQMVYK | SEQ ID NO: 45 |
| CAP_3B | VOIIDKGSVQIVYP | SEQ ID NO: 46 |
| CAP_3C | VQIIDKGSVPIVYK | SEQ ID NO: 47 |
| CAP_3D | VOIIDKGSFQIVYK | SEQ ID NO: 48 |
| CAP_3E | VOIIDKGSVQMVYK | SEQ ID NO: 49 |
| CAP_4A | EQIINKGSWQIVYK | SEQ ID NO: 50 |
| CAP_4B | EQIINKGSVQIVYP | SEQ ID NO: 51 |
| CAP_4C | EQIINKGSVPIVYK | SEQ ID NO: 52 |
| CAP_4D | EQIINKGSFQIVYK | SEQ ID NO: 53 |
| CAP_4E | EQIINKGSVQMVYK | SEQ ID NO: 54 |
| CAP_5B | VQIINGGSVQIVYP | SEQ ID NO: 55 |
| CAP_5C | VQIINGGSVPIVYK | SEQ ID NO: 56 |
| CAP_5D | VQIINGGSFQIVYK | SEQ ID NO: 57 |
| HET | WQIVYKGSVWMINK | SEQ ID NO: 19 |

sequences of the hetero-dimeric tandem Tau capping peptides

12. Design of Capping Peptides Directed to Amyloid-Beta

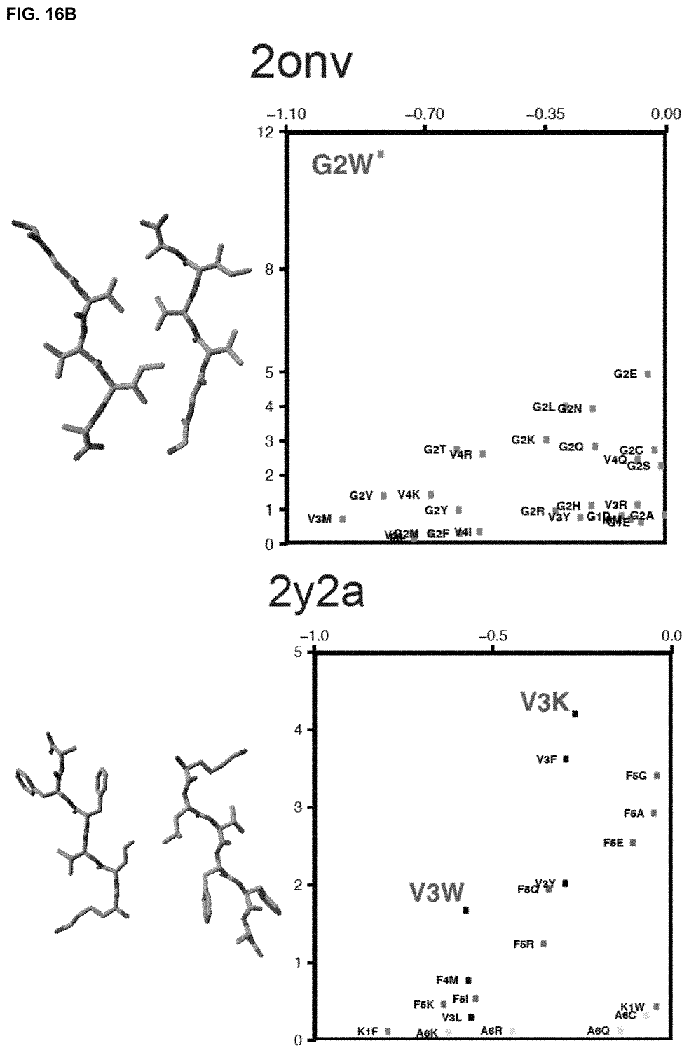

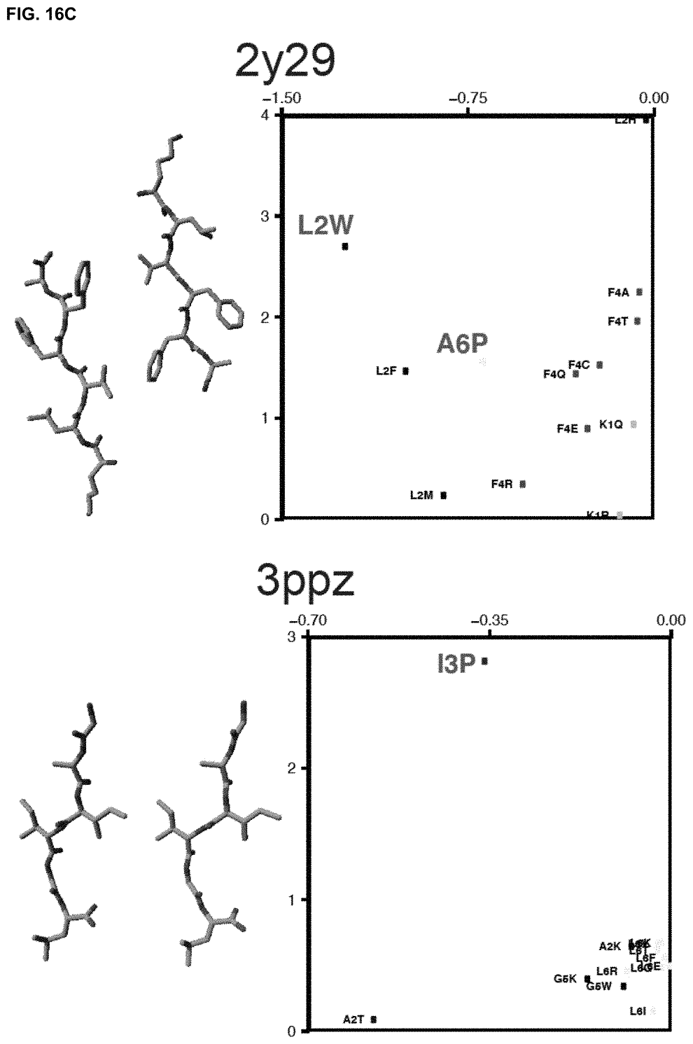

Capping peptides were designed based on the different amyloid structures available for all Aβ APR sequences (see FIG. 16). The single mutants with the most unfavorable elongation energy and the most favorable cross interaction energy were selected for capping peptide design and these are highlighted in bold and larger font size in FIG. 16.

Based on the in silico predicted capping peptide sequences in FIG. 16, we designed hetero-tandem capping peptides. Every hetero-tandem consist of two single capping peptide sequences, each targeting a different Aβ APR. The resulting sequences of these hetero-tandem capping peptides are depicted in Table 5.

TABLE 5

| ID | SEQUENCE |
|---|---|
| AbCap_1 | MVWGVVGSGWVVIA (SEQ ID NO: 63) |
| AbCap_2 | MVWGVVGSKLKFFA (SEQ ID NO: 64) |
| AbCap_3 | MVWGVVGSKLWFFA (SEQ ID NO: 65) |
| AbCap_4 | MVWGVVGSKWVFFA (SEQ ID NO: 66) |
| AbCap_5 | MVWGVVGSKWVFFP (SEQ ID NO: 67) |
| AbCap_6 | MVWGVVGSGAPIGL (SEQ ID NO: 68) |
| AbCap_7 | MVGGVKGSGWVVIA (SEQ ID NO: 69) |
| AbCap_8 | MVGGVKGSKLKFFA (SEQ ID NO: 70) |
| AbCap_9 | MVGGVKGSKLWFFA (SEQ ID NO: 71) |
| AbCap_10 | MVGGVKGSKWVFFA (SEQ ID NO: 72) |
| AbCap_ll | MVGGVKGSKWVFFP (SEQ ID NO: 73) |
| AbCap_12 | MVGGVKGSGAPIGL (SEQ ID NO: 74) |
| AbCap_13 | MVGGPVGSGWVVIA (SEQ ID NO: 75) |
| AbCap_14 | MVGGPVGSKLKFFA (SEQ ID NO: 76) |
| AbCap_15 | MVGGPVGSKLWFFA (SEQ ID NO: 77) |
| AbCap_16 | MVGGPVGSKWVFFA (SEQ ID NO: 78) |
| AbCap_17 | MVGGPVGSKWVFFP (SEQ ID NO: 79) |
| AbCap_18 | MVGGPVGSGAPIGL (SEQ ID NO: 80) |
| AbCap_19 | GWVVIAGSKLKFFA (SEQ ID NO: 81) |
| AbCap_20 | GWVVIAGSKLWFFA (SEQ ID NO: 82) |
| AbCap_21 | GWVVIAGSKWVFFA (SEQ ID NO: 83) |
| AbCap_22 | GWVVIAGSKWVFFP (SEQ ID NO: 84) |
| AbCap_23 | GWVVIAGSGAPIGL (SEQ ID NO: 85) |
| AbCap_24 | KLKFFAGSGAPIGL (SEQ ID NO: 86) |
| AbCap_25 | KLWFFAGSGAPIGL (SEQ ID NO: 87) |
| AbCap_26 | KWVFFAGSGAPIGL (SEQ ID NO: 88) |
| AbCap_27 | KWVFFPGSGAPIGL (SEQ ID NO: 89) |

sequences of AP hetero-tandem capping peptides used in the present invention

12.1 Aβ Biosensor Screening Assay

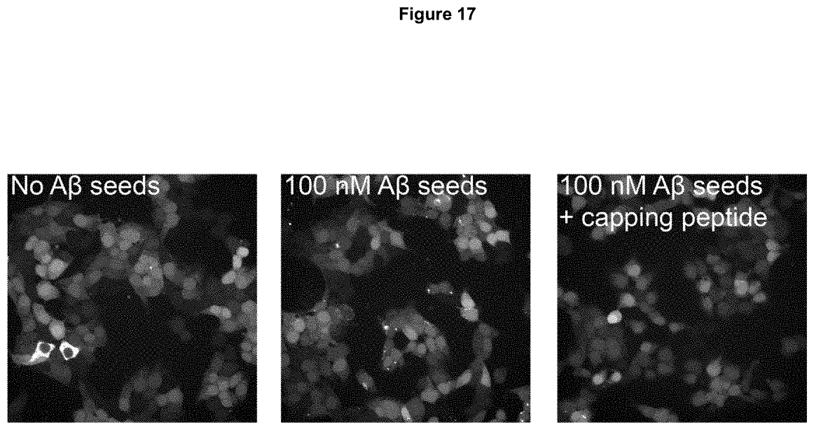

Hetero-tandem capping peptides depicted in Table 5 were dissolved in DMSO, filtered and mixed with preformed, sonicated Aβ aggregates (2 μM). This mixture was incubated for approx. 4 hours and transfected into Aβ biosensor cells. Aβ biosensor cells stably express soluble Aβ-mCherry and Aβ aggregation can be induced by transfecting preformed Aβ aggregates in these cells (FIG. 17). The final concentration of Aβ aggregates on the cells is 100 nM and a concentration range of capping peptides was used.

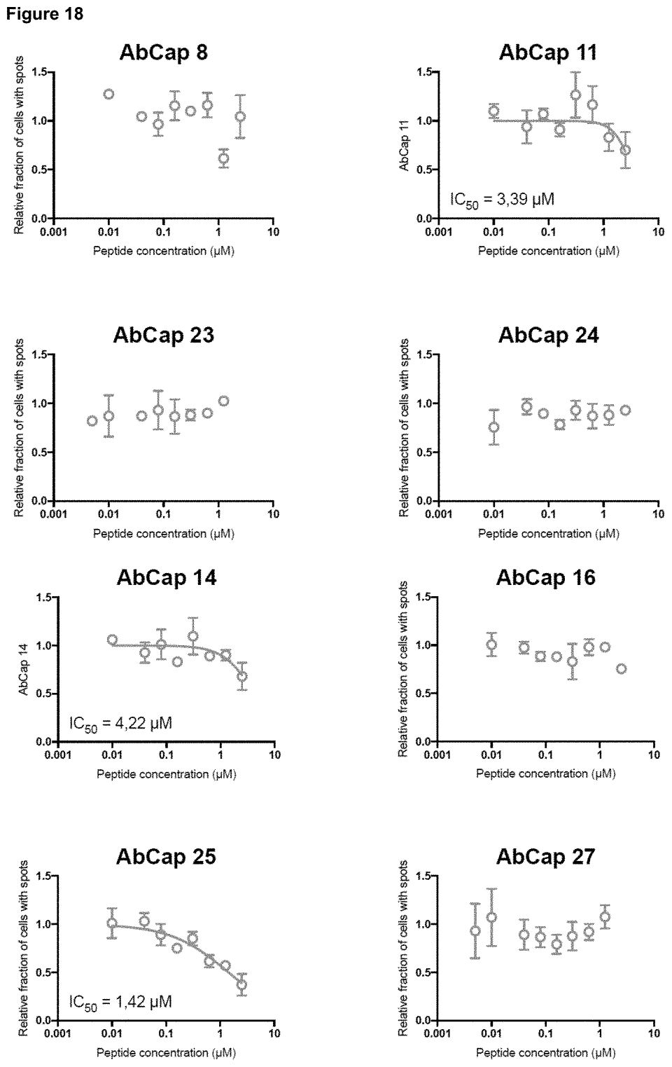

Instead of showing all the representative images, FIG. 18 shows the relative number of cells with spots.

In other words, the condition in which no seeds are added is shown as 0, while the condition in which 100 nM Aβ aggregates are added (without capping peptides) is shown as 1.

FIG. 18 shows that hetero-tandem capping peptide AbCap 25 has a concentration dependent effect on the aggregation seeding capacity of Aβ, with an IC₅₀ of 1,42 μM. Noteworthy, hetero-tandem capping peptides AbCap 11 and AbCap 14 also have an effect at the highest concentrations.

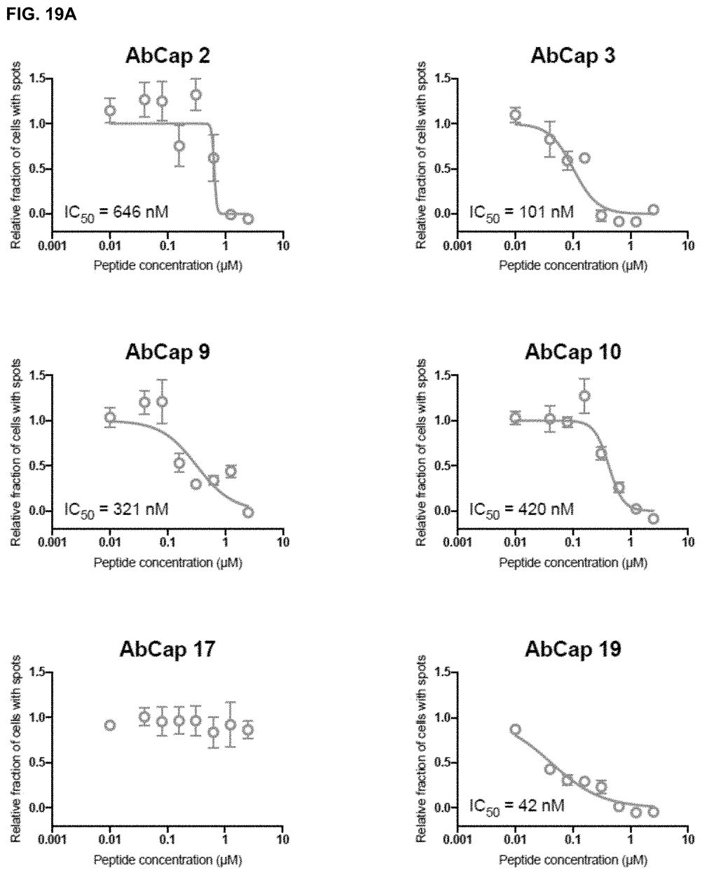

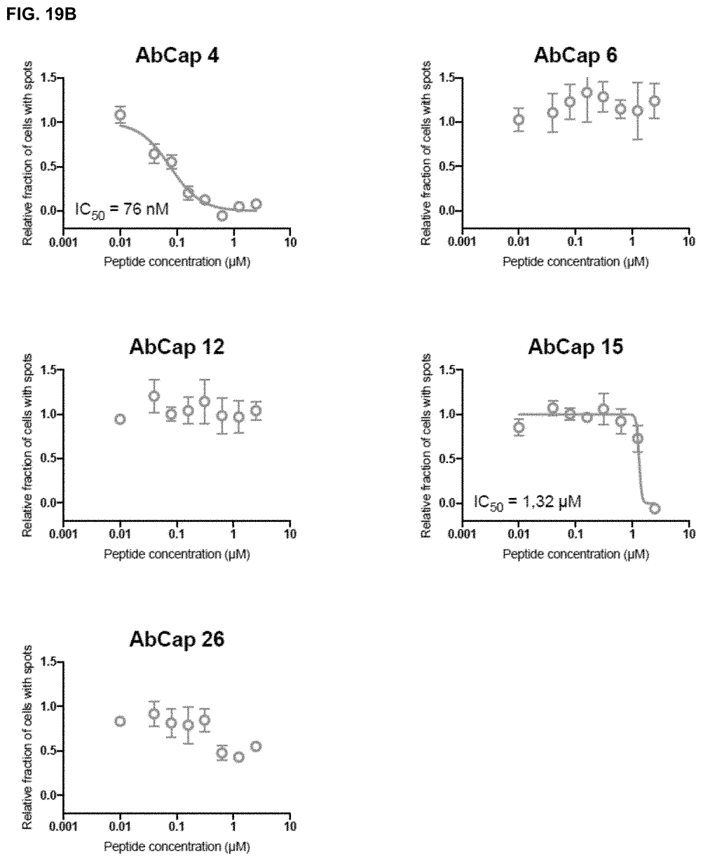

In a next step a second batch of hetero-tandem capping peptides was screened and the data are depicted in FIG. 19.

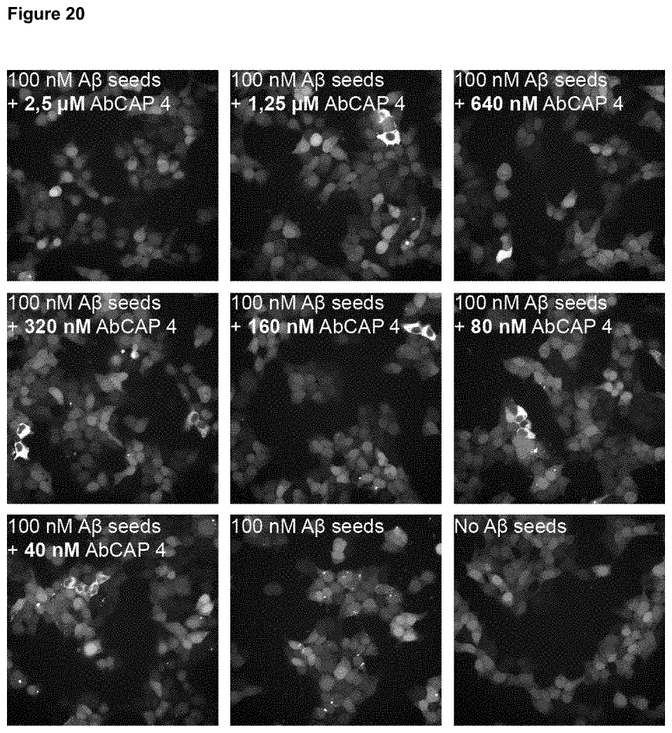

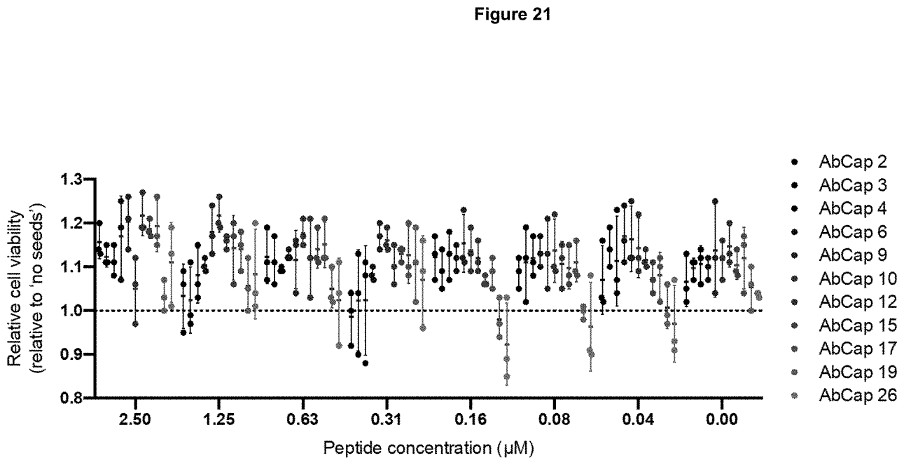

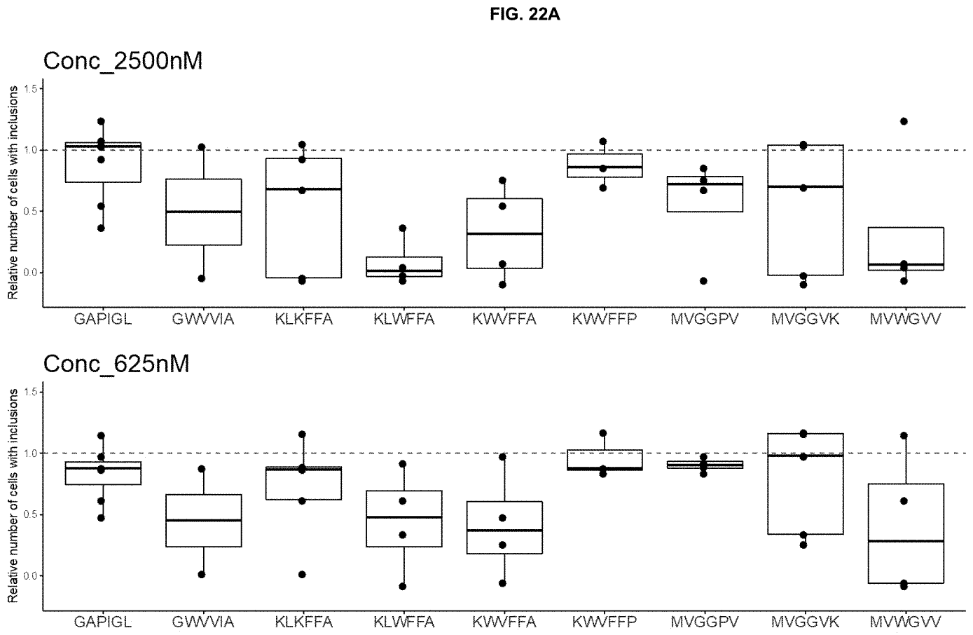

An example of the effect of one hetero-tandem capping (AbCap 4) is shown in FIG. 20. Additionally, FIG. 21 shows the relative cell viability of all treated cells. Data is normalized to non-treated cells (where no seeds were added).

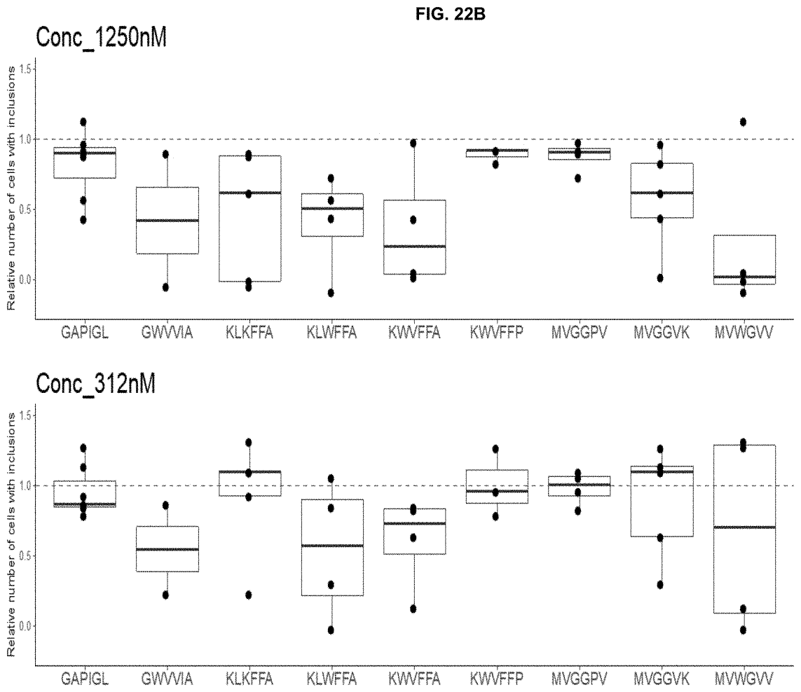

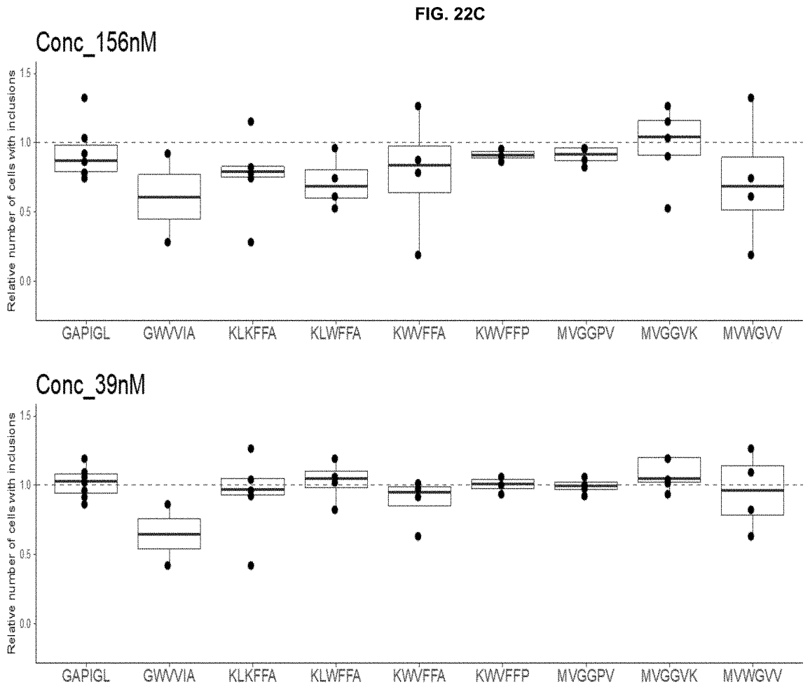

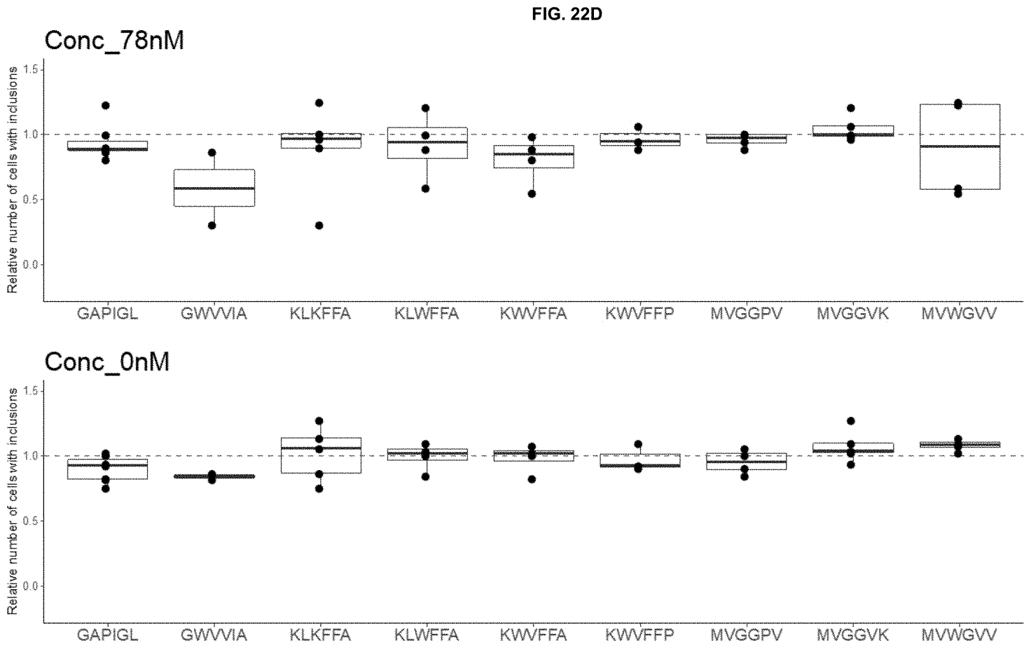

In addition, we performed an analysis on the activity of all tandem capping peptides to check whether a specific single capping peptide in all the tandems consistently shows an increased capping activity. This analysis is shown in FIG. 22.

From this experiment it is noted that two single capping peptides score well at the highest concentrations: KLWFFA (SEQ ID NO: 90) and MVWGVV (SEQ ID NO: 91).

12.2 Aβ Biosensor Assay—Aβ-647 Seeds

Based on the results presented in FIGS. 18, 19 and 20 we selected the most promising capping peptide hits. The sequences of these hits are depicted in Table 6.

TABLE 6

| ID | SEQUENCE |
|---|---|
| AbCap_2 | MVWGVVGSKLKFFA (SEQ ID NO: 64) |
| AbCap_3 | MVWGVVGSKLWFFA (SEQ ID NO: 65) |
| AbCap_4 | MVWGVVGSKWVFFA (SEQ ID NO: 66) |
| AbCap_8 | MVGGVKGSKLKFFA (SEQ ID NO: 70) |
| AbCap_9 | MVGGVKGSKLWFFA (SEQ ID NO: 71) |
| AbCap_10 | MVGGVKGSKWVFFA (SEQ ID NO: 72) |
| AbCap_15 | MVGGPVGSKLWFFA (SEQ ID NO: 77) |
| AbCap_19 | GWVVIAGSKLKFFA (SEQ ID NO: 81) |
| AbCap_25 | KLWFFAGSGAPIGL (SEQ ID NO: 87) |

sequences of most optimal capping peptides identified in section 12.1

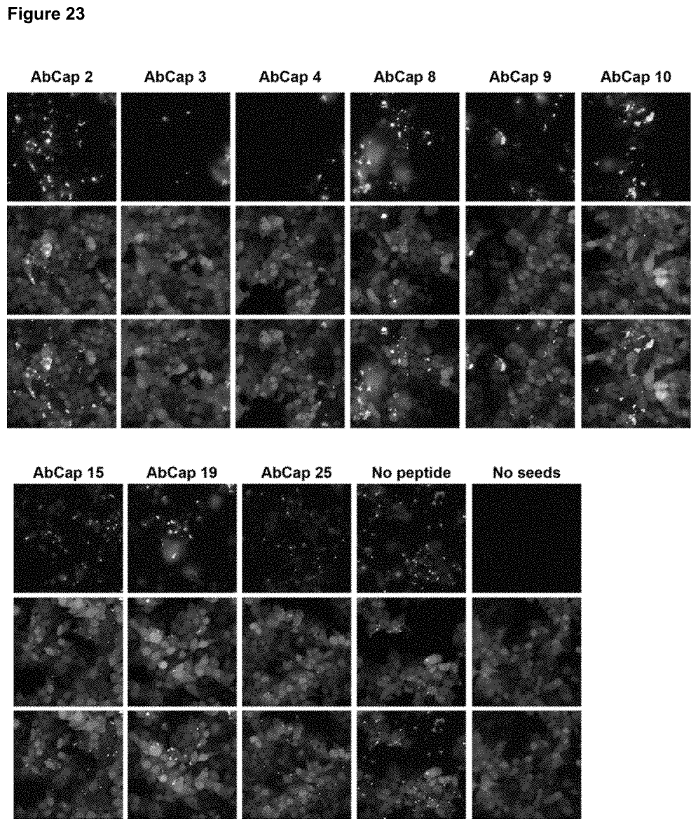

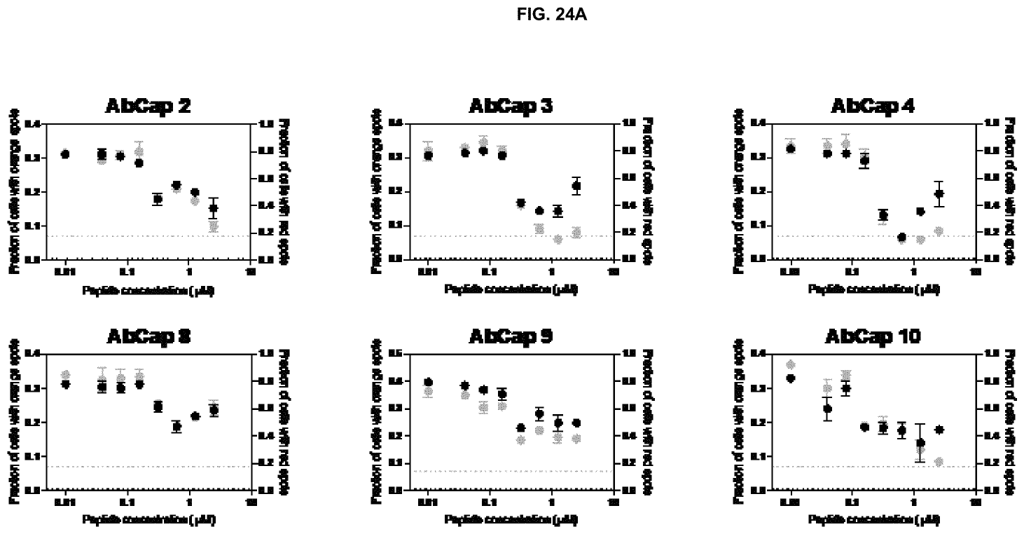

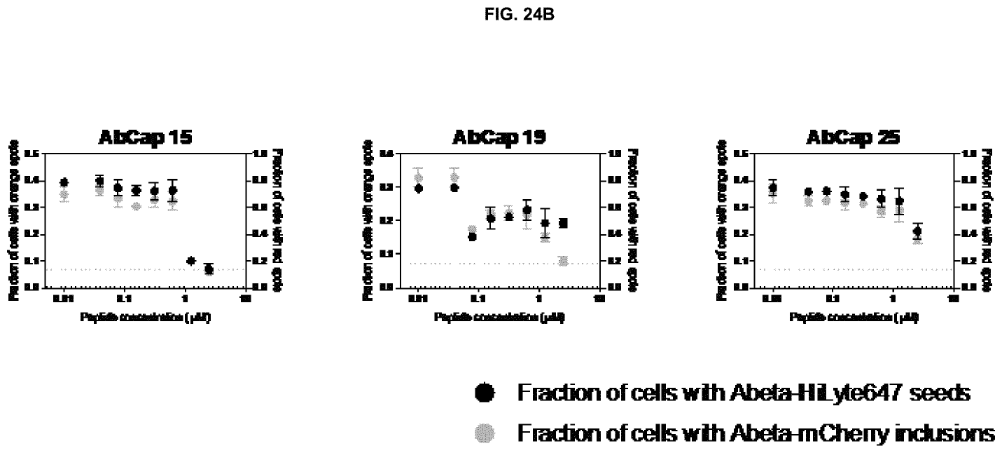

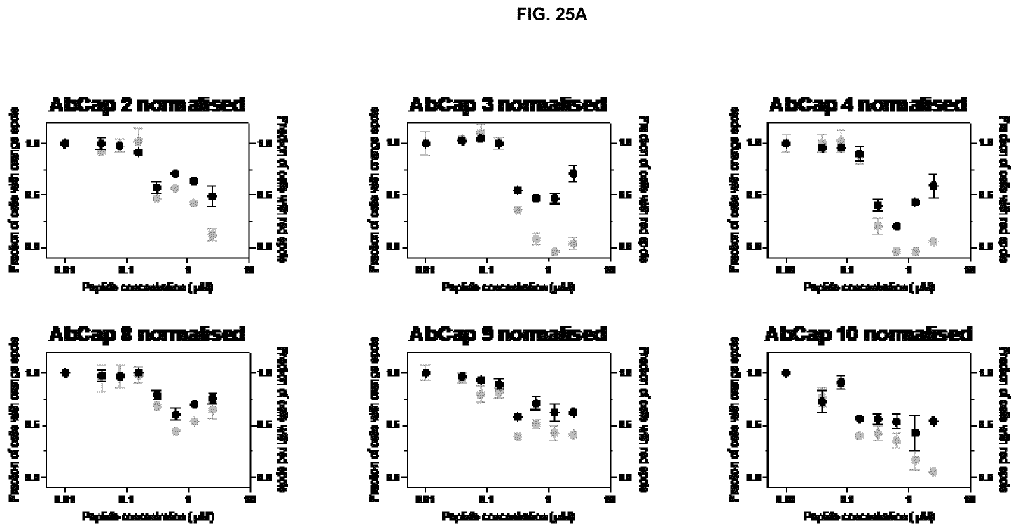

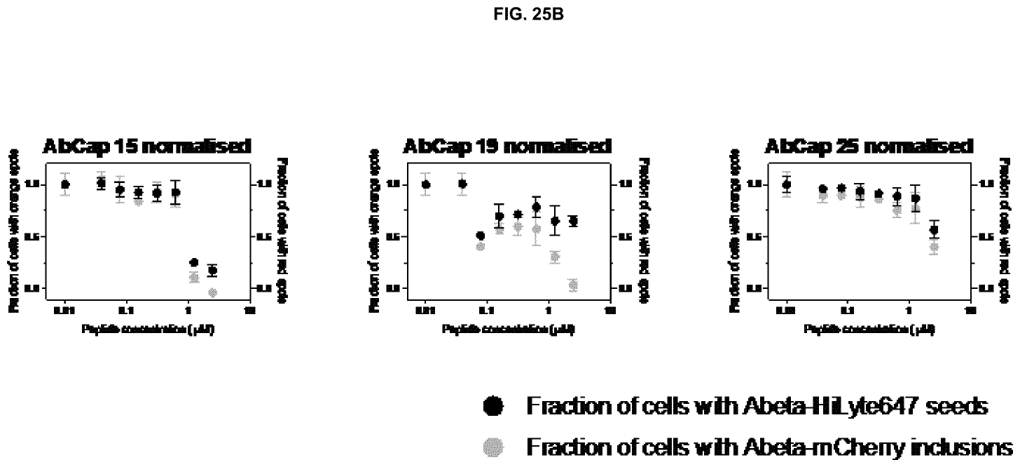

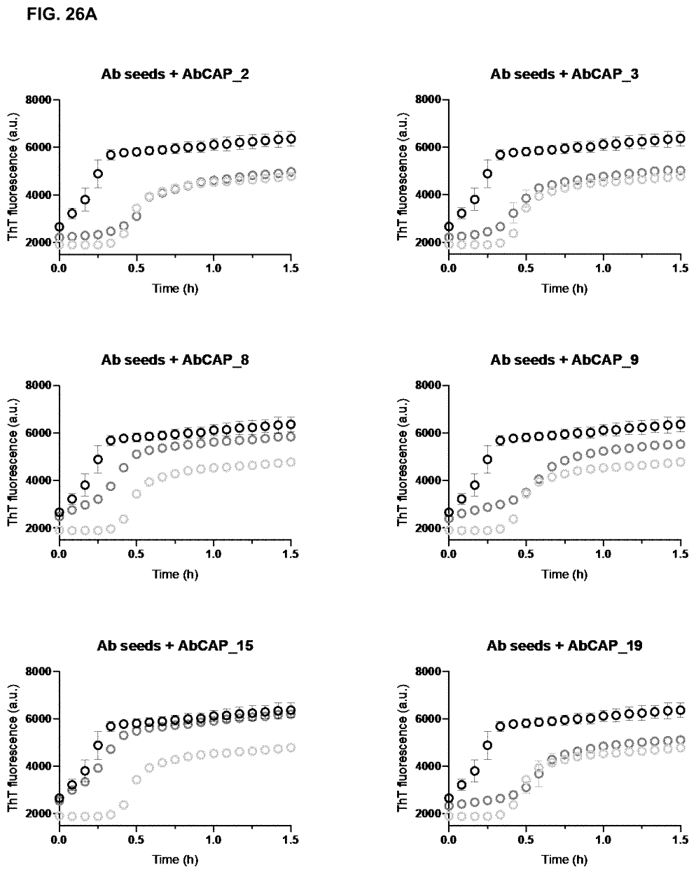

Hetero-tandem capping peptides from Table 6 were dissolved in DMSO, filtered and mixed with preformed, sonicated Aβ-647 aggregates (10 μM). This mixture was incubated for approx. 16 hours and transfected into Aβ biosensor cells. The final concentration of Aβ-647 aggregates on the cells is 500 nM and a concentration range of capping peptides was used. Data are shown in FIGS. 23, 24 and 25.

12.3 In Vitro Aβ Seeding Aggregation Assay

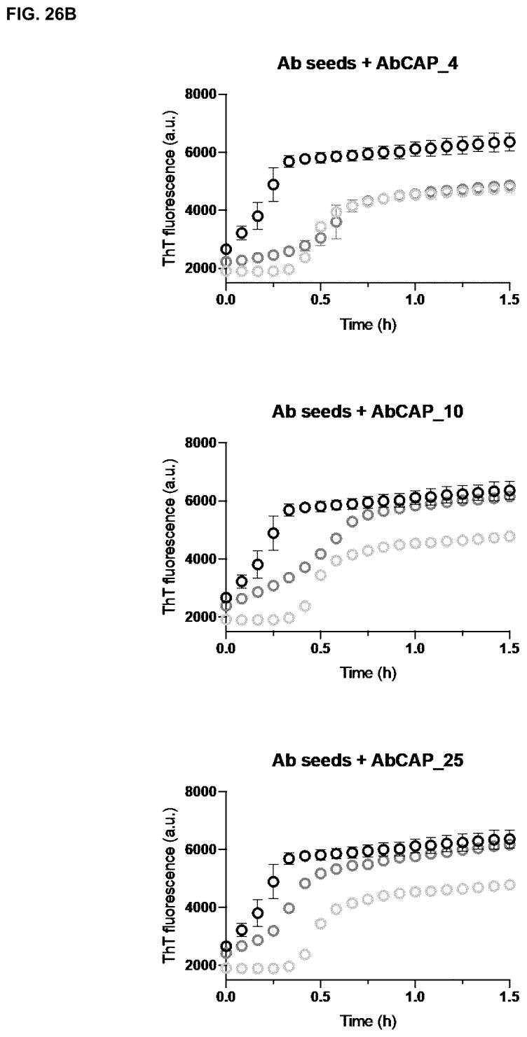

Capping peptides from Table 6 were freshly dissolved in DMSO (stock concentration 1 mM) and filtered before the assay. 1 μL peptide capping solution was mixed with 19 μL preformed, sonicated Aβ fibrils (stock concentration 10 μM). As such, this mixture contained 10 μM Aβ fibrils and 50 μM capping peptide and was incubated for approx. 4 hours. Next, freshly dissolved monomeric Aβ was prepared by separation on a column. This monomeric Aβ (final concentration 10 μM) was mixed with 10% of the Aβ fibrils-peptide mixture and Th-T was added to monitor aggregation. Results of the data are shown in FIG. 26.

We conclude that capping peptides AbCAP_2, AbCAP3, AbCAP4 and AbCAP19 have a clear effect on the seeding capacity of preformed, sonicated Aβ fibrils.

12.4 In Vitro Affinity Assay

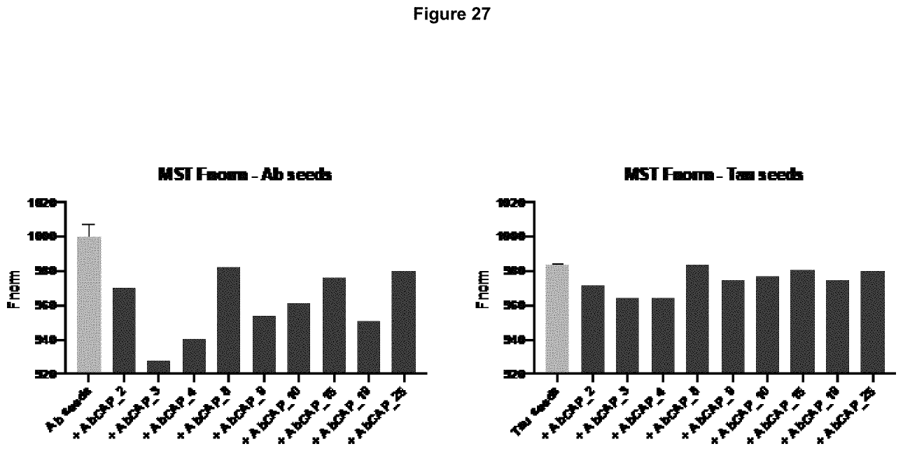

Capping peptides from Table 6 were freshly dissolved in DMSO (stock concentration 1 mM) and filtered before the assay. 1 μL peptide capping solution was mixed with 19 μL preformed, sonicated Aβ-647 fibrils (stock concentration 500 nM) or sonicated Tau-633 fibrils (250 nM). MST measurements of this mixture were immediately monitored. FIG. 27 represents Fnorm values; in short: a difference in Fnorm value between the sonicated fibrils alone and the sonicated fibrils mixed with capping peptide indicates a binding event.

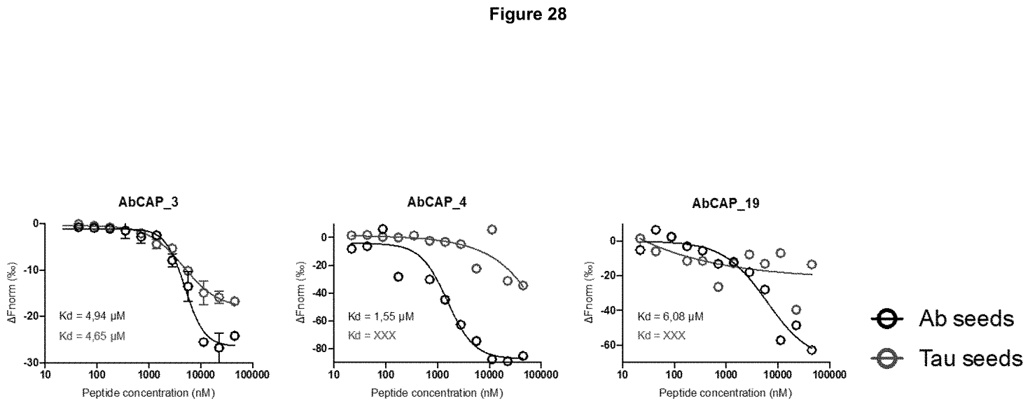

For the three most promising capping peptides (AbCap3, 4 and 19), we performed the same experiment as described above with a concentration range of capping peptide against both sonicated Aβ-647 fibrils and sonicated tau-633 fibrils. Data are shown in FIG. 28.

13. Design of Capping Peptides to Additional Proteins which can form Pathological Aggregates

13.1 Capping Peptides Directed to Pathological Aggregates of Transthyretin

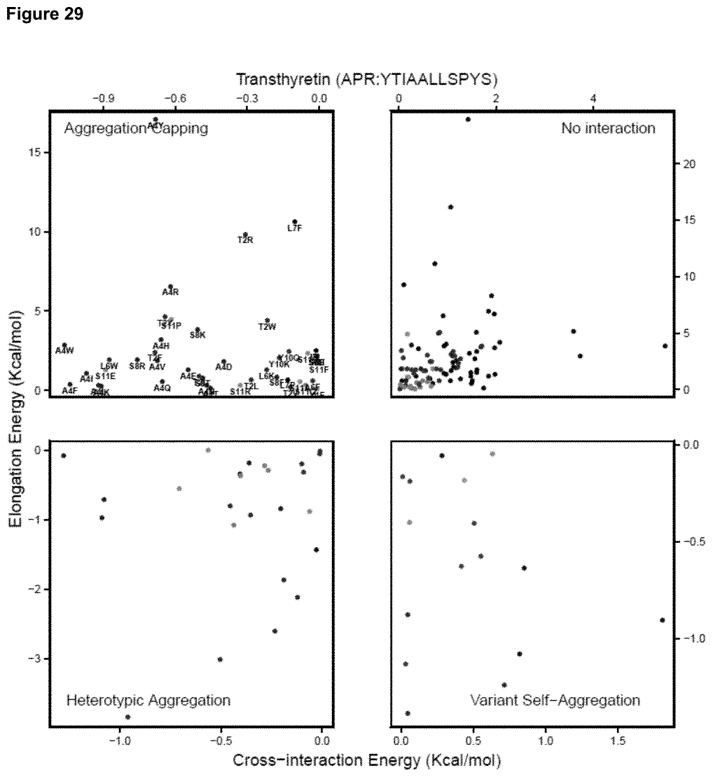

Transthyretin (TTR) is a protein which can form pathological aggregates. Pathological aggregates of TTR are associated with senile systemic amyloidosis, familial amyloidotic polyneuropathy, familial amyloid cardiomyopathy and leptomeningeal amyloidosis (Chiti F and Dobson C M (2017) Annu. Rev. Biochem. 86: 27-68). Capping peptides were designed targeting pathological aggregates of TTR as described in example 1. The amino acid sequence of TTR is available as UniProt accession number (https://www.uniprot.org) P02766. An amyloid core structure for the target APR sequence YTIAALLSPYS (SEQ ID NO: 92) present in TTR is available in the protein structure database (www.rcsb.org) as 2m5n. The analysis based on this amyloid core structure conducted as outlined in example 1 is shown in FIG. 29.

Three predicted capping peptides identified from the analysis are YTIYALLSPYS (SEQ ID NO: 93), YTIAPLLSPYS (SEQ ID NO: 94) and YTIAALFSPYS ((SEQ ID NO: 95).

Thus, non-natural molecules comprising capping peptides targeting a pathological aggregate of TTR linked to a moiety targeting the intracellular proteolysis system are provided for use to treat senile systemic amyloidosis, familial amyloidotic polyneuropathy, familial amyloid cardiomyopathy and leptomeningeal amyloidosis.

13.2 Capping Peptides Directed to Pathological Aggregates of Insulin

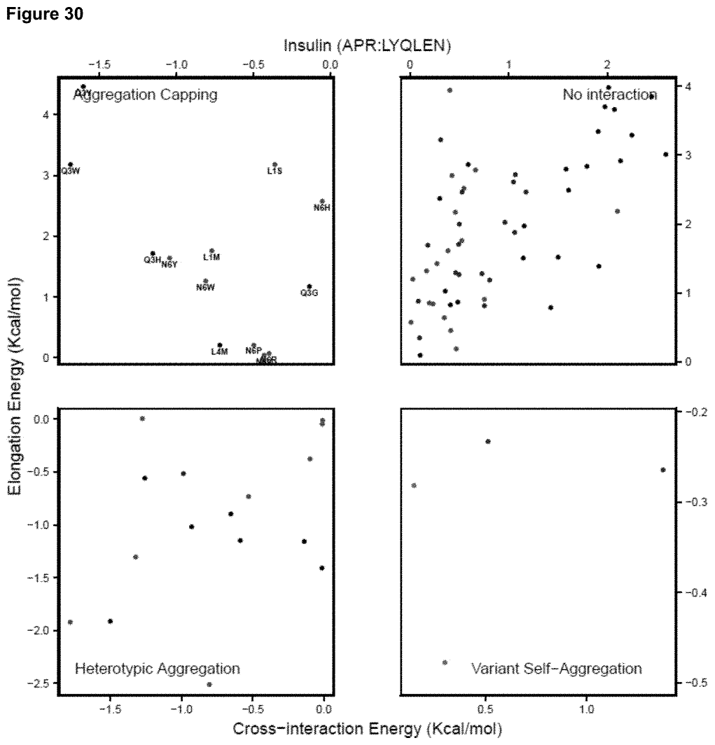

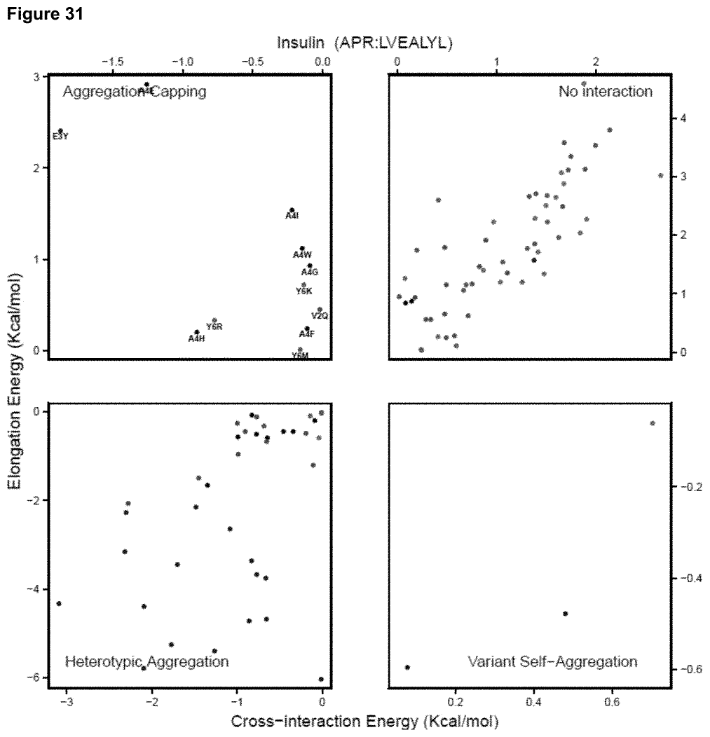

Insulin is a polypeptide which can form pathological aggregates. Pathological aggregates of insulin are associated with injection-localized amyloidosis (Chiti F and Dobson C M (2017) Annu. Rev. Biochem. 86: 27-68). Capping peptides were designed targeting pathological aggregates of insulin as described in example 1. The amino acid sequence of insulin is available as UniProt accession number (https://www.uniprot.org) A6XGL2. Two amyloid core structures for the target APR sequences LYQLEN (SEQ ID NO: 96) and LVEALYL (SEQ ID NO: 97) present in insulin is available in the protein structure database (www.rcsb.org) respectively as 2omp and 3hyd. The analysis based on these amyloid core structures were conducted as outlined in example 1 and are shown in FIGS. 30 and 31.

Three predicted capping peptides identified from the analysis on the core structure 2omp are LYYLEN (SEQ ID NO: 98), LYWLEN (SEQ ID NO: 99) and SYQLEN (SEQ ID NO: 100).

Three predicted capping peptides identified from the analysis on the core structure 3hyd are LVEASYL (SEQ ID NO: 101), LVYALYL (SEQ ID NO: 102) and LVEALYL (SEQ ID NO: 103).

Thus, non-natural molecules comprising capping peptides targeting pathological aggregates of insulin linked to a moiety targeting the intracellular proteolysis system are provided for use to treat injection-localized amyloidosis.

13.3 Capping Peptides Directed to Pathological Aggregates of Islet Amyloid Polypeptide (IAPP)

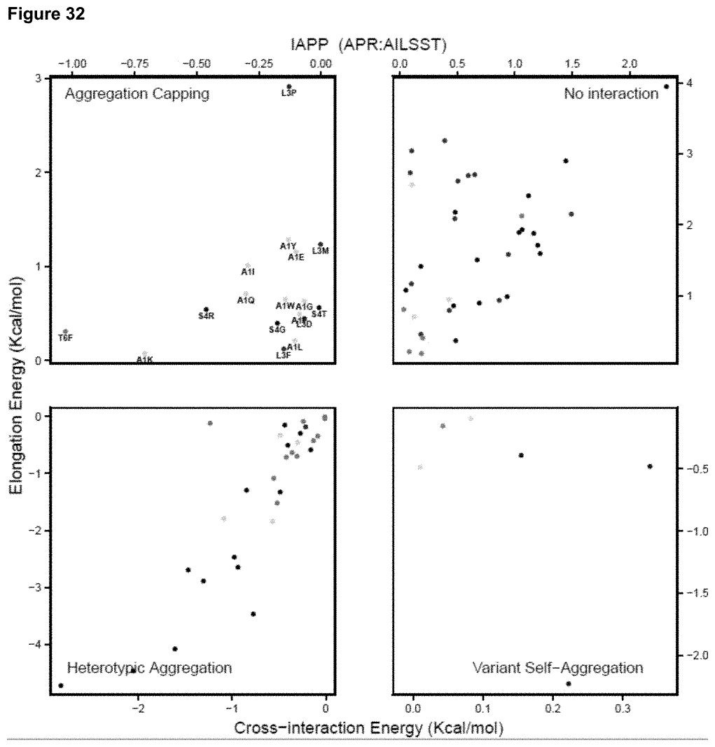

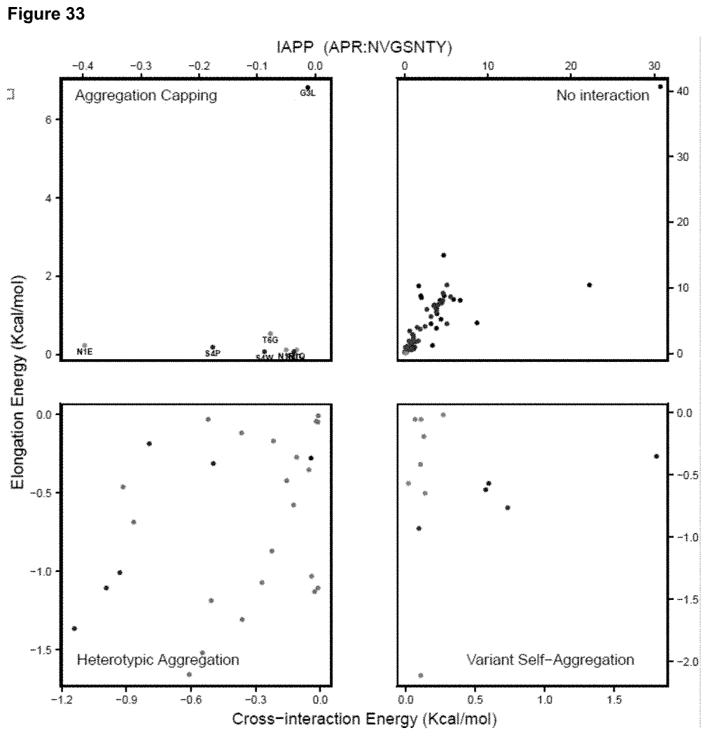

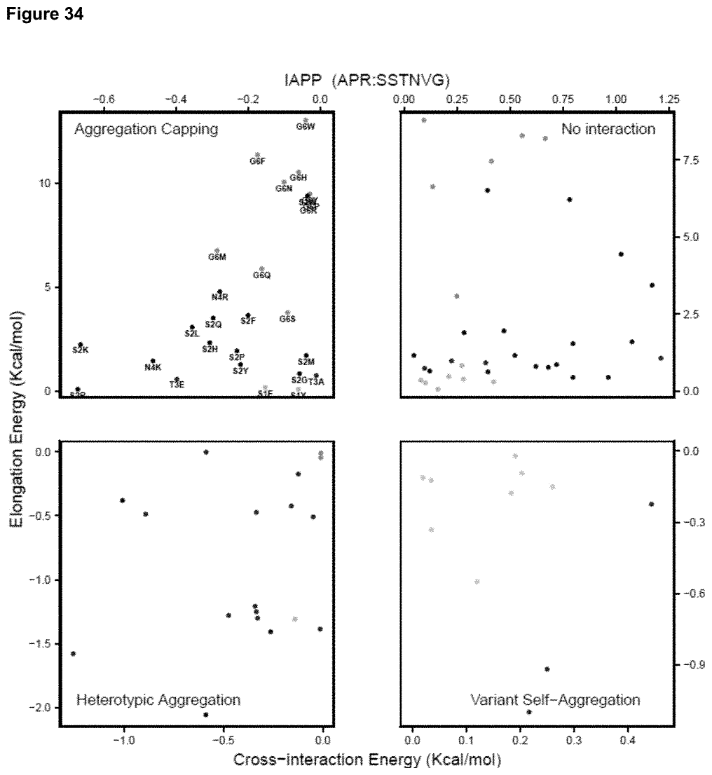

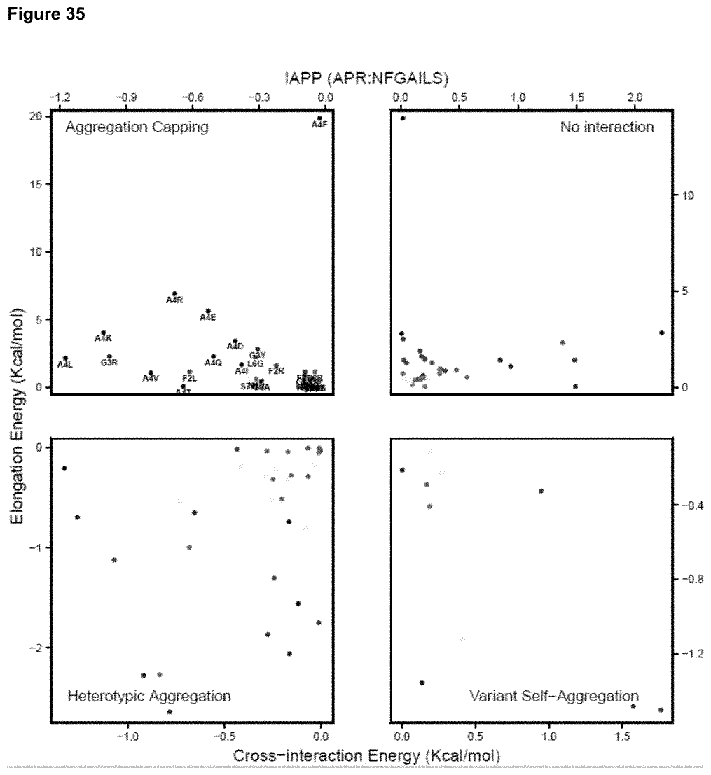

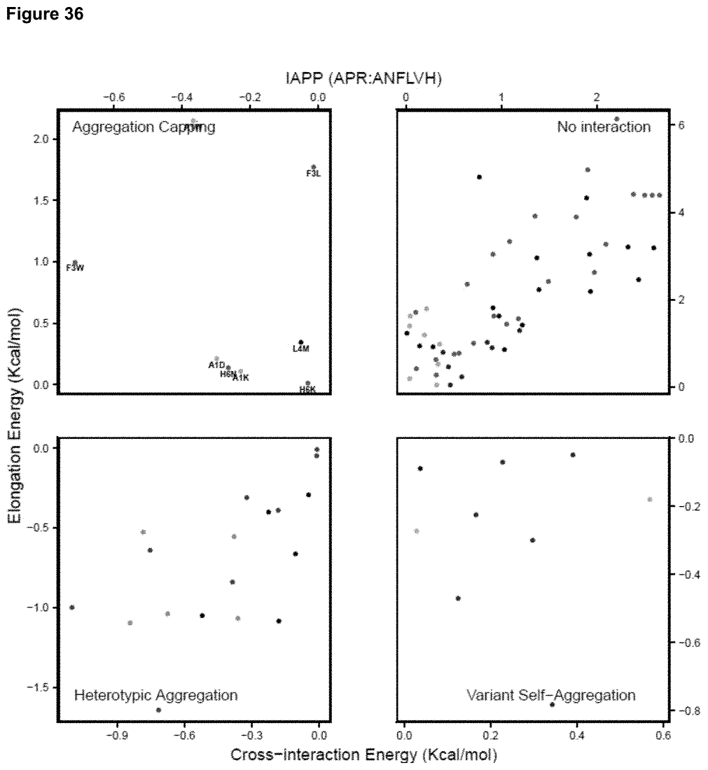

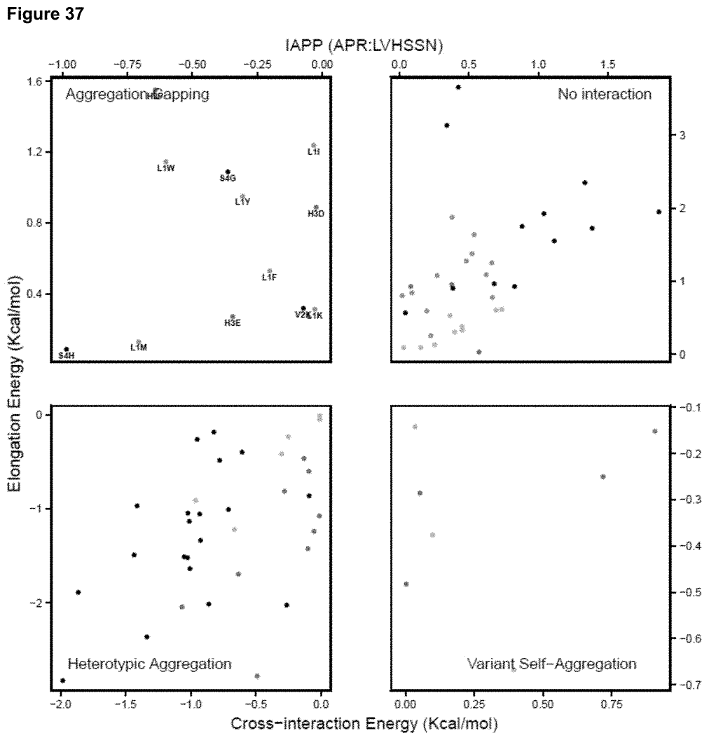

Islet amyloid polypeptide (IAPP) is a polypeptide which can form pathological aggregates. Pathological aggregates of IAPP are associated with type II diabetes and with insulinoma (Chiti F and Dobson CM (2017) Annu. Rev. Biochem. 86: 27-68). Capping peptides were designed targeting pathological aggregates of IAPP as described in example 1. The amino acid sequence of IAPP is available as UniProt accession number (https://www.uniprot.org) P10997. Several amyloid core structures for different APR sequences present in IAPP is available in the protein structure database (www.rcsb.org) as 3fod (for AILSST (SEQ ID NO: 104), 3 ftl (for NVGSNTY (SEQ ID NO: 105), 3ftr (for SSTNVG (SEQ ID NO: 106), 5e5v (for NFGAILS (SEQ ID NO: 107), 5e5x (for ANFLVH (SEQ ID NO: 108) and 5e5z (for LVHSSN (SEQ ID NO: 109). The analysis based on these amyloid core structures are conducted as outlined in example 1 and are shown in FIGS. 32 to 37.

Three predicted capping peptides identified from the analysis based on the amyloid core 3fod are AIPSST (SEQ ID NO: 110), AILSSF (SEQ ID NO: 111) and AILSPT (SEQ ID NO: 112).

Three predicted capping peptides identified from the analysis based on the amyloid core 3ft1 are NVGSLTY (SEQ ID NO: 113), EVGSNTY (SEQ ID NO: 114) and NVGSNGY (SEQ ID NO: 115).

Three predicted capping peptides identified from the analysis based on the amyloid core 3ftr are SKTNVG (SEQ ID NO: 116), SSTNVE (SEQ ID NO: 117) and SSTNVW (SEQ ID NO: 118).

Three predicted capping peptides identified from the analysis based on the amyloid core 5e5v are NFGFILS (SEQ ID NO: 119), NFGRILS (SEQ ID NO: 120) and NFGEILS (SEQ ID NO: 121).

Three predicted capping peptides identified from the analysis based on the amyloid core 5e5x are WNFLVH (SEQ ID NO: 122), ANWLVH (SEQ ID NO: 123) and ANFLRH (SEQ ID NO: 124).

Three predicted capping peptides identified from the analysis based on the amyloid core 5e5z are LVPSSN (SEQ ID NO: 125), WVHSSN (SEQ ID NO: 126) and LVHGSN (SEQ ID NO: 127).

Thus, non-natural molecules comprising capping peptides targeting a pathological aggregate of IAPP linked to a moiety targeting the intracellular proteolysis system are provided for use to treat diabetes type II and insulinoma.

13.4 Capping Peptides Directed to Pathological Aggregates of Beta-2-Microglobulin

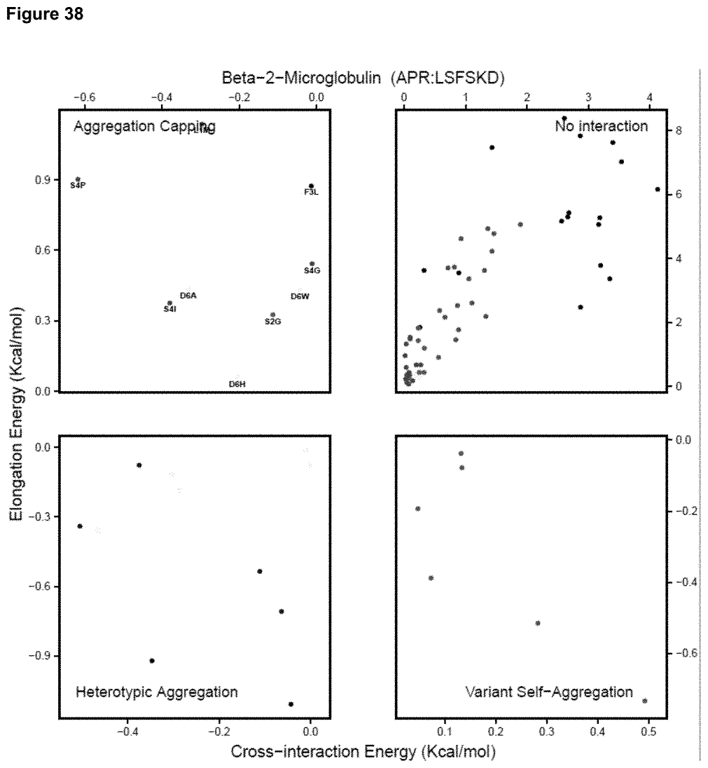

Beta2-microglobulin is a protein which can form pathological aggregates. Pathological aggregates of beta2-microglobulin are associated with dialysis-related amyloidosis and hereditary visceral amyloidosis (Chiti F and Dobson C M (2017) Annu. Rev. Biochem. 86: 27-68). Capping peptides were designed targeting a pathological aggregate of beta2-microglobulin as described in example 1. The amino acid sequence of beta2-microglobulin is available as UniProt accession number (https://www.uniprot.org) P61769. An amyloid core structure for the target APR sequence LSFSKD (SEQ ID NO: 128) present in beta2-microglobulin is available in the protein structure database (www.rcsb.org) as 31oz. The analysis based on this amyloid core structure conducted as outlined in example 1 is shown in FIG. 38.

Three predicted capping peptides identified from the analysis are LSFPKD (SEQ ID NO: 129), MSFSKD (SEQ ID NO: 130) and LSFSED (SEQ ID NO: 131).

Thus, non-natural molecules comprising capping peptides targeting pathological aggregates of beta2-microglobulin linked to a moiety targeting the intracellular proteolysis system are provided for use to treat dialysis-related amyloidosis and hereditary visceral amyloidosis.

13.5 Capping Peptides Directed to Pathological Aggregates of Prostatic Acid Phosphatase

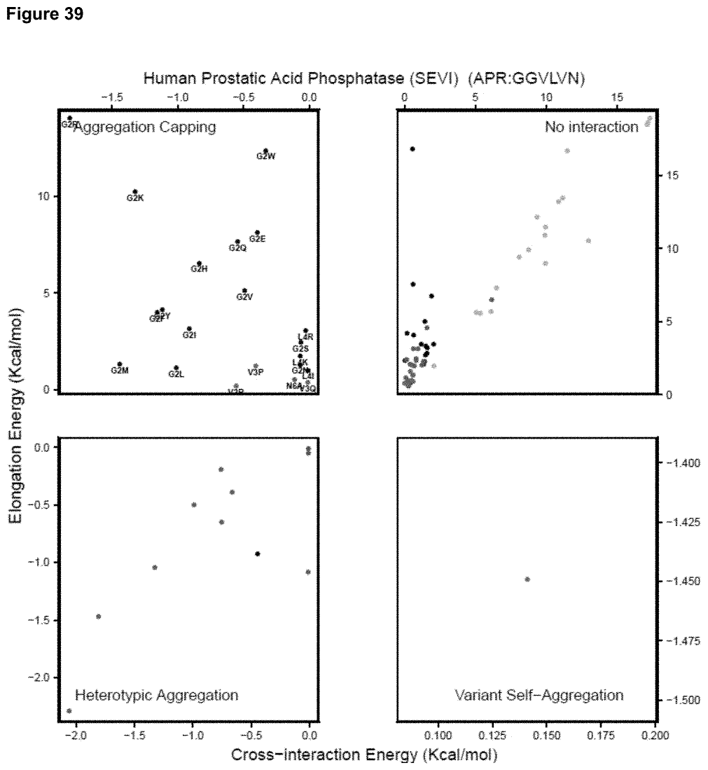

Prostatic acid phosphatase (PAP) is a protein of which fragments thereof can form pathological aggregates. Pathological aggregates of PAP are associated with enhanced HIV infection (Arnold F et al (2012) J. Virol. 86(2):1244-1249). Capping peptides were designed targeting a pathological aggregate of PAP as described in example 1. The amino acid sequence of PAP is available as UniProt accession number (https://www.uniprot.org) P15309. An amyloid core structure for the target APR sequence GGVLVN (SEQ ID NO: 132) present in PAP is available in the protein structure database (www.rcsb.org) as 3ppd. The analysis based on this amyloid core structure conducted as outlined in example 1 is shown in FIG. 39.

Three predicted capping peptides identified from the analysis are GRVLVN (SEQ ID NO: 133), GWVLVN (SEQ ID NO: 134) and GKVLVN (SEQ ID NO: 135).

Thus, non-natural molecules comprising capping peptides targeting pathological aggregates of PAP linked to a moiety targeting the intracellular proteolysis system are provided for use to treat HIV disease.

13.6 Capping Peptides Directed to Pathological Aggregates of Superoxide Dismutase 1

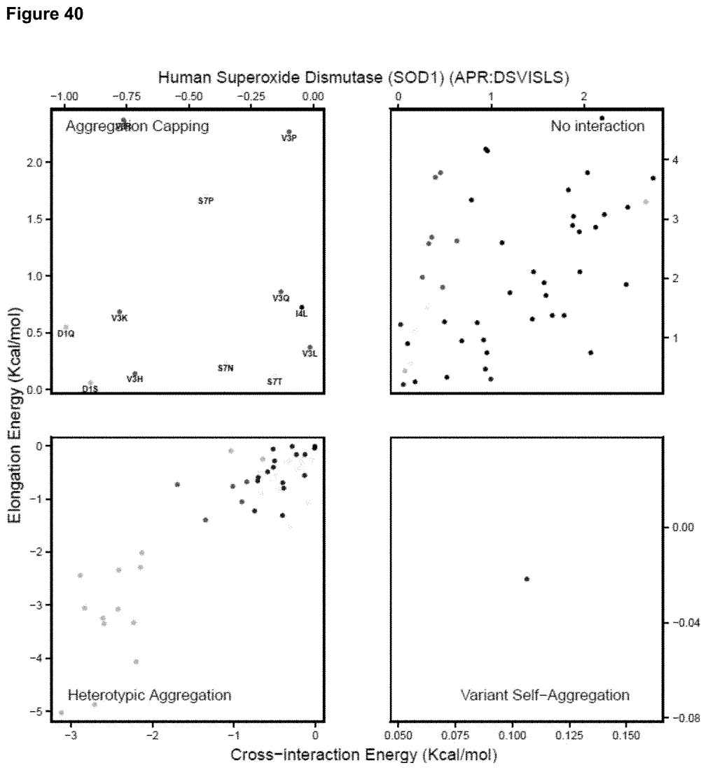

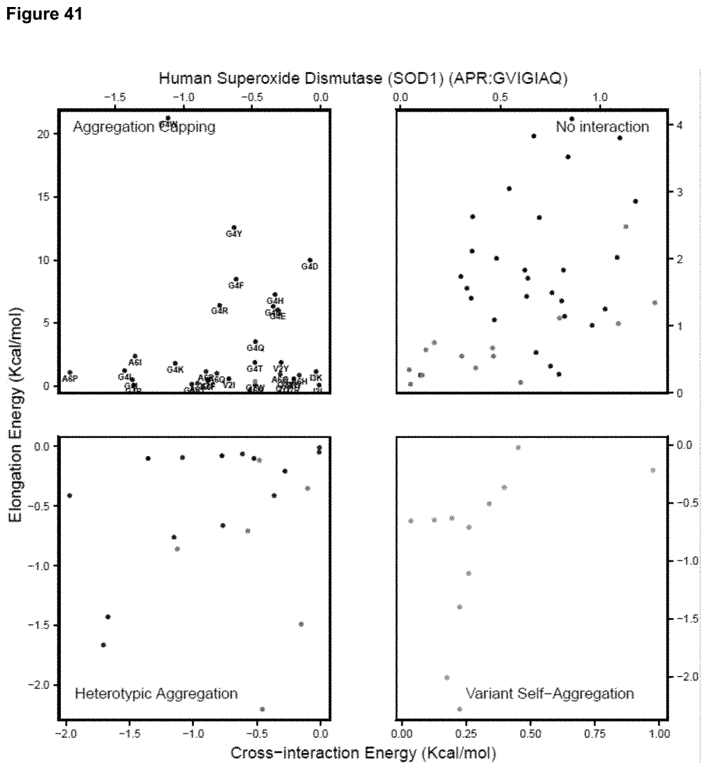

Superoxide dismutase 1 (SOD1) is a protein which can form pathological aggregates. Pathological aggregates of SOD1 are associated with Amyotrophic Lateral sclerosis (ALS) (Chiti F and Dobson C M (2017) Annu. Rev. Biochem. 86: 27-68). Capping peptides were designed targeting a pathological aggregate of SOD1 as described in example 1. The amino acid sequence of SOD1 is available as UniProt accession number (https://www.uniprot.org) P00441. Amyloid core structures for the target APR sequences DSVISLS (SEQ ID NO: 136) and GVIGIAQ (SEQ ID NO: 137) present in SOD1 is available in the protein structure database (www.rcsb.org) respectively as 4nin and 4nip. The analysis based on this amyloid core structure conducted as outlined in example 1 is shown in FIGS. 40 and 41.

Three predicted capping peptides identified from the analysis based on the amyloid core 4nin are DSRISLS (SEQ ID NO: 138), DSVISLP (SEQ ID NO: 139) and QSVISLS (SEQ ID NO: 140).

Three predicted capping peptides identified from the analysis based on the amyloid core 4nip are GVIWIAQ (SEQ ID NO: 141), GVIGIPQ (SEQ ID NO: 142) and GVIYIAQ (SEQ ID NO: 143).

Thus, non-natural molecules comprising capping peptides targeting pathological aggregates of SOD1 linked to a moiety targeting the intracellular proteolysis system are provided for use to ALS.

13.7 Capping Peptides Directed to Pathological Aggregates of Lysozyme

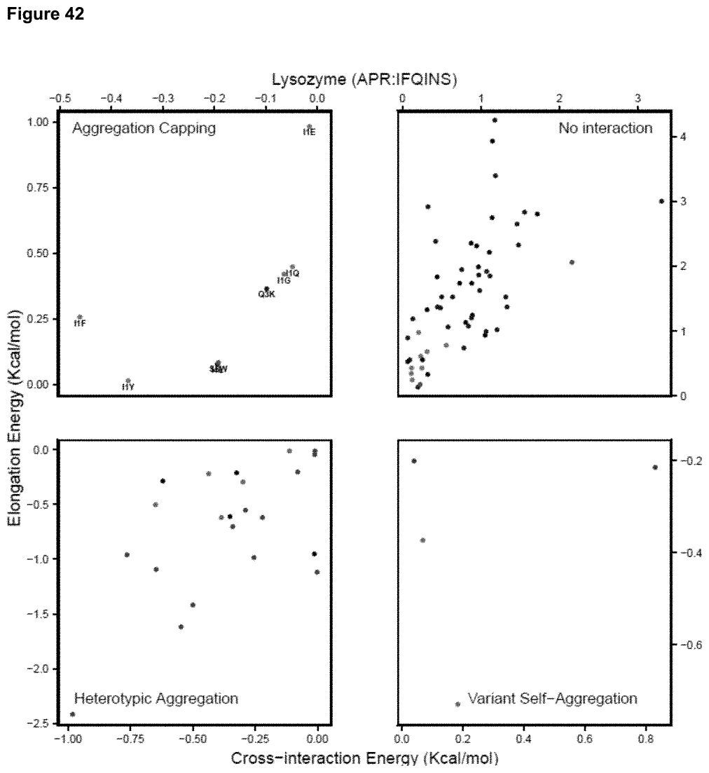

Lysozyme is a protein which can form pathological aggregates. Pathological aggregates of lysozyme are associated with lysozyme amyloidosis (mainly visceral) (Chiti F and Dobson C M (2017) Annu. Rev. Biochem. 86: 27-68). Capping peptides were designed targeting a pathological aggregate of lysozyme as described in example 1. The amino acid sequence of lysozyme is available as UniProt accession number (https://www.uniprot.org) H0YDZ_{2.} An amyloid core structure for the target APR sequence IFQINS (SEQ ID NO: 144) present in lysozyme is available in the protein structure database (www.rcsb.org) as 4r0p. The analysis based on this amyloid core structure conducted as outlined in example 1 is shown in FIG. 42.

Three predicted capping peptides identified from the analysis are IFQIES (SEQ ID NO: 145), IFQIDS (SEQ ID NO: 146) and IFQIFS (SEQ ID NO: 147).

Thus, non-natural molecules comprising capping peptides targeting pathological aggregates of lysozyme linked to a moiety targeting the intracellular proteolysis system are provided for use to treat lysozyme amyloidosis.

13.8 Capping Peptides Directed to Pathological Aggregates of Alpha-Synuclein

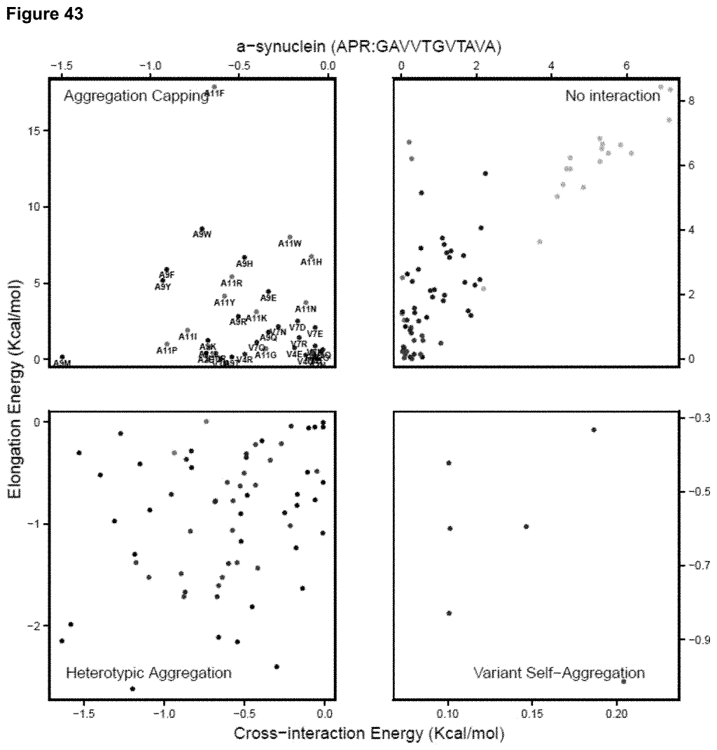

Alpha-synuclein is a polypeptide which can form pathological aggregates. Pathological aggregates of alpha-synuclein are associated with Parkinson's disease, Parkinson's disease with dementia, dementia with Lewy bodies and multiple system atrophy (Chiti F and Dobson C M (2017) Annu. Rev. Biochem. 86: 27-68). Capping peptides were designed targeting a pathological aggregate of alpha-synuclein as described in example 1. The amino acid sequence of alpha-synuclein is available as UniProt accession number (https://www.uniprot.org) P37840. An amyloid core structure for the target APR sequence GAVVTGVTAVA (SEQ ID NO: 148) present in alpha-synuclein is available in the protein structure database (www.rcsb.org) as 4ri1. The analysis based on this amyloid core structure was conducted as outlined in example 1 and is shown in FIG. 43.

Three predicted capping peptides identified from the analysis on the core structure 4ri1 are GAVVTGVTAVF (SEQ ID NO: 149), GAVVTGVTAVA (SEQ ID NO: 150) and GAVVTGVTAVA (SEQ ID NO: 151).

Thus, non-natural molecules comprising capping peptides targeting pathological aggregates of alpha-synuclein aggregates linked to a moiety targeting the intracellular proteolysis system are provided for use to treat Parkinson's disease, Parkinson's disease with dementia, dementia with Lewy bodies and multiple system atrophy.

13.9 Capping Peptides Directed to Pathological Aggregates of p53

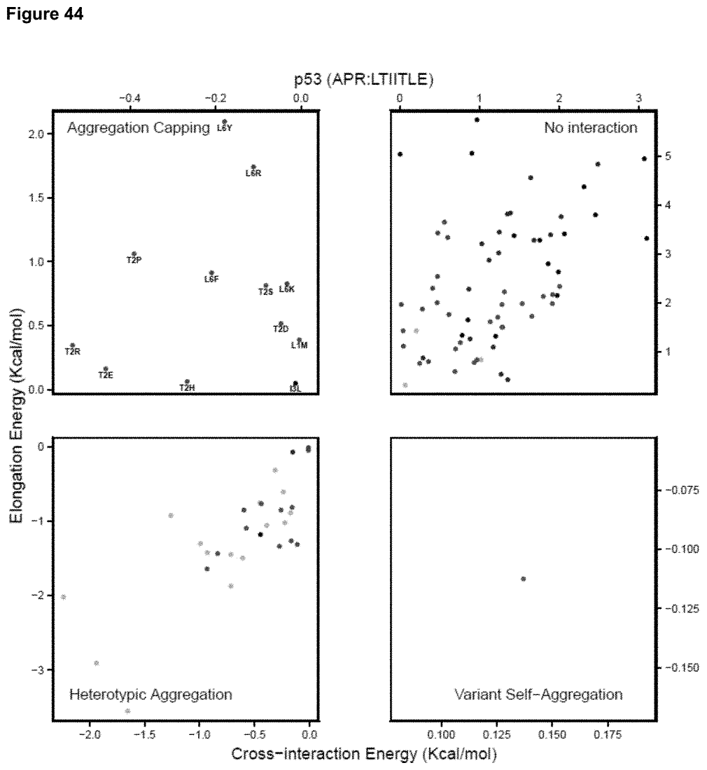

The protein p53 is a polypeptide which can form pathological aggregates. Pathological aggregates of p53 are associated with cancer (Chiti F and Dobson C M (2017) Annu. Rev. Biochem. 86: 27-68). Capping peptides were designed targeting a pathological aggregate of p53 as described in example 1. The amino acid sequence of p53 is available as UniProt accession number (https://www.uniprot.org) P04637. An amyloid core structure for the target APR sequence LTIITLE (SEQ ID NO: 152) present in P53 is available in the protein structure database (www.rcsb.org) as 4rp6. The analysis based on this amyloid core structure was conducted as outlined in example 1 and is shown in FIG. 44.

Three predicted capping peptides identified from the analysis on the core structure 4rp6 are LTIITYE (SEQ ID NO: 153), LTIITLE (SEQ ID NO: 154) and LPIITLE (SEQ ID NO: 155).

Thus, non-natural molecules comprising capping peptides targeting p53 aggregates linked to a moiety targeting the intracellular proteolysis system are provided for use to treat cancer.

13.10 Capping Peptides Directed to Pathological Aggregates of Prion Protein

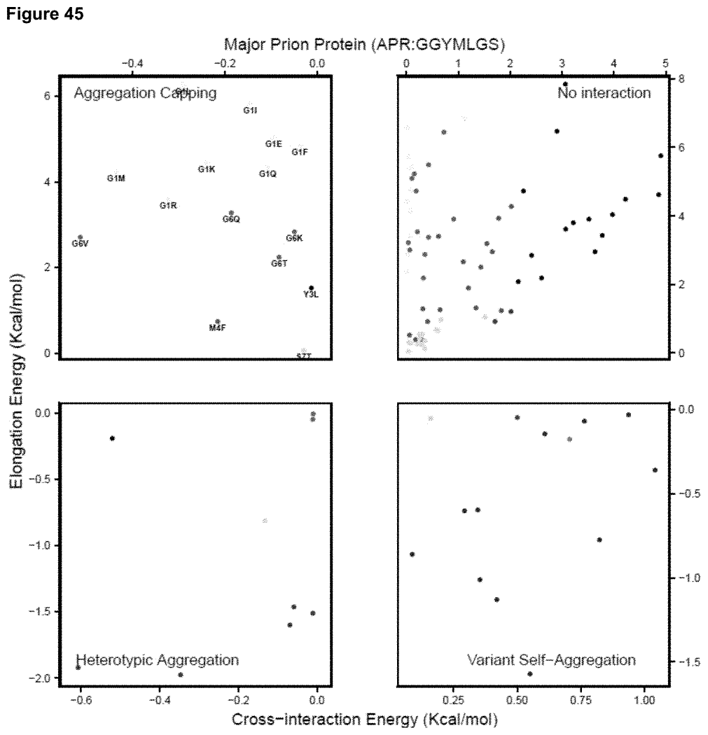

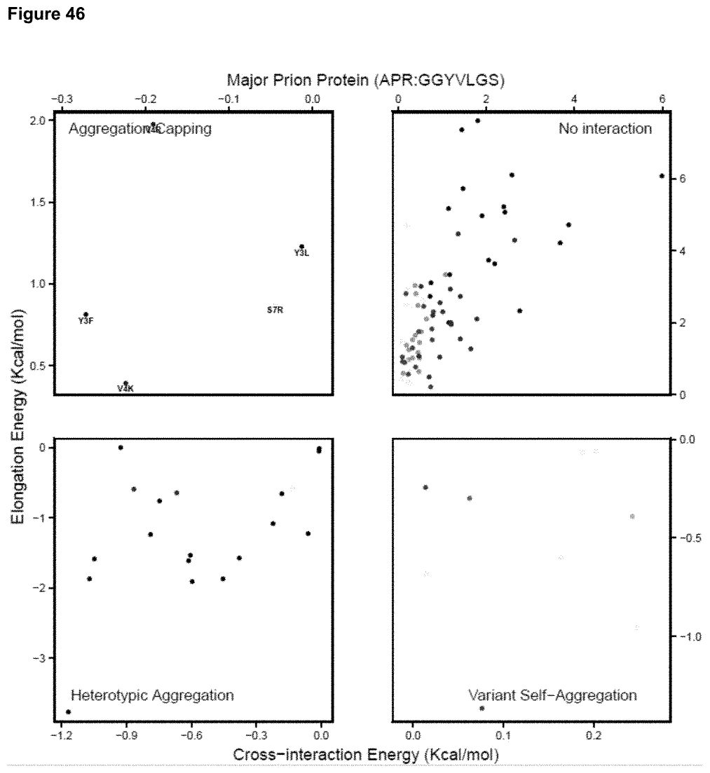

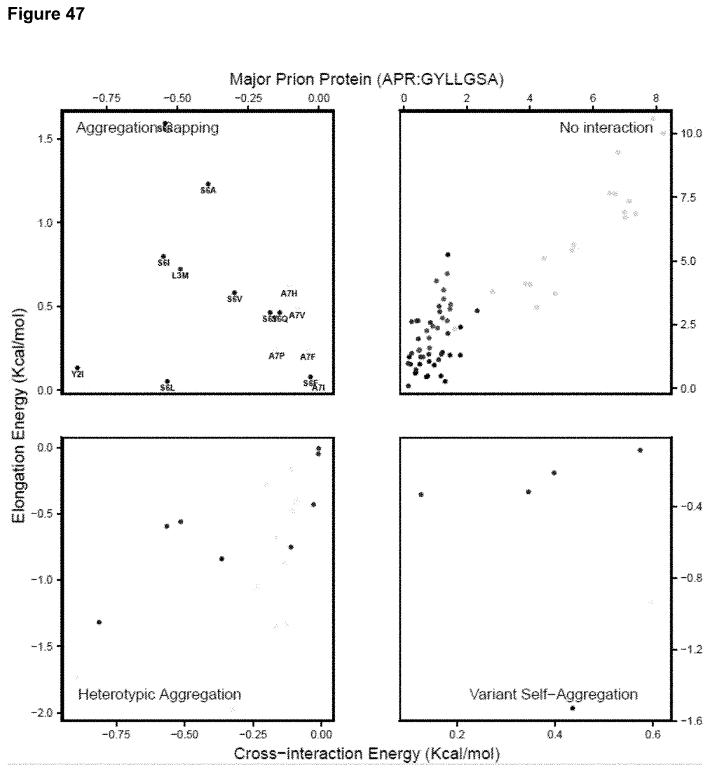

The prion protein (PrP) is a polypeptide which can form pathological aggregates. Pathological aggregates of PrP are associated with Creutzfeldt-Jacob disease, fatal insomnia, Gerstmann-Sträussler-Scheinker disease, Huntington disease-like 1, Spongiform encephalopathy with neuropsychiatric features, New variant Creutzfeldt-Jacob disease, Kuru and Hereditary sensory and autonomic neuropathy (Chiti F and Dobson C M (2017) Annu. Rev. Biochem. 86: 27-68). Capping peptides were designed targeting a pathological aggregate of PrP as described in example 1. The amino acid sequence of PrP is available as UniProt accession number (https://www.uniprot.org3 P04156. Amyloid core structures for the target APR sequences GGYMLGS (SEQ ID NO: 156), GGYVLGS (SEQ ID NO: 157) and GYLLGSA (SEQ ID NO: 158) present in PrP is available in the protein structure database (www.rcsb.org) as respectively 4w5m, 4w5p and 4w71. The analysis based on these amyloid core structures was conducted as outlined in example 1 and is shown in FIGS. 45, 46 and 47.

Three predicted capping peptides identified from the analysis on the core structure 4w5m are LGYMLGS (SEQ ID NO: 159), GGYMLVS (SEQ ID NO: 160) and KGYMLGS (SEQ ID NO: 161).

Three predicted capping peptides identified from the analysis on the core structure 4w5p are GGYVYGS (SEQ ID NO: 162), GGYRLGS (SEQ ID NO: 163) and GGYVFGS (SEQ ID NO: 164).

Three predicted capping peptides identified from the analysis on the core structure 4w71 are GYLLHSA (SEQ ID NO: 165), GYLLYSA (SEQ ID NO: 166) and GYLLFSA (SEQ ID NO: 167).

Thus, non-natural molecules comprising capping peptides targeting pathological aggregates of PrP linked to a moiety targeting the intracellular proteolysis system are provided for use to treat Creutzfeldt-Jacob disease, fatal insomnia, Gerstmann-Straussler-Scheinker disease, Huntington disease-like 1, Spongiform encephalopathy with neuropsychiatric features, New variant Creutzfeldt-Jacob disease, Kuru and Hereditary sensory and autonomic neuropathy.

13.11 Capping Peptides Directed to Pathological Aggregates of Mutant Alpha-Synuclein (A53T Mutation)

A mutant form of alpha-synuclein (A53T mutation) is a polypeptide which can form pathological aggregates. Pathological aggregates of alpha-synuclein are associated with Parkinson's disease, Parkinson's disease with dementia, dementia with Lewy bodies and multiple system atrophy (Chiti F and Dobson C M (2017) Annu. Rev. Biochem. 86: 27-68). Capping peptides were designed targeting a pathological aggregate of mutant alpha-synuclein (A53T mutation) as described in example 1. The amino acid sequence of alpha-synuclein is available as UniProt accession number (https://www.uniprot.org)

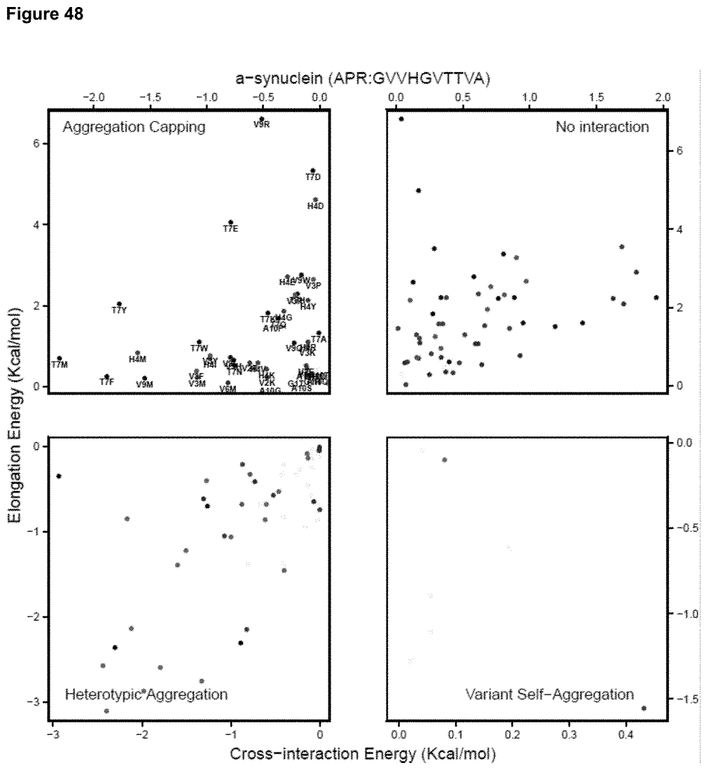

P37840. An amyloid core structure for the target APR sequence GVVHGVTTVA (SEQ ID NO: 168) present in the mutant alpha-synuclein is available in the protein structure database (www.rcsb.org) as 4znn. The analysis based on this amyloid core structure was conducted as outlined in example 1 and is shown in FIG. 48.

Three predicted capping peptides identified from the analysis on the core structure 4znn are GVVHGVTTRA (SEQ ID NO: 169), GVVHGVETVA (SEQ ID NO: 170) and GVVHMVTTVA (SEQ ID NO: 171).

Thus, non-natural molecules comprising capping peptides targeting mutant alpha-synuclein aggregates linked to a moiety targeting the intracellular proteolysis system are provided for use to treat Parkinson's disease, Parkinson's disease with dementia, dementia with Lewy bodies and multiple system atrophy.

13.12 Capping Peptides Directed to Pathological Aggregates of Immunoglobulin Light Chain Variable Domain

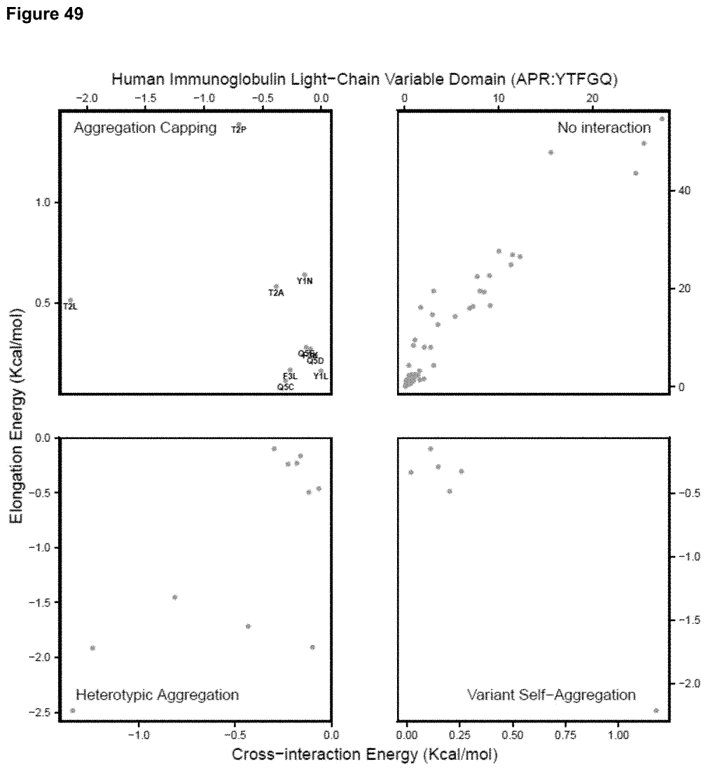

Light chain immunoglobulin variable domains is a genus (indeed different patients have different sequences of light chain variable domains) of polypeptides which can form pathological aggregates. Pathological aggregates of light chain immunoglobulin variable domains are associated with heart failure with preserved ejection fraction, nephrotic syndrome, hepatic dysfunction, peripheral/autonomic neuropathy, and atypical smoldering multiple myeloma or monoclonal gammopathy (Gertz M A (2020) Am. J. Hematol. 95(7): 848). Capping peptides were designed targeting a pathological aggregate of a light chain immunoglobulin variable domain sequence as described in example 1. The analysis based on this amyloid core structure was conducted as outlined in example 1 and is shown in FIG. 49.

Three predicted capping peptides identified from the analysis on the core structure 6diy are YPFGQ (SEQ ID NO: 173), YLFGQ (SEQ ID NO: 174) and NTFGQ (SEQ ID NO: 175).

Thus, non-natural molecules comprising capping peptides targeting a light chain immunoglobulin variable domain aggregate linked to a moiety targeting the intracellular proteolysis system are provided for use to treat heart failure with preserved ejection fraction, nephrotic syndrome, hepatic dysfunction, peripheral/autonomic neuropathy, and atypical smoldering multiple myeloma or monoclonal gammopathy.

13.13 Capping Peptides Directed to Pathological Aggregates of TAR DNA-Binding Protein 43

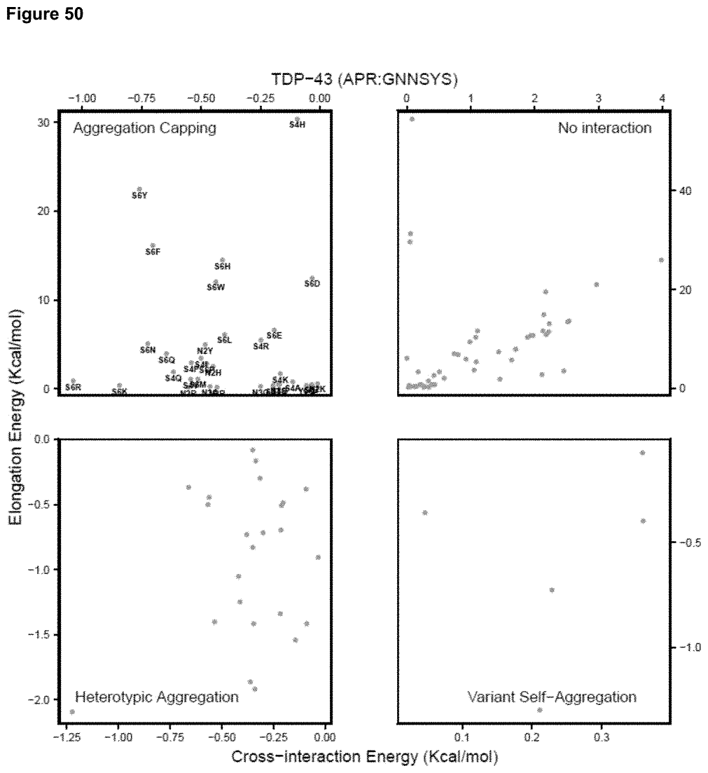

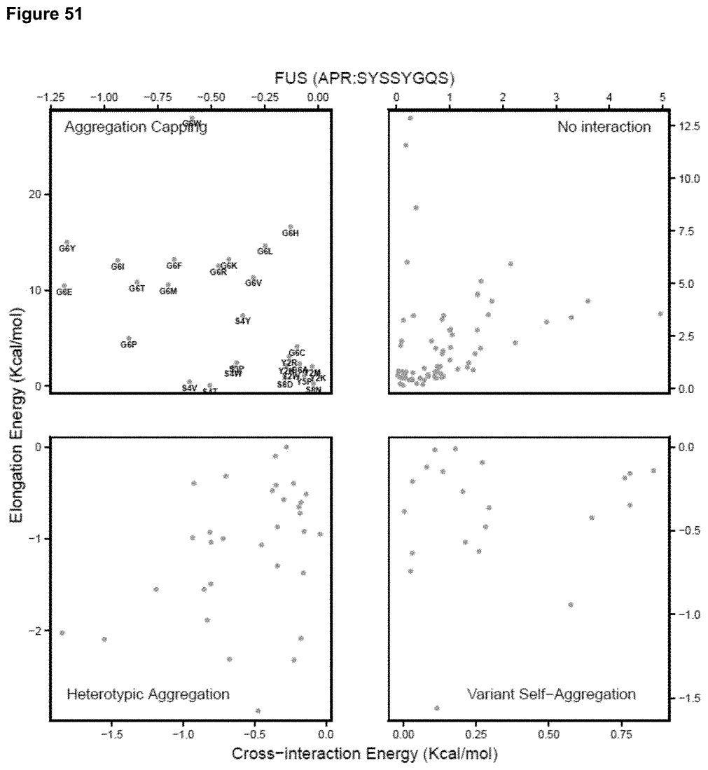

TAR DNA-binding protein 43 (TDP-43) is a protein which can form pathological aggregates. Pathological aggregates of TDP-43 are associated with frontotemporal lobar degeneration with ubiquitin-positive inclusions and amyotrophic lateral sclerosis (ALS) (Chiti F and Dobson C M (2017) Annu. Rev. Biochem. 86: 27-68). Capping peptides were designed targeting pathological aggregates of TDP-43 as described in example 1. The amino acid sequence of TDP-43 is available as UniProt accession number (https://www.uniprot.org) Q13148. An amyloid core structure for the target TDP-43 sequence GNNSYS (SEQ ID NO: 176) present in TDP-43 is available in the protein structure database (www.rcsb.org) as 5wia. The analysis based on this amyloid core structure conducted as outlined in example 1 is shown in FIG. 50.

Three predicted capping peptides identified from the analysis are GNNSYY (SEQ ID NO: 177), GNNHYS (SEQ ID NO: 178) and GNNSYF (SEQ ID NO: 179).

Thus, non-natural molecules comprising capping peptides targeting pathological aggregates of TDP-43 linked to a moiety targeting the intracellular proteolysis system are provided for use to treat frontotemporal lobar degeneration with ubiquitin-positive inclusions and amyotrophic lateral sclerosis (ALS).

13.14 Capping Peptides Directed to Pathological Aggregates of RNA-Binding Protein FUS

The RNA-binding protein FUS (FUS) is a protein which can form pathological aggregates. Pathological aggregates of FUS are associated with frontotemporal lobar degeneration with ubiquitin-negative inclusions and amyotrophic lateral sclerosis (ALS) (Chiti F and Dobson C M (2017) Annu. Rev. Biochem. 86: 27-68). Capping peptides were designed targeting a pathological aggregate of FUS as described in example 1. The amino acid sequence of FUS is available as UniProt accession number (https://www.uniprot.org) P35637. An amyloid core structure for the target APR sequence SYSSYGQS (SEQ ID NO: 180) present in FUS is available in the protein structure database (www.rcsb.org) as 6bxv. The analysis based on this amyloid core structure conducted as outlined in example 1 is shown in FIG. 51.

Three predicted capping peptides identified from the analysis are SYSSYWQS (SEQ ID NO: 181), SYSSYYQS (SEQ ID NO: 182) and SYSSYIQS (SEQ ID NO: 183).

Thus, non-natural molecules comprising capping peptides targeting pathological aggregates of FUS linked to a moiety targeting the intracellular proteolysis system are provided for use to treat frontotemporal lobar degeneration with ubiquitin-negative inclusions and amyotrophic lateral sclerosis (ALS).

TABLE 7
summary of the capping peptide identification for additional
proteins capable of forming pathological aggregates

| | PDB | UniProt | | Exemplified capping |
|---|---|---|---|---|
| Target protein | ID | ID | APR sequence | peptides |
| Transthyretin | 2m5n | P02766 | SEQ ID NO: 92 | SEQ ID NO: 93, 94 and 95 |
| Insulin | 2omp | A6XGL2 | SEQ ID NO: 96 | SEQ ID NO: 98, 99 and 100 |
| Insulin | 3hyd | A6XGL2 | SEQ ID NO: 97 | SEQ ID NO: 101, 102 and 103 |
| IAPP | 3fod | P10997 | SEQ ID NO: 104 | SEQ ID NO: 110, 111 and 112 |
| IAPP | 3ftl | P10997 | SEQ ID NO: 105 | SEQ ID NO: 113, 114 and 115 |
| IAPP | 3ftr | P10997 | SEQ ID NO: 106 | SEQ ID NO: 116, 117 and 118 |
| IAPP | 5e5v | P10997 | SEQ ID NO: 107 | SEQ ID NO: 119, 120 and 121 |
| IAPP | 5e5x | P10997 | SEQ ID NO: 108 | SEQ ID NO: 122, 123 and 124 |
| IAPP | 5e5z | P10997 | SEQ ID NO: 109 | SEQ ID NO: 125, 126 and 127 |
| Beta-2-microglobulin | 3loz | P61769 | SEQ ID NO: 128 | SEQ ID NO: 129, 130 and 131 |
| prostatic acid phosphatase | 3ppd | P15309 | SEQ ID NO: 132 | SEQ ID NO: 133, 134 and 135 |
| SOD1 | 4nin | P00441 | SEQ ID NO: 136 | SEQ ID NO: 138, 139 and 140 |
| SOD1 | 4nip | P00441 | SEQ ID NO: 137 | SEQ ID NO: 141, 142 and 143 |
| Lysozyme | 4r0p | H0YDZ2 | SEQ ID NO: 144 | SEQ ID NO: 145, 146 and 147 |
| Alpha-synuclein | 4ril | P37840 | SEQ ID NO: 148 | SEQ ID NO: 149, 150 and 151 |
| p53 | 4rp6 | P04637 | SEQ ID NO: 152 | SEQ ID NO: 153, 154 and 155 |
| Prion Protein | 4w5m | P04156 | SEQ ID NO: 156 | SEQ ID NO: 159, 160 and 161 |
| Prion Protein | 4w5p | P04156 | SEQ ID NO: 157 | SEQ ID NO: 162, 163 and 164 |
| Prion Protein | 4w71 | P04156 | SEQ ID NO: 158 | SEQ ID NO: 165, 166 and 167 |
| Alpha-synuclein | 4znn | P37840 | SEQ ID NO: 168 | SEQ ID NO: 169, 170 and 171 |
| (A53T mutant) | | | | |
| TDP-43 | 5wia | Q13148 | SEQ ID NO: 176 | SEQ ID NO: 177, 178 and 179 |
| FUS | 6bxv | P35637 | SEQ ID NO: 180 | SEQ ID NO: 181, 182 and 183 |
| Ig Light Chain | 6diy | Bruhmstein B el at | SEQ ID NO: 172 | SEQ ID NO: 173, 174 and 175 |
| Variable Domain | | (2018) J. Biol. Chem. | | |
| | | 293(1) 19659, FIG. 11 | | |

14. Effect of Non-Natural Molecules on the Seeding and Spreading of the Tau Pathology in Mice

In a next step the non-natural molecules of the invention, targeting tau aggregates, are used in an in vivo murine wild type model.

Thereto different types of Tau seeds are prepared:
Recombinant Tau aggregates (or tau seeds which is an equivalent term) (prepared by incubating recombinant purified Tau (10 μM) with heparine (5 μM) for at least two weeks)
Human brain-extracted Tau seeds (sarkosyl extraction is applied to purify insoluble tau aggregates from brain according to Ren Y and Sahara N. (2013) Front. Neurol. 4: 102)
PS19 mouse brain-extracted Tau seeds (The PS19 mouse model harbors the T34 isoform of microtubule-associated protein tau with one N-terminal insert and four microtubule binding repeats (1N4R) encoding the human P301S mutation, all driven by the mouse prion protein promoter. These mice are useful in studying neurofibrillary tangles, neurodegenerative tauopathy and Alzheimer's disease) were also extracted with the sarkosyl extraction method.

Each of these three different Tau aggregates are mixed with buffer (10 mM HEPES, pH 7.5, 100 mM NaCI) and a non-natural molecule of the invention (HET-JAV: WQIVYKGSVWMINKGSGSNLLRLTGW, SEQ ID NO: 22) at concentration ranges between 1 and 10 μM was dissolved in this buffer.

Wild type mice (C57BL/6jax) are sedated with isoflurane. Sedated mice are fixed and injected with 2 μL tau seeds (mixed with buffer alone or mixed with the non-natural molecule) at specific stereotactic coordinates to inject in the hippocampus (X=Medial/Lateral+1.6, Y=Anterior/Posterior−2.2 and Z=Dorsal/Ventral−1.5).

In a next step at different time points after injection (2 h, 12 h and 24 h), mice are sacrificed, perfused and brain samples are collected for staining and imaging.

Recombinant seeds are labeled with a fluorescent dye (atto-633) and can be readily detected and quantified without staining. To detect brain-extracted tau seeds staining with a Tau antibody (Source: Alzforum, Cat# MAB361) is required to detect and quantify the seeds.

We observe a significant reduction in the amount of Tau seeds in the hippocampus in the condition in which the Tau seeds are pretreated with a non-natural molecule of the invention as compared to the condition of Tau seeds not pretreated with a non-natural molecule.

In an alternative transgenic murine model preformed tau aggregates are pretreated with the non-natural molecule (SEQ ID NO: 22) and after an incubation of 6 hours, this preformed mixture is injected into the hippocampus of young hTau^{[P301L]} mice (see Kent BA et al (2017) Brain Behay. 8(1) e00896), which haven't developed any tau pathology yet. Nine weeks after injection mice are sacrificed. Immunohistochemical (IHC) quantification of AT8-positive inclusions in the ipsilateral (injection site) and contralateral hippocampus (opposite site) is conducted. Preformed tau aggregates pretreated with a buffer serve as a control vehicle. We observe that the level of tau pathology spread to the contralateral hippocampus is reduced when the non-natural molecule depicted in SEQ ID NO: 22 is applied as compared to the vehicle.

1. A non-natural molecule comprising at least one of structure (A), (B) or (C):
Z₀-X₁-CP1-X₂-Z₁-M1-Z₂ (A)
Z₀-X₁-CP1-X₂-Z₁-X₃-CP2-X₄-Z₂-M1-Z₃ (B)
Z₀-X₁-CP1-X₂-Z₁-X₃-CP2-X₄-Z₂-X₅-CP3-X₆-Z₃-M1-Z₄ (C)
wherein:
Z₀ is a linker or nothing,
CP1, CP2, and CP3 are identical or different capping peptides,
wherein a capping peptide is a peptide comprising a variant of an aggregation prone region sequence (APR sequence) which APR sequence has a length of 5 to 10 amino acids and is naturally present in the amino acid sequence of a target protein which can form pathological aggregates,
wherein the variant of the APR sequence has one, two or three amino acid sequence differences as compared to the natural APR sequence present in the target protein, and
wherein the capping peptide has a negative delta G free energy for cross interaction with the three-dimensional structure of amyloid fibrils formed by the APR sequence and a positive delta G free energy for elongation with the three-dimensional structure of the amyloid fibrils formed by the APR sequence,
M1 is a moiety consisting of either a small molecule or a peptide binding to a protein involved in intracellular proteolytic degradation,
in molecule (A) Z₁ is a linker and Z₂ is selected from a linker or nothing, in molecule (B) Z₁ and Z₂ are each independently a linker and Z₃ is selected from a linker or nothing, in molecule (C) Z₁, Z₂ and Z₃ are each independently a linker and Z₄ is selected from a linker or nothing.

2. The non-natural molecule of claim wherein a second moiety (M2) is fused adjacent to the M1 moiety in the structures (A), (B) or (C), and wherein M2 binds to a protein involved in intracellular degradation.

3. The non-natural molecule of claim 1, wherein in the molecules (B) and (C) the capping peptides are directed to the same or different aggregation prone regions of the pathological aggregation forming target protein.

4. The non-natural molecule of claim 1, wherein in the molecules (B) and (C) the capping peptides are directed to aggregation prone regions of different pathological aggregating forming target proteins.

5. The non-natural molecule of claim 1, wherein the pathological aggregate is an amyloid or non-amyloid aggregate.

6. The non-natural molecule of claim 1, wherein the protein involved in intracellular proteolytic degradation belongs to the ubiquitin proteasome degradation system (UPS).

7. The non-natural molecule of claim 1, wherein the protein involved in intracellular proteolytic degradation belongs to the autophagy system.

8. The non-natural molecule of claim 1, wherein the pathological aggregation forming target protein is an amyloid forming target protein and is selected from the list consisting of tau, IAPP, amyloid-beta, huntingtin-1 and alfa-synuclein.

9. The non-natural molecule of claim 1, wherein the pathological aggregation forming target protein is a non-amyloid forming target protein and is selected from the list consisting of FUS, TDP-43, ataxin-1 and p53.

10. The non-natural molecule according of claim 1, wherein the non-natural molecule is comprised in a medicament.

11. A method to obtain a set of candidate capping peptides binding to a target protein that forms pathological aggregates, the method comprising:
a. obtaining the 3-dimensional (3-D) structure of fibrils produced by an aggregation prone region (APR) amino acid sequence isolated from a target protein that can form pathological aggregates,
b. generating an in silico list of variants of said APR amino acid sequence wherein each variant has 1, or 2, or 3 amino acid differences as compared to the natural APR amino acid sequence,
c. calculating with a Forcefield algorithm the thermodynamic stability for every variant sequence for the interactions between i) the variant sequence and the 3-D structure of the fibrils produced by the APR sequence, this value is designated as the delta Gibbs energy of cross-interaction and ii) the variant sequence and a 3-D structure of fibrils produced by the APR sequence with a variant sequence interacting at its axial end, this value is designated as the delta Gibbs energy of elongation,
d. obtaining at set of candidate capping peptides wherein candidates have a negative delta G free energy for cross-interaction and a positive delta G free energy for elongation; and
e. experimentally testing the set of candidate capping peptides and producing one or more capping peptides.

12. (canceled)

13. The non-natural molecule of claim 1, wherein the variant of the APR sequence contains at least one D-amino acid and/or at least one artificial amino acid.
